(19) United States
(12) Patent Application Publication
(10) Pub. No.: US 2022/0226454 A1
(43) Pub. Date: Jul. 21, 2022

(54) ANTIGENIC COMPOSITION(S) AND METHOD(S) OF USE AGAINST PORPHYROMONAS GINGIVALIS FOR THE PREVENTION AND/OR TREATMENT OF INFECTION AND/OR DISEASES

(71) Applicants: KEYSTONE BIO, INC., St. Louis, MO (US); UNIVERSITY OF LOUISVILLE RESEARCH FOUNDATION, INC., Louisville, KY (US)

(72) Inventors: Peter L. Nara, Senecaville, OH (US); Jan Stanislaw Potempa, Louisville, KY (US); John Knox Tobin, Middletown, MD (US); Gregory John Tobin, Frederick, MD (US)

(21) Appl. No.: 17/647,571

(22) Filed: Jan. 10, 2022

Related U.S. Application Data
(60) Provisional application No. 63/135,876, filed on Jan. 11, 2021.
(60) Provisional application No. 63/221,394, filed on Jul. 13, 2021.

(51) Int. Cl. A61K 39/02; A61K 45/06; A61P 31/04

(52) U.S. Cl. CPC A61K 39/0216; A61P 31/04; A61K 45/06

(57) ABSTRACT

Vaccines for Porphyromonas gingivalis are described. Also provided are methods of treating or preventing a disorder or disease by administering the vaccine.

### CROSS REFERENCE TO RELATED APPLICATIONS

This application claims priority to U.S. Prov. App. No. 63/221,394 filed Jul. 13, 2021 entitled “ANTIGENIC COMPOSITIONS(S) AND METHOD(S) OF USE AGAINST PORPHYROMONAS GINGIVALIS FOR THE PREVENTION AND/OR TREATMENT OF INFECTION AND/OR DISEASES.”, and to U.S. Prov. App. No. 63/135,876 filed Jan. 11, 2021, entitled “ANTIGENIC COMPOSITIONS(S) AND METHOD(S) OF USE AGAINST PORPHYROMONAS GINGIVALIS FOR THE PREVENTION AND/OR TREATMENT OF INFECTION AND/OR DISEASES,” which are each incorporated by reference in their entirety.

### REFERENCE TO SEQUENCE LISTING

[The present application is being filed along with a Sequence Listing in electronic format. The Sequence Listing is provided as a file entitled SeqList_KEYBI006A created on Jan. 10, 2022, which is 319,688 bytes in size. The information in the electronic format of the Sequence Listing is incorporated herein by reference in its entirety.

### FIELD

The present disclosure generally relates to both a complex of bacterial proteins to antigenic peptides and/or proteins (APs) to raise an innate and acquired immune response against Porphyromonas gingivalis, and the treatment and/or prevention of systemic diseases associated with the chronic inflammation and/or periodontal disease(s) associated with P. gingivalis infection and/or the continuous release of the free soluble and/or outer-membrane associated vesicles (OMV) containing various complex exo-toxins, using such P. gingivalis bacterial antigens and/or the OMV containing exotoxin antigen-binding molecules, e.g., biomolecules.

### DESCRIPTION OF THE RELATED ART

Porphyromonas gingivalis is a gram-negative anaerobic, asaccharolytic, red complex bacteria. P. gingivalis can infect and remain permanently in the oral cavity as a polymicrobial biofilm and/or translocate to other body cells/tissues. Upon infection, P. gingivalis can produce and secrete/excrete outer membrane vesicles (containing a complex of gingipains, hemagglutinin, adhesins and LPS) and in free soluble form into the gingival sulcus space with its attending fluid, blood and lymphatic circulation. P. gingivalis infection can lead to a subacute to chronic state of oral and systemic dysbiosis (pathological and abnormal change from the normal oral flora/microbiota) and subsequent chronic local and systemic infection/disease(s), further leading to increased local and distant vascular and tissue and organ inflammation throughout the entire body. Certain end organs, e.g., heart vessels, carotid arteries, vessels in the brain, the brain itself and supporting structures, liver, pancreas, gastro-intestinal tract, joints, lungs, pancreas, reproductive system, etc., are more affected than others. P. gingivalis-induced inflammation is implicated in diseases such as cardiovascular disease, heart attack, atherosclerosis, stroke, various early and later cognitive dementias, early and later neuro-cognitive decline, Alzheimer's disease, diabetes, NASH, rheumatoid arthritis, insulin resistance, pre-term birth, etc.

### SUMMARY

Disclosed herein is an antigenic composition capable of raising an immune response against P. gingivalis in a subject. In some embodiments, the composition comprises an at least one isolated and purified peptide and/or protein, wherein the isolated and purified peptide and/or protein comprises an epitope that is bound by the antigen binding molecule KB001. In some embodiments, the peptide and/or protein comprises a sequence having at least 80% identity to the sequence YTYTVYRDGTKIK (SEQ ID NO: 6). In some embodiments, the peptide and/or protein is not complexed with other Arg-specific and Lys-specific P. gingivalis proteinase and adhesin proteins.

Also disclosed herein is a composition for use in raising an immune response directed against P. gingivalis in a subject. In some embodiments, the composition comprises an effective amount of an at least one isolated and purified peptide and/or protein, wherein the isolated and purified peptide and/or protein comprises an epitope that is bound by the antigen binding molecule KB001.

Also disclosed herein is a composition for use in the prevention of a disease or disorder arising from the infection of P. gingivalis in a subject. In some embodiments, the composition comprises an effective amount of an at least one isolated and purified peptide and/or protein, wherein the isolated and purified peptide and/or protein comprises an epitope that is bound by the antigen binding molecule KB001.

Also disclosed herein is a composition for use as a therapeutic against a disease or disorder arising from the infection of P. gingivalis in a subject. In some embodiments, the composition comprises an effective amount of an at least one isolated and purified peptide and/or protein, wherein the isolated and purified peptide and/or protein comprises an epitope that is bound by the antigen binding molecule KB001.

In some embodiments, the peptide and/or protein comprises a sequence having at least 80% identity to the sequence YTYTVYRDGTKIK (SEQ ID NO: 6). In some embodiments, the composition further comprises a pharmaceutically acceptable carrier. In some embodiments, the composition further comprises an adjuvant. In some embodiments, the adjuvant is an aluminum salt adjuvant. In some embodiments, the composition is formulated for use orally. In some embodiments, the composition is formulated for percutaneous administration. In some embodiments, the subject is mammalian and/or human. In some embodiments, the peptide and/or protein comprises one, two, three, and/or four sequences having at least 80% identity to one, two, three, and/or all four of the sequences:

(a)
(SEQ ID NO: 1)
GVSPKVCKDVTVEGSNEFAPVQNLT,
(b)
(SEQ ID NO: 2)
YCVEVKYTAGVSPK,
(c)
(SEQ ID NO: 3)
AGTYDFAIAAPQANAKIWIAGQGPTKEDDYVFEAGKKYHFLMKKMGSGDG
TELTISEGGGSDYTYTVYRDGTKIKEGLTATTFEEDGVAAGNHEYCVEVK
YTAGVSPKVCKDVTVEGSNEFAPVQNLT,
and/or
(d)
(SEQ ID NO: 4)
GVSPK.

In some embodiments, the epitope is a conformational epitope defined by the sequence YTYTVYRDGTKIK (SEQ ID NO: 6). In some embodiments, the epitope is a conformational epitope defined by the sequence YTYTVYRDGTKIK (SEQ ID NO: 6), wherein a structure of the confirmational epitope is effectively the same as a structure of YTYTVYRDGTKIK (SEQ ID NO: 6) within a sequence of Kgp. In some embodiments, the epitope is a conformational epitope defined by the sequence AGTYDFAIAAPQANAKIWIAGQGPTKEDDYVFEAGKKYHFLMKKMGS GDGTELTIS EGGGSDYTYTVYRDGTKIKEGLTATTFEEDGVAAGNHEYCVEVKYTAGVSPKVCK DVTVEGSNEFAPVQNLT (SEQ ID NO: 3), wherein a structure of the confirmational epitope is effectively the same as a structure of AGTYDFAIAAPQANAKIWIAGQGPTKEDDYVFEAGKKYHFLMKKMGS GDGTELTIS EGGGSDYTYTVYRDGTKIKEGLTATTFEEDGVAAGNHEYCVEVKYTAGVSPKVCK DVTVEGSNEFAPVQNLT (SEQ ID NO: 3), within a sequence of Kgp. In some embodiments, the peptide and/or protein is of 50 or fewer amino acids. In some embodiments, the peptide is no longer than 20 amino acids in length. In some embodiments, the peptide is no longer than 14 amino acids in length.

Also disclosed herein is a method of treating a disease or condition associated with the presence of P. gingivalis in an oral tissue of a subject. In some embodiments, the method comprises administering to a subject a composition of claim 1 and administering to the subject a peptide wherein the polypeptide comprises: YTYTVYRDGTKIK (SEQ ID NO: 6), a polypeptide having at least 80% identity to this sequence, an immunogenic fragment of 8, 9, 10, 12, 13, or more consecutive amino acids of this sequence, and a variant comprising a conservative substitution of at least one amino acid of this sequence. In some embodiments, the immunogenic composition is administered at least two times.

Also disclosed herein is a method of treating or preventing a vascular disease or symptoms thereof. In some embodiments, the method comprises identifying a subject in need of treating or preventing a vascular disease or symptoms thereof, and administering to the subject a therapeutically effective amount of the immunogenic composition disclosed herein, thereby treating or preventing the vascular disease or symptoms thereof. In some embodiments, the vascular disease comprises cardiovascular disease, atherosclerosis, coronary artery disease, myocardial infarction, stroke, and myocardial hypertrophy. In some embodiments, the method further comprises administering to the subject at least one other therapeutic agent for treating or preventing the vascular disease, or symptoms thereof. In some embodiments, the other therapeutic agent comprises a serum lipid lowering agent. In some embodiments, the other therapeutic agent is a statin.

Also disclosed herein is a method of treating or preventing a vascular disease or symptoms thereof. In some embodiments, the method comprises administering to a subject in need of treating or preventing a vascular disease, or symptoms thereof, a therapeutically effective amount of at least one therapeutic agent for treating or preventing the vascular disease, or symptoms thereof, and administering an effective amount of the immunogenic composition disclosed herein, to thereby enhance the therapeutic effect of the at least one therapeutic agent. In some embodiments, the other therapeutic agent comprises a serum lipid lowering agent. In some embodiments, the other therapeutic agent is a statin.

Also disclosed herein is method of treating or preventing a systemic disease or symptoms thereof. In some embodiments, the method comprises identifying a subject in need of treating or preventing a systemic disease or symptoms thereof, wherein the systemic disease is one or more of type II diabetes, insulin resistance and metabolic syndrome, and administering to the subject a therapeutically effective amount of the immunogenic composition disclosed herein, thereby treating or preventing the systemic disease or symptoms thereof.

Also disclosed herein is a method of treating or preventing rheumatoid arthritis or symptoms thereof. In some embodiments, the method comprises identifying a subject in need of treating rheumatoid arthritis or symptoms thereof, and administering to the subject a therapeutically effective amount of the immunogenic composition disclosed herein, thereby treating or preventing the rheumatoid arthritis or symptoms thereof.

Also disclosed herein is a method of treating or preventing cancer or symptoms thereof. In some embodiments, the method comprises identifying a subject in need of treating cancer or symptoms thereof, and administering to the subject a therapeutically effective amount of the immunogenic composition disclosed herein, thereby treating or preventing the cancer or symptoms thereof. In some embodiments, the cancer is oral, gastrointestinal, lung or pancreatic cancer. In some embodiments, the method further comprises administering to the subject at least one other therapeutic agent for treating or preventing the cancer, or symptoms thereof. In some embodiments, the other therapeutic agent comprises a small molecule drug or immunotherapeutic agent.

Also disclosed herein is a method of treating or preventing cancer or symptoms thereof. In some embodiments, the method comprises administering to a subject in need of treating or preventing cancer, or symptoms thereof, a therapeutically effective amount of at least one therapeutic agent for treating or preventing the cancer, or symptoms thereof, and administering an effective amount of the immunogenic composition disclosed herein, to thereby enhance the therapeutic effect of the at least one therapeutic agent. In some embodiments, the at least one therapeutic agent comprises a small molecule drug or immunotherapeutic agent. In some embodiments, the cancer is oral, gastrointestinal, lung or pancreatic cancer.

Also disclosed herein is a method of treating or preventing a gut microbiome-related disorder or symptoms thereof. In some embodiments, the method comprises identifying a subject in need of treating a gut microbiome-related disorder or symptoms thereof, and administering to the subject a therapeutically effective amount of the immunogenic composition disclosed herein, thereby treating or preventing the gut microbiome-related disorder or symptoms thereof. In some embodiments, the gut microbiome-related disorder comprises inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity.

Also disclosed herein is a method of treating or preventing a cognitive disorder or symptoms thereof. In some embodiments, the method comprises identifying a subject in need of treating a cognitive disorder or symptoms thereof, and administering to the subject a therapeutically effective amount of the immunogenic composition disclosed herein, thereby treating or preventing the cognitive disorder or symptoms thereof. In some embodiments, the cognitive disorder is Alzheimer's disease. In some embodiments, the cognitive disorder is early, middle or late dementia.

Also disclosed herein is a method of treating or preventing an age-related or longevity-related disorder, or symptoms thereof. In some embodiments, the method comprises identifying a subject in need of treating an age-related or longevity-related disorder, and administering to the subject a therapeutically effective amount of the immunogenic composition disclosed herein, thereby treating or preventing the age-related or longevity-related disorder, or symptoms thereof.

Also disclosed herein is a method of treating or preventing a post event myocardial hypertrophy or symptoms thereof. In some embodiments, the method comprises identifying a subject in need of treating or preventing a post event myocardial hypertrophy or symptoms thereof, and administering to the subject a therapeutically effective amount of the immunogenic composition disclosed herein, thereby treating or preventing the post event myocardial hypertrophy or symptoms thereof.

Also disclosed herein is method of treating a wound. In some embodiments, the method comprises identifying a subject in need of treating a wound; and administering to the subject a therapeutically effective amount of the immunogenic composition disclosed herein, whereby closure of the wound is enhanced, thereby treating the wound.

Also disclosed herein is a method of treating or preventing an age-related macular degeneration (AMD) or symptoms thereof. In some embodiments, the method comprises identifying a subject in need of treating or preventing AMD or symptoms thereof, and administering to the subject a therapeutically effective amount of the immunogenic composition disclosed herein, thereby treating or preventing the AMD or symptoms thereof.

Also disclosed herein is a method of treating or preventing an aneurysm or symptoms thereof. In some embodiments, the method comprises identifying a subject in need of treating or preventing an aneurysm or symptoms thereof, and administering to the subject a therapeutically effective amount of the immunogenic composition disclosed herein, thereby treating or preventing the aneurysm or symptoms thereof. In some embodiments, the aneurysm is a cerebral or abdominal aneurysm.

Also disclosed herein is a method of treating or preventing a glioma or symptoms thereof. In some embodiments, the method comprises identifying a subject in need of treating or preventing a glioma or symptoms thereof, and administering to the subject a therapeutically effective amount of the immunogenic composition disclosed herein, thereby treating or preventing the glioma or symptoms thereof.

Also disclosed herein is a method of treating or preventing a large vessel stroke C-IMT or symptoms thereof. In some embodiments, the method comprises identifying a subject in need of treating or preventing a large vessel stroke C-IMT or symptoms thereof, and

administering to the subject a therapeutically effective amount of the immunogenic composition disclosed herein, thereby treating or preventing the large vessel stroke C-IMT or symptoms thereof.

Also disclosed herein is a method of treating or preventing microvascular defects and associated dementias, or symptoms thereof. In some embodiments, the method comprises identifying a subject in need of treating or preventing microvascular defects and associated dementias, or symptoms thereof, and administering to the subject a therapeutically effective amount of the immunogenic composition disclosed herein, thereby treating or preventing the microvascular defects and associated dementias, or symptoms thereof. In some embodiments, the microvascular defects and associated dementias comprises microvascular defects Parkinson's.

Also disclosed herein is a method of treating or preventing a peri-implantitis or symptoms thereof. In some embodiments, the method comprises identifying a subject in need of treating or preventing a peri-implantitis or symptoms thereof, and administering to the subject a therapeutically effective amount of the immunogenic composition disclosed herein, thereby treating or preventing the peri-implantitis or symptoms thereof.

Also disclosed herein is method of treating or preventing a renal disease or symptoms thereof. In some embodiments, the method comprises identifying a subject in need of treating or preventing a renal disease or symptoms thereof, and administering to the subject a therapeutically effective amount of the immunogenic composition disclosed herein, thereby treating or preventing the renal disease or symptoms thereof.

Also disclosed herein is a method of treating or preventing a regenerative and stem cell dysfunction, or symptoms thereof. In some embodiments, the method comprises identifying a subject in need of treating or preventing a regenerative and stem cell dysfunction, or symptoms thereof, and administering to the subject a therapeutically effective amount of the immunogenic composition disclosed herein, thereby treating or preventing the regenerative and stem cell dysfunction, or symptoms thereof.

Also disclosed herein is a method of treating or preventing a condition, disorder or disease associated with a P. gingivalis infection, or symptoms thereof. In some embodiments, the method comprises identifying a subject in need of treating or preventing a condition, disorder or disease associated with a P. gingivalis infection, or symptoms thereof, and administering to the subject a therapeutically effective amount of the immunogenic composition disclosed herein, thereby treating or preventing the condition, disorder or disease associated with a P. gingivalis infection, or symptoms thereof. In some embodiments, the method further comprises administering the therapeutically effective amount of the immunogenic composition to treat the condition, disorder or disease associated with a P. gingivalis infection, or symptoms thereof. In some embodiments, the method further comprises administering the therapeutically effective amount of the immunogenic composition to prevent the condition, disorder or disease associated with a P. gingivalis infection, or symptoms thereof. In some embodiments, the condition, disorder or disease is associated with a local infection of P. gingivalis. In some embodiments, the condition, disorder or disease is associated with an oral infection of P. gingivalis. In some embodiments, the condition, disorder or disease is one or more of: vascular disease (e.g., cardiovascular disease, atherosclerosis, coronary artery disease, myocardial infarction, stroke, and myocardial hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD (age-related macular degeneration), cerebral and abdominal aneurysms, glioma, large vessel stroke C-IMT, microvascular defects and associated dementias (e.g., Parkinson's), Peri-Implantitis and/or periodontal disease and/or associated bone loss, cognitive disorders (e.g., early, middle, and/or late dementia; Alzheimer's disease); regenerative and stem cell dysfunction; and longevity or age-related disorder. In some embodiments, the administering comprises administering the immunogenic composition intravenously, subgingivally, intradermally, subcutaneously, intrathecally, or by nebulization.

Also disclosed herein is a use of an immunogenic composition disclosed herein, for treatment of a disorder associated with, caused by or complicated by P. gingivalis. In some embodiments, the disorder associated with, caused by or complicated by P. gingivalis is one or more of: vascular disease (e.g., cardiovascular disease, atherosclerosis, coronary artery disease, myocardial infarction, stroke, and myocardial hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD (age-related macular degeneration), cerebral and abdominal aneurysms, glioma, large vessel stroke C-IMT, microvascular defects and associated dementias (e.g., Parkinson's), Peri-Implantitis and/or periodontal disease and/or associated bone loss, cognitive disorders (e.g., early, middle, and/or late dementia; Alzheimer's disease); regenerative and stem cell dysfunction; and longevity or age-related disorder.

Also disclosed herein is a method of screening for a vaccine. In some embodiments, the method comprises providing an antibody that binds to a Porphyromonas gingivalis protein PG0495, determining an epitope to which the antibody binds, and providing a fragment of the Porphyromonas gingivalis protein PG0495 as a protein fragment. In some embodiments, the epitope is a linear epitope. In some embodiments, the epitope is a conformational epitope. In some embodiments, the fragment is provided in a great enough concentration to be therapeutically effective.

Also disclosed herein is a prokaryotic or eucaryotic cell, which comprises a recombinant polynucleotide consisting of nucleotides encoding the polypeptide of at least one ofa polypeptide of YTYTVYRDGTKIK (SEQ ID NO: 6), a polypeptide having at least 80% identity to SEQ ID NO: 6, an immunogenic fragment of 8, 9, 10, 12, 13, or more consecutive amino acids of SEQ ID NO: 6, and a variant comprising a conservative substitution of at least one amino acid of SEQ ID NO: 6, wherein the polynucleotide is operatively linked to at least one regulatory element.

In some embodiments, the composition of any embodiment disclosed herein comprises at least one amino acid from HagA and/or gingipiain. In some embodiments, the protein, epitope, polypeptide, amino acid sequence, or immunogenic fragment comprises at least one additional amino acid from HagA and/or gingipiain. In some embodiments, the AP contains a set of one or more paired cysteines. In some embodiments, the sequence of the peptide is at least 40, 50, 60, 70, 80, 90, 95, 96, 97, 98, 99, or 100% identical to the sequence in FIG. 4 or 5A, 5B, or 5C. In some embodiments, the sequence of the peptide comprises at least YTYTVYRDGTKIK (SEQ ID NO: 6).

In some aspects, provided herein is an antigenic composition comprising an at least one isolated and purified peptide and/or protein, wherein the isolated and purified peptide and/or protein comprises an epitope that is bound by the antigen binding molecule KB001, wherein the isolated and purified peptide and/or protein is capable of raising an immune response against P. gingivalis in a subject.

In some aspects, provided herein is a composition for use in raising an immune response directed against P. gingivalis in a subject, the composition comprising an effective amount of an isolated and purified peptide and/or protein, wherein the isolated and purified peptide and/or protein comprises an epitope that is bound by the antigen binding molecule KB001, and wherein optionally, the epitope includes at least one of the residues in YTYTVYRDGTKIK (SEQ ID NO: 6).

In some aspects, provided herein is a composition for use in the prevention of a disease or disorder arising from the infection of P. gingivalis in a subject, the composition comprising an effective amount of an at least one isolated and purified peptide and/or protein, wherein the isolated and purified peptide and/or protein comprises an epitope that is bound by the antigen binding molecule KB001, and wherein optionally, the epitope includes at least one of the residues in YTYTVYRDGTKIK (SEQ ID NO: 6).

In some aspects, provided herein is a composition for use as a therapeutic against a disease or disorder arising from the infection of P. gingivalis in a subject, the composition comprising an effective amount of an at least one isolated and purified peptide and/or protein, wherein the isolated and purified peptide and/or protein comprises an epitope that is bound by the antigen binding molecule KB001, and wherein optionally, the epitope includes at least one of the residues in YTYTVYRDGTKIK (SEQ ID NO: 6).

In some aspects, provided herein is a method of treating a disease or condition associated with the presence of P. gingivalis in an oral tissue of a subject, comprising administering to a subject a composition of any one of the compositions herein and administering to the subject a peptide wherein the polypeptide comprises: YCVEVKYTAGVSPK or YTYTVYRDGTKIK (SEQ ID NO: 6), a polypeptide having at least 80% identity to this sequence, an immunogenic fragment of 8, 9, 10, 12, 13, or more consecutive amino acids of this sequence, and a variant comprising a conservative substitution of at least one amino acid of this sequence.

In some aspects, provided herein is a method of treating or preventing a vascular disease or symptoms thereof, comprising: identifying a subject in need of treating or preventing a vascular disease or symptoms thereof; and administering to the subject a therapeutically effective amount of the immunogenic composition provided herein, thereby treating or preventing the vascular disease or symptoms thereof.

In some aspects, provided herein is a method of treating or preventing a vascular disease or symptoms thereof, comprising: administering to a subject in need of treating or preventing a vascular disease, or symptoms thereof, a therapeutically effective amount of at least one therapeutic agent for treating or preventing the vascular disease, or symptoms thereof; and administering an effective amount of the immunogenic composition provided herein, to thereby enhance the therapeutic effect of the at least one therapeutic agent.

In some aspects, provided herein is a method of treating or preventing a systemic disease or symptoms thereof, comprising: identifying a subject in need of treating or preventing a systemic disease or symptoms thereof, wherein the systemic disease is one or more of type II diabetes, insulin resistance and metabolic syndrome; and administering to the subject a therapeutically effective amount of the immunogenic composition provided herein, thereby treating or preventing the systemic disease or symptoms thereof.

In some aspects, provided herein is a method of treating or preventing rheumatoid arthritis or symptoms thereof, comprising: identifying a subject in need of treating rheumatoid arthritis or symptoms thereof; and administering to the subject a therapeutically effective amount of the immunogenic composition provided herein, thereby treating or preventing the rheumatoid arthritis or symptoms thereof.

In some aspects, provided herein is a method of treating or preventing cancer or symptoms thereof, comprising: identifying a subject in need of treating cancer or symptoms thereof; and administering to the subject a therapeutically effective amount of the immunogenic composition provided herein, thereby treating or preventing the cancer or symptoms thereof.

In some aspects, provided herein is a method of treating or preventing cancer or symptoms thereof, comprising: administering to a subject in need of treating or preventing cancer, or symptoms thereof, a therapeutically effective amount of at least one therapeutic agent for treating or preventing the cancer, or symptoms thereof; and administering an effective amount of the immunogenic composition provided herein, to thereby enhance the therapeutic effect of the at least one therapeutic agent.

In some aspects, provided herein is a method of treating or preventing a gut microbiome-related disorder or symptoms thereof, comprising: identifying a subject in need of treating a gut microbiome-related disorder or symptoms thereof; and administering to the subject a therapeutically effective amount of the immunogenic composition provided herein, thereby treating or preventing the gut microbiome-related disorder or symptoms thereof.

In some aspects, provided herein is a method of treating or preventing a cognitive disorder or symptoms thereof, comprising: identifying a subject in need of treating a cognitive disorder or symptoms thereof; and administering to the subject a therapeutically effective amount of the immunogenic composition provided herein, thereby treating or preventing the cognitive disorder or symptoms thereof.

In some aspects, provided herein is a method of treating or preventing an age-related or longevity-related disorder, or symptoms thereof, comprising: identifying a subject in need of treating an age-related or longevity-related disorder; and administering to the subject a therapeutically effective amount of the immunogenic composition provided herein, thereby treating or preventing the age-related or longevity-related disorder, or symptoms thereof.

In some aspects, provided herein is a method of treating or preventing a post event myocardial hypertrophy or symptoms thereof, comprising: identifying a subject in need of treating or preventing a post event myocardial hypertrophy or symptoms thereof; and administering to the subject a therapeutically effective amount of the immunogenic composition provided herein, thereby treating or preventing the post event myocardial hypertrophy or symptoms thereof.

In some aspects, provided herein is a method of treating a wound, comprising: identifying a subject in need of treating a wound; and administering to the subject a therapeutically effective amount of the immunogenic composition provided herein, whereby closure of the wound is enhanced, thereby treating the wound.

In some aspects, provided herein is a method of treating or preventing an age-related macular degeneration (AMD) or symptoms thereof, comprising: identifying a subject in need of treating or preventing AMD or symptoms thereof; and administering to the subject a therapeutically effective amount of the immunogenic composition provided herein, thereby treating or preventing the AMD or symptoms thereof.

In some aspects, provided herein is a method of treating or preventing an aneurysm or symptoms thereof, comprising: identifying a subject in need of treating or preventing an aneurysm or symptoms thereof; and administering to the subject a therapeutically effective amount of the immunogenic composition provided herein, thereby treating or preventing the aneurysm or symptoms thereof.

In some aspects, provided herein is a method of treating or preventing a glioma or symptoms thereof, comprising: identifying a subject in need of treating or preventing a glioma or symptoms thereof; and administering to the subject a therapeutically effective amount of the immunogenic composition provided herein, thereby treating or preventing the glioma or symptoms thereof.

In some aspects, provided herein is a method of treating or preventing a large vessel stroke C-IMT or symptoms thereof, comprising: identifying a subject in need of treating or preventing a large vessel stroke C-IMT or symptoms thereof; and administering to the subject a therapeutically effective amount of the immunogenic composition provided herein, thereby treating or preventing the large vessel stroke C-IMT or symptoms thereof.

In some aspects, provided herein is a method of treating or preventing microvascular defects and associated dementias, or symptoms thereof, comprising: identifying a subject in need of treating or preventing microvascular defects and associated dementias, or symptoms thereof; and administering to the subject a therapeutically effective amount of the immunogenic composition provided herein, thereby treating or preventing the microvascular defects and associated dementias, or symptoms thereof.

In some aspects, provided herein is a method of treating or preventing a peri-implantitis or symptoms thereof, comprising: identifying a subject in need of treating or preventing a peri-implantitis or symptoms thereof; and administering to the subject a therapeutically effective amount of the immunogenic composition provided herein, thereby treating or preventing the peri-implantitis or symptoms thereof.

In some aspects, provided herein is a method of treating or preventing a renal disease or symptoms thereof, comprising: identifying a subject in need of treating or preventing a renal disease or symptoms thereof; and administering to the subject a therapeutically effective amount of the immunogenic composition provided herein, thereby treating or preventing the renal disease or symptoms thereof.

In some aspects, provided herein is a method of treating or preventing a regenerative and stem cell dysfunction, or symptoms thereof, comprising: identifying a subject in need of treating or preventing a regenerative and stem cell dysfunction, or symptoms thereof; and administering to the subject a therapeutically effective amount of the immunogenic composition provided herein, thereby treating or preventing the regenerative and stem cell dysfunction, or symptoms thereof.

In some aspects, provided herein is a method of treating or preventing a condition, disorder or disease associated with a P. gingivalis infection, or symptoms thereof, comprising: identifying a subject in need of treating or preventing a condition, disorder or disease associated with a P. gingivalis infection, or symptoms thereof; and administering to the subject a therapeutically effective amount of the immunogenic composition provided herein, thereby treating or preventing the condition, disorder or disease associated with a P. gingivalis infection, or symptoms thereof.

In some aspects, provided herein is an use of an immunogenic composition provided herein, for treatment of a disorder associated with, caused by or complicated by P. gingivalis.

In some aspects, provided herein is a method of screening for a vaccine, the method comprising: providing an antibody that binds to a Porphyromonas gingivalis protein PG0495, determining an epitope to which the antibody binds; and providing a fragment of the Porphyromonas gingivalis protein PG0495 as a protein fragment.

In some aspects, provided herein is a prokaryotic or eucaryotic cell, which comprises a recombinant polynucleotide consisting of nucleotides encoding the polypeptide of at least one of: a polypeptide of YCVEVKYTAGVSPK or YTYTVYRDGTKIK (SEQ ID NO: 6),a polypeptide having at least 80% identity to SEQ ID NO: 6, an immunogenic fragment of 8, 9, 10, 12, 13, or more consecutive amino acids of SEQ ID NO: 6, and a variant comprising a conservative substitution of at least one amino acid of SEQ ID NO: 6, wherein the polynucleotide is operatively linked to at least one regulatory element.

In some aspects, provided herein is an isolated peptide or polypeptide comprising: at least four or more amino acids from YTYTVYRDGTKIK (SEQ ID NO: 6).

In some aspects, provided herein is an amino acid sequence comprising: a) at least 1, 2, 3, 4, 5, 6, 7, 8, 9, 10, 11, 12, or all of the amino acids at any one or more of positions 1, 2, 3, 4, 5, 6, 7, 8, 9, 10, 11, 12, and/or 13 of YTYTVYRDGTKIK (SEQ ID NO: 6) (where the first Y is position 1 and the last K is position 13); and b) at least one of: i) the amino acid sequence of SEQ ID Nos. 85-87, or a variant thereof that is at least 50, 60, 70, 75, 80, 85, 90, 95, 96, 97, 98, 99, or more percent identical or similar to SEQ ID Nos. 85-87 fused together, ii) the amino acid sequence of SEQ ID No: 90, or a variant thereof that is at least 50, 60, 70, 75, 80, 85, 90, 95, 96, 97, 98, 99, or more percent identical or similar to SEQ ID Nos. 90, iii) the amino acid sequence of SEQ ID Nos. 88-89), or a variant thereof that is at least 50, 60, 70, 75, 80, 85, 90, 95, 96, 97, 98, 99, or more percent identical or similar to SEQ ID Nos. 88-89 fused together, or iv) the amino acid sequence of SEQ ID No: 84, or a variant thereof that is at least 50, 60, 70, 75, 80, 85, 90, 95, 96, 97, 98, 99, or more percent identical or similar to SEQ ID Nos. 84,wherein a) can be fused to b) or wherein a) can be a subsequence within b).

### BRIEF DESCRIPTION OF THE DRAWINGS

FIG. 1 is an image showing mapping of KB-001 mouse monoclonal antibody target binding by N-term sequencing and mass spectrometry, which can be equated to the relevant AP sections, as disclosed herein.

FIGS. 2A, 2B, 2C, 2D, 2E, 2F, 2G, 2H, 2I, and 2J are mapped protein sequences from the P. gingivalis the repeat epitope in hemagglutinin/adhesion and HagA gingipains domain (RE-HagA) protein complex specific to binding of KB-001 and the preliminary linear amino acid sequence of the KB-001 antibody binding epitope, according to some embodiments of the present disclosure, which can be equated to the AP as provided herein.

FIG. 3A shows an amino acid sequence of repeat epitope hemagglutinin/adhesion protein RE-HagA from Porphyromonas gingivalis strain ATCC 33277. Proteolytic processing sites are marked with bold font.

FIG. 3B shows amino acid sequences of the repeated domains of HagA, RgpA, and Kgp, with sequences encompassing the putative epitope of KB-001 underlined, according to some embodiments of the present disclosure. This epitope can be used to identify a common core sequence to the AP as provided herein. The HbR domain (Hemoglobin receptor binding domain) is boxed in a rectangle. Proteolytic processing sites are marked with bold font. For “Kgp_W83”, HA1 is in italic, and proteolytic processing of C-terminal HA part of Kgp W83 is not well defined. For “RgpA_W83”, sequence in italics before the boxed sequence shows HAL sequence in italics at C-terminus shows HA4, and sequence between the boxed sequence and HA4 shows HA3.

FIG. 3C shows a multiple sequence alignment of HA domains of HagA from Porphyromonas gingivalis strains W83 and ATCC 33277. Putative epitope of KB-001, according to some embodiments, is underlined. This can be equated to the AP as provided herein, in some embodiments.

FIG. 3D shows a multiple sequence alignment of RgpA, Kgp and HagA sequences.

FIG. 3E shows a multiple sequence alignment of RgpA, Kgp and HagA sequences.

FIG. 3F shows a multiple sequence alignment of putative sequence motifs in HagA (from W83 and ATCC 33277 strains) and RgpA and Kgp (from W83) encompassing the epitope recognized by KB-001, according to some embodiments of the present disclosure. This can be equated to the AP as provided herein, in some embodiments.

FIG. 4 displays amino acid and DNA sequences of recombinant GST-gingipain rGP-1 fusion protein construct. The linker between the fusion partners and a TEV protease site is shown bold and underlined. Immediately after this sequence starts the gingipain protein fragment which contains a single KB001 epitope.

FIG. 5A is a sequence of rGP-2

FIG. 5B is a comparison between rgp-1 and rGP-2.

FIG. 5C is a hydrophobicity plot of rGP-2.

FIG. 6A shows the gingipain antibody signal intensity from frontal lobe immunostaining of subjects AMC3,3, AD3,3, and AD4,4. As disclosed herein, 3,3 and 4,4 refers to the Apo E genetic status of the patient.

FIG. 6B shows the gingipain antibody signal intensity from occipital lobe immunostaining of subjects AMC3,3, AD3,3, and AD4,4.

FIG. 6C shows the gingipain antibody signal intensity from cerebellum immunostaining of subjects AMC3,3, AD3,3, and AD4,4.

FIG. 6D shows the gingipain antibody signal intensity from hippocampus immunostaining of subjects AMC3,3, AD3,3, and AD4,4.

FIG. 7A shows immunohistochemistry staining (IHC) of hippocampal tissue slices from the brain of a deceased Alzheimer's disease patient. The brain tissue is labeled for gingipain using binding by KB-001.

FIG. 7B shows imaging of AD brain tissue. The hippocampal brain tissue is labeled for gingipain using binding by KB-001.

FIG. 7C shows immunohistochemistry staining of P. gingivalis using KB001 binding to intra-cellular accumulated gingipains located in a hippocampal tissue from the brain of a deceased Alzheimer's disease patient.

FIG. 7D shows a P. gingivalis positive control human gum tissue used in brain IHC analysis.

FIG. 7E shows imaging of the frontal lobe of an AD patient. The frontal lobe is labeled for gingipain using binding by KB-001.

FIG. 7F shows a human choroid plexus IHC stained section of AD brains using KB001 (20X-left panel and 40 X-right panel).

FIG. 8 shows a phylogram of P. gingivalis strains, grouped by the presence or absence of accessory genes. The arrows mark the ten strains selected to represent the diversity of P.g. strains.

FIG. 9 shows a table of binding affinities of KB-001 at increasing concentrations against whole bacterial cells of P. gingivalis, as measured using plasmon resonance spectroscopy.

FIG. 10 shows SEM imaging of KB-001 binding to the P. gingivalis. strain W83. The left panel shows the cell surface at 500 nm magnification, using gold labeling. The middle panel shows KB-001 localization at 500 nm magnification. The right panel shows KB-001 localization at 2μm magnification.

FIG. 11 shows four alternative views of KB-001 (dark stain) binding to the inner and outer surface of OMV blebs produced by P. gingivalis. strain W83.

FIG. 12A shows the lack of binding of KB-001 to a RgpA-/KgP-gingipain knockout strain.

FIG. 12B shows the lack of binding of KB-001 to a RgpB-/KgP-gingipain knockout strain.

FIG. 13 shows a Western Blot for KB-001 binding to purified OMVs. PPAD is peptidylarginine deiminase secreted by P. gingivalis, and the deletion of this gene is known to reduce the overall production and release of OMVs, thus inhibiting the surface translocation of P. gingivalis.

FIG. 14 shows a Western Blot for KB-001 binding to Kgp/RgpA:HagA and RgpB:HagA complexes.

FIG. 15 shows a Western Blot for KB-001 in SDS-PAGE of lysates and purified proteins. WC—washed whole cell lysate; dil—sample diluted 10 times; KRAB—gingipain null strain; HagA—purified RagAB (main bands) containing HagA, which was confirmed by mass spectrometry.

FIG. 16 shows the sequence of Lys-gingipain (SEQ ID NO: 5) from the organism Porphyromonas gingivalis.

FIGS. 17A and 17B show the heavy and light chain amino acid sequences, respectively, of KB001 (which includes HC SEQ ID NO: 55 and LC SEQ ID NO: 56). The construct is a mouse construct, which can be used in any of the method embodiments provided herein.

FIG. 18 shows a graph of the dose response titration binding of KB001 monoclonal antibodies from various hybridoma clones to isolated P. gingivalis gingipains.

FIG. 19 shows non-limiting examples of sequences for a GST-Gingipain recombinant protein, the GST recombinant epitopes, and the Gp portion of the GST-Gingipain fusion proteins.

FIGS. 20A and 20C are sequence constructs used in the example provided herein to identify at least a part of the epitope for KB001.

FIG. 20B are gel and western blot results from HRgpA and Kgp (FIGS. 20A and 20C).

FIG. 21 Shows the results from an ELISA test demonstrating various tested fusion constructs, one is designated rGP-1 (see FIG. 4) and another is designated rGP-2 (see FIGS. 5A and 5B). The rGP-1 as comparable to the rGP-2. Also determined in this experiment is a concentration of 13.75 ng/well of recombinant GP was sufficient for coating. Shows the results from an ELISA test comparing the performance of recombinant proteins rGP-1 and rGP-2 as plate-coating proteins for analysis of anti-GP antibodies. Also determined in this experiment is that a concentration of 13.75 ng/well of rGP-1 or rGP-2 is sufficient for coating.

FIG. 22 depicts an ELISA test in which anti-GP antibodies are detected in clinical plasma samples.

FIG. 23 depicts a western blot analysis.

### DETAILED DESCRIPTION

Provided herein are antigenic peptides and/or proteins (“AP”) for raising an immune response against Porphyromonas gingivalis.

While a variety of APs are provided herein for vaccination, some of these APs are believed to be even more useful as, following mapping studies of an antibody to the P.g. target, it was found that P.g. infected periodontal patients made natural antibody responses directed to non-protective epitope(s) adjacent to the epitope, but not under the epitope. Thus, some of the APs provided herein represent a protected epitope(s) that humans do not make under natural infections. Such embodiments include those having sequences that comprise, consist, or consist essentially of YTYTVYRDGTKIK (SEQ ID NO: 6) and derivatives, variants and fragments thereof.

In some embodiments, the AP of YTYTVYRDGTKIK (SEQ ID NO: 6) and derivatives, variants and fragments thereof is superior over other constructs, as antigens comprising at least a portion of the above sequence appear in a repeat pattern that is present in the target, making them more prevalent and thus effective as a targeted vaccination molecule.

In some embodiments, the AP of YTYTVYRDGTKIK (SEQ ID NO: 6) and derivatives, variants and fragments thereof is superior over other APs, as this sequence (and sequences similar thereto) is present across a variety of the P.g. proteins, such as Kgp, RgpA, and HagA.

In some embodiments, the AP of YTYTVYRDGTKIK (SEQ ID NO: 6) and derivatives, variants and fragments thereof is superior over other APs, as antibodies that bind to this sequence appear to halt required processing of the P.g., in a manner for it to be effective against the host, and thus, raising an immune response against this sequence will raise antibodies that also bind to or block this sequence, and similarly halt the processing (cleavage) of the protein.

In some embodiments, the protein has one or more of the above properties or functions. In some embodiments, only the bare sequence is contemplated and one or more of the above need not be present.

Also provided are methods of treating and/or preventing periodontal infection or local and systemic inflammation by targeting P. gingivalis, e.g., surface OMV structures of P. gingivalis, using an AP as described herein. In some embodiments, a method of the present disclosure includes identifying a subject in need of treating a condition, disorder or disease associated with Porphyromonas gingivalis, and administering to the subject a therapeutically effective amount of an AP as disclosed herein, to allow for an innate immune response to be raised in a host to inactivate and reduce/eliminate the bacteria and its toxic OMVs, thus treating the various conditions, disorders or diseases. In some embodiments, the condition, disorder or disease is, without limitation, one or more of vascular disease (e.g., cardiovascular disease, atherosclerosis, coronary artery disease, myocardial infarction, stroke, and cardiac hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD age related macro-degeneration, cerebral and abdominal aneurysms, glioma, large vessel stroke C-IMT, microvascular defects and associated dementias (e.g.,Parkinson's), Peri-Implantitis and/or periodontal disease and/or associated bone loss, cognitive disorders (e.g., early, middle, or late dementia; Alzheimer's disease); regenerative and stem cell dysfunction; and age-related disorder.

Also provided herein are methods of preventing any one of the conditions, disorders, or diseases, as disclosed herein, by administering to a subject, e.g., a subject at risk of developing the condition, disorder, or disease, an effective amount of an AP of the present disclosure, to thereby prevent the condition, disorder, or disease or developing. As used herein, “prevent” includes reducing the likelihood of a future event occurring, or delaying the onset of a future event. In some embodiments, the AP may be used preventatively within the oral subgingival cavity to create a barrier, retardant, and/or non-colonizing effect by P. gingivalis, thereby preventing the bacteria from gaining access to the oral cavity, or reducing the likelihood thereof.

In some embodiments, the AP of the present disclosure can be effective in preventing the initial periodontal growth or recolonization by P. gingivalis in a subject to which the AP is administered. Without being bound to theory, the AP is an especially prominent protective epitope, so as to allow for a host to create an especially effective immune response to the bacteria leading to its inability to initially colonize the oral cavity/host and or eliminate it from the host.

In some embodiments, the AP contains a set of one or more paired cysteines.

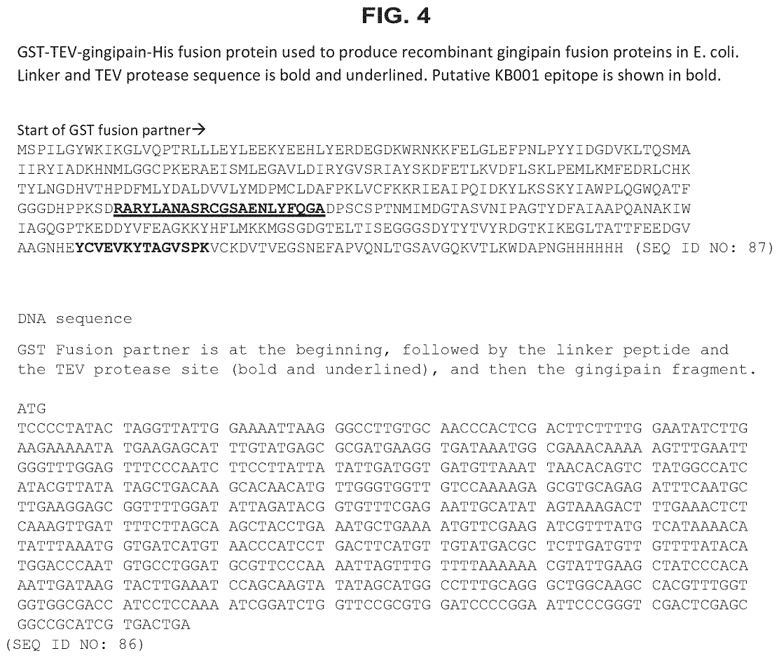

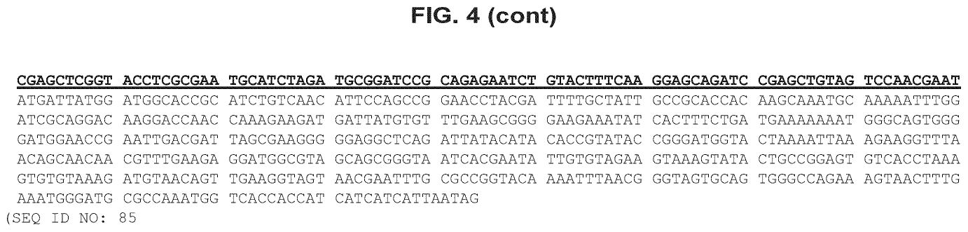

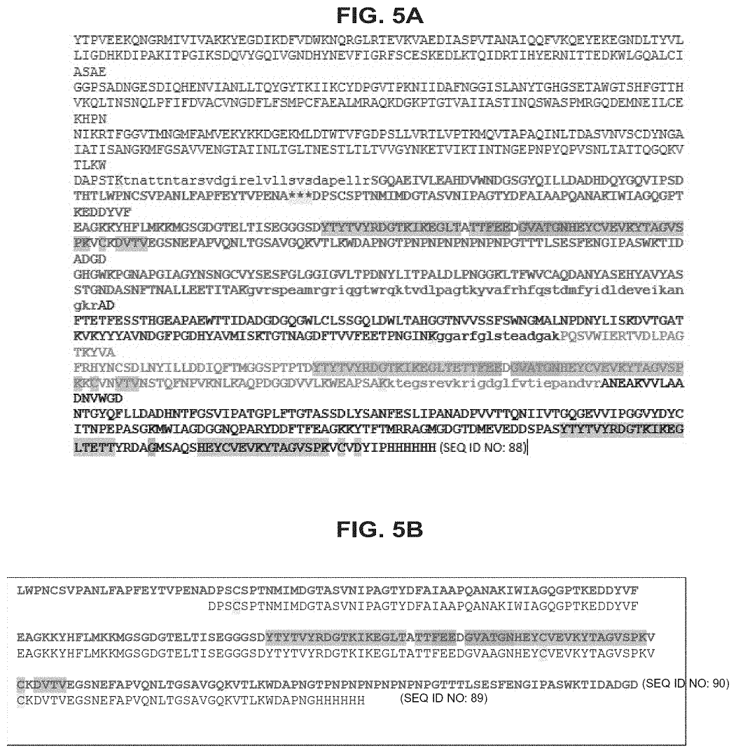

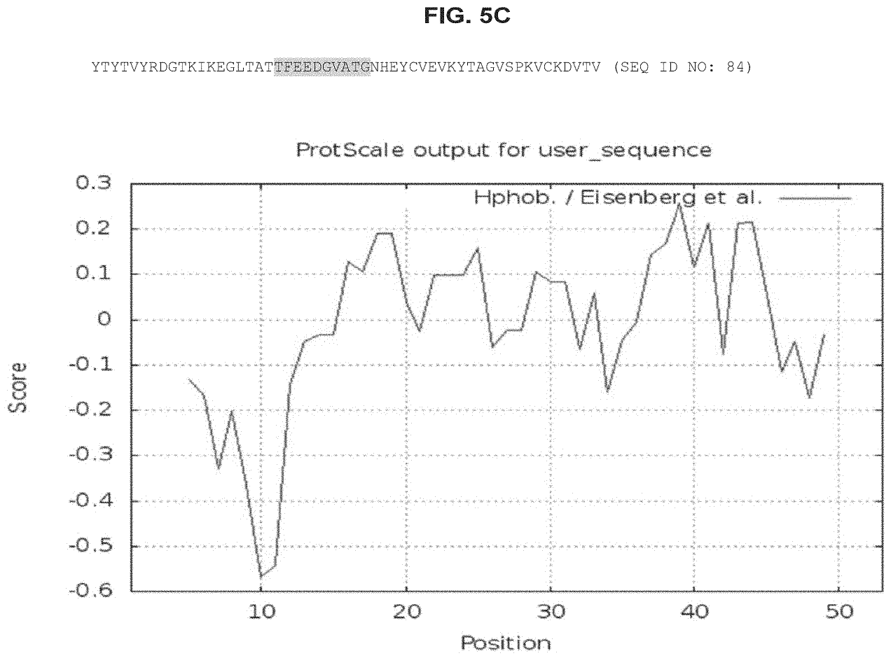

Any of the compositions (e.g., APs) provided herein can be exchanged with the constructs shown in FIGS. 4, 5A, 5B, and 5C and any protein fragment thereof, including those 99, 98, 97, 96, 95, 90, 85, 80, 75, 70, 60, 50, 40% similar or identical thereto for a vaccine application. In some embodiments, the AP comprises, consists, or consists essentially of YTYTVYRDGTKIK (SEQ ID NO: 6) and any protein fragment thereof, or any including those 99, 98, 97, 96, 95, 90, 85, 80, 75, 70, 60, 50, 40% similar or identical thereto for a vaccine application.

In some embodiments, the AP includes a Hag x-repeat, which is a motif that is present in various proteins/peptides of interest for gingipains. The motif comprises: YTYTVYRDGTKIK (SEQ ID NO: 6) as a component. The motif is present at least once in Pg, but in pre-processed forms of the protein, can be present multiple times (e.g., 2, 3, 4, 5, 6, 7, 8, 9 or 10, 11, 12, 13, 14, 15 times or more for various complexes). By employing an AP with this motif, it is believed that an especially large immune response can be developed. The motif can comprise longer sequences as well, such as items n-terminal and c-terminal to the YTYTVYRDGTKIK (SEQ ID NO: 6) sequence. In some embodiments, depending on Pg strain this motif is repeated at least twice on Kgp, 3× on RgpA and up to 6× on HagA. The sequence can occur at least 10 times on proteins associated with the Pg cell surface, making it superior for therapeutics. In some embodiments, variants of YTYTVYRDGTKIK (SEQ ID NO: 6) can be used, including those with 1, 2, 3, 4, 5, 6, 7, 8, 9, 10, or more additional amino acids of the parent protein (such as HagA, Kgp, or RgpA) and/or those with 1, 2, 3, 4, 5, 6, or more point mutations within the sequence (for example, using deletions, substitutions or additions to the sequence).

Also disclosed herein is an antigenic composition capable of raising an immune response against P. gingivalis in a subject. In some alternatives, the composition comprises an at least one isolated and purified peptide and/or protein, wherein the isolated and purified peptide and/or protein comprises an epitope that is bound by the antigen binding molecule KB001. In some embodiments, the peptide and/or protein comprises a sequence having at least about 75%, at least about 80%, at least about 85%, at least about 90%, at least about 95%, at least about 99%, and/or at about 100% identity to the sequence YTYTVYRDGTKIK (SEQ ID NO: 6). In some embodiments, the peptide and/or protein is not complexed with other Arg-specific and/or Lys-specific P. gingivalis proteinase and adhesin proteins.

Also disclosed herein is a composition for use in raising an immune response directed against P. gingivalis in a subject. In some embodiments, the subject is mammalian. In some embodiments, the subject is human.

As will be understood by one skilled in the art, the term “immune response” is given its standard scientific meaning and thus can refer to any detectable innate or adaptive response in a subject upon exposure to a stressor or pathogen. In some embodiments, the composition comprises an effective amount of at least one isolated and purified peptide and/or protein, wherein the isolated and purified peptide and/or protein comprises an epitope that is bound by the antigen binding molecule KB001. In some embodiments, the peptide and/or protein comprises a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a composition for use in the prevention and/or treatment of a disease or disorder arising from the infection of P. gingivalis in a subject. In some embodiments, the composition prevents one or more of the occurrence, duration, and/or symptoms arising from, and/or severity of the disease or disorder.

As will be understood by one skilled in the art, “infection” by P. gingivalis occurs when a subject has a detectable level of P. gingivalis in their system. Infection by P. gingivalis may therefore occur in any cell, tissue, organ, or organ system, including but not limited to the mouth, gums, blood, and bodily fluids. In some embodiments, the composition comprises an effective amount of an at least one isolated and purified peptide and/or protein, wherein the isolated and purified peptide and/or protein comprises an epitope that is bound by the antigen binding molecule KB001. In some embodiments, the peptide and/or protein comprises a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a composition for use as a therapeutic against a disease or disorder arising from the infection of P. gingivalis in a subject. In some embodiments, the composition has use in treating the symptoms arising from the disease or disorder. In some embodiments, the composition has use in treating the P. gingivalis infection directly. In some embodiments, the composition has use in the indirect treating of the disease or disorder caused by P. gingivalis infection. In some embodiments, the composition comprises an effective amount of an at least one isolated and purified peptide and/or protein, wherein the isolated and purified peptide and/or protein comprises an epitope that is bound by the antigen binding molecule KB001. In some embodiments, the peptide and/or protein comprises a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some embodiments, the peptide and/or protein comprises a sequence having at least about 75%, at least about 80%, at least about 85%, at least about 90%, at least about 95%, at least about 99%, and/or at about 100% identity to the sequence YTYTVYRDGTKIK (SEQ ID NO: 6). In some embodiments, the composition further comprises a pharmaceutically acceptable carrier. As will be understood by those skilled in the art, a “carrier” is given its usual scientific meaning and thus refers to any formulation that may improve one or more of the safety, effectiveness, and/or selectivity of the composition when administered to a subject. In some embodiments, the composition further comprises an adjuvant. As will be understood by those skilled in the art, an “adjuvant” is given its usual scientific meaning and thus refers to any substance that improves the subject's immune response to the composition. In some embodiments, the adjuvant is an aluminum salt adjuvant. In some embodiments, the composition is formulated for use orally. In some embodiments, the composition is formulated for percutaneous administration. In some embodiments, the composition is formulated for subcutaneous administration. In some embodiments, the composition is formulated for nasal administration. In some embodiments, the subject is mammalian and/or human. In some embodiments, the peptide and/or protein comprises one, two, three, and/or four sequences having at least about 75%, at least about 80%, at least about 85%, at least about 90%, at least about 95%, at least about 99%, and/or at about 100% identity to one, two, three, and/or all four of the sequences:

(a)
(SEQ ID NO: 1)
GVSPKVCKDVTVEGSNEFAPVQNLT,
(b)
(SEQ ID NO: 2)
YCVEVKYTAGVSPK,
(c)
(SEQ ID NO: 3)
AGTYDFAIAAPQANAKIWIAGQGPTKEDDYVFEAGKKYHFLMKKMGSGDG
TELTISEGGGSDYTYTVYRDGTKIKEGLTATTFEEDGVAAGNHEYCVEVK
YTAGVSPKVCKDVTVEGSNEFAPVQNLT,
and/or
(d)
(SEQ ID NO: 4)
GVSPK.

In some embodiments, the epitope is a conformational epitope defined by the sequence YTYTVYRDGTKIK (SEQ ID NO: 6). In some embodiments, the epitope is a conformational epitope defined by the sequence YTYTVYRDGTKIK (SEQ ID NO: 6), wherein a structure of the confirmational epitope is effectively the same as a structure of YTYTVYRDGTKIK (SEQ ID NO: 6) within a sequence of Kgp. In some embodiments, the epitope is a conformational epitope defined by the sequence AGTYDFAIAAPQANAKIWIAGQGPTKEDDYVFEAGKKYHFLMKKMGS GDGTELTIS EGGGSDYTYTVYRDGTKIKEGLTATTFEEDGVAAGNHEYCVEVKYTAGVSPKVCK DVTVEGSNEFAPVQNLT (SEQ ID NO: 3), wherein a structure of the confirmational epitope is effectively the same as a structure of AGTYDFAIAAPQANAKIWIAGQGPTKEDDYVFEAGKKYHFLMKKMGS GDGTELTIS EGGGSDYTYTVYRDGTKIKEGLTATTFEEDGVAAGNHEYCVEVKYTAGVSPKVCK DVTVEGSNEFAPVQNLT (SEQ ID NO: 3), within a sequence of Kgp. In some embodiments, the peptide and/or protein is of 50 or fewer amino acids. In some embodiments, the peptide and/or protein is of 49 or fewer amino acids. In some embodiments, the peptide is no longer than 40 amino acids in length. In some embodiments, the peptide is no longer than 30 amino acids in length. In some embodiments, the peptide is no longer than 20 amino acids in length. In some embodiments, the peptide is no longer than 15 amino acids in length. In some embodiments, the peptide is no longer than 14 amino acids in length. In some embodiments, the peptide is no longer than 10 amino acids in length.

Also disclosed herein is a method of treating a disease or condition associated with the presence of P. gingivalis in an oral tissue of a subject. As will be understood by one skilled in the art, “oral tissue” may refer to any cell or tissue type associated with the mouth and upper digestive tract. Non-limiting examples of oral tissue include the tongue, tongue cells, the gums, gum cells, lips, lip cells, hard palate cells, soft palate cells, the uvula, tonsil cells, tonsils, molars, and squamous epithelium cells. In some embodiments, the method comprises administering to a subject a composition disclosed herein and administering to the subject a peptide, wherein the polypeptide comprises: YTYTVYRDGTKIK (SEQ ID NO: 6), a polypeptide having at least 80% identity to this sequence, an immunogenic fragment of 8, 9, 10, 12, 13, or more consecutive amino acids of this sequence, and a variant comprising a conservative substitution of at least one amino acid of this sequence. In some embodiments, the immunogenic composition is administered at least two times. In some embodiments, the immunogenic composition is administered at least three times, at least four time, at least five times, at least ten times, and/or at least twenty times.

Also disclosed herein is a method of treating or preventing a vascular disease or symptoms thereof. In some embodiments, the method comprises identifying a subject in need of treating or preventing a vascular disease, or symptoms thereof, and administering to the subject a therapeutically effective amount of the immunogenic composition disclosed herein, thereby treating or preventing the vascular disease or symptoms thereof. In some embodiments, the vascular disease comprises cardiovascular disease, atherosclerosis, coronary artery disease, myocardial infarction, stroke, and myocardial hypertrophy. In some embodiments, the method further comprises administering to the subject at least one other therapeutic agent for treating or preventing the vascular disease, or symptoms thereof. In some embodiments, the other therapeutic agent comprises a serum lipid lowering agent. In some embodiments, the other therapeutic agent is a statin. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a method of treating or preventing a vascular disease or symptoms thereof. In some embodiments, the method comprises administering to a subject in need of treating or preventing a vascular disease, or symptoms thereof, a therapeutically effective amount of at least one therapeutic agent for treating or preventing the vascular disease, or symptoms thereof, and administering an effective amount of the immunogenic composition disclosed herein, to thereby enhance the therapeutic effect of the at least one therapeutic agent. In some embodiments, the other therapeutic agent comprises a serum lipid lowering agent. In some embodiments, the other therapeutic agent is a statin. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is method of treating or preventing a systemic disease or symptoms thereof. In some embodiments, the method comprises identifying a subject in need of treating or preventing a systemic disease or symptoms thereof, wherein the systemic disease is one or more of type II diabetes, insulin resistance and metabolic syndrome, and administering to the subject a therapeutically effective amount of the immunogenic composition disclosed herein, thereby treating or preventing the systemic disease or symptoms thereof. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a method of treating or preventing rheumatoid arthritis or symptoms thereof. In some embodiments, the method comprises identifying a subject in need of treating rheumatoid arthritis or symptoms thereof, and administering to the subject a therapeutically effective amount of the immunogenic composition disclosed herein, thereby treating or preventing the rheumatoid arthritis or symptoms thereof. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a method of treating or preventing cancer or symptoms thereof. In some embodiments, the method comprises identifying a subject in need of treating cancer or symptoms thereof, and administering to the subject a therapeutically effective amount of the immunogenic composition disclosed herein, thereby treating or preventing the cancer or symptoms thereof. In some embodiments, the cancer is oral, gastrointestinal, lung or pancreatic cancer. In some embodiments, the method further comprises administering to the subject at least one other therapeutic agent for treating or preventing the cancer, or symptoms thereof. In some embodiments, the other therapeutic agent comprises a small molecule drug or immunotherapeutic agent. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a method of treating or preventing cancer or symptoms thereof. In some embodiments, the method comprises administering to a subject in need of treating or preventing cancer, or symptoms thereof, a therapeutically effective amount of at least one therapeutic agent for treating or preventing the cancer, or symptoms thereof, and administering an effective amount of the immunogenic composition disclosed herein, to thereby enhance the therapeutic effect of the at least one therapeutic agent. In some embodiments, the at least one therapeutic agent comprises a small molecule drug or immunotherapeutic agent. In some embodiments, the cancer is oral, gastrointestinal, lung or pancreatic cancer. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a method of treating or preventing a gut microbiome-related disorder or symptoms thereof. In some embodiments, the method comprises identifying a subject in need of treating a gut microbiome-related disorder or symptoms thereof, and administering to the subject a therapeutically effective amount of the immunogenic composition disclosed herein, thereby treating or preventing the gut microbiome-related disorder or symptoms thereof. In some embodiments, the gut microbiome-related disorder comprises inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a method of treating or preventing a cognitive disorder or symptoms thereof. In some embodiments, the method comprises identifying a subject in need of treating a cognitive disorder or symptoms thereof, and administering to the subject a therapeutically effective amount of the immunogenic composition disclosed herein, thereby treating or preventing the cognitive disorder or symptoms thereof. In some embodiments, the cognitive disorder is Alzheimer's disease. In some embodiments, the cognitive disorder is early, middle or late dementia. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a method of treating or preventing an age-related or longevity-related disorder, or symptoms thereof. In some embodiments, the method comprises identifying a subject in need of treating an age-related or longevity-related disorder, and administering to the subject a therapeutically effective amount of the immunogenic composition disclosed herein, thereby treating or preventing the age-related or longevity-related disorder, or symptoms thereof. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a method of treating or preventing a post event myocardial hypertrophy or symptoms thereof. In some embodiments, the method comprises identifying a subject in need of treating or preventing a post event myocardial hypertrophy or symptoms thereof, and administering to the subject a therapeutically effective amount of the immunogenic composition disclosed herein, thereby treating or preventing the post event myocardial hypertrophy or symptoms thereof. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is method of treating a wound. In some embodiments, the method comprises identifying a subject in need of treating a wound; and administering to the subject a therapeutically effective amount of the immunogenic composition disclosed herein, whereby closure of the wound is enhanced, thereby treating the wound. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a method of treating or preventing an age-related macular degeneration (AMD) or symptoms thereof. In some embodiments, the method comprises identifying a subject in need of treating or preventing AMD or symptoms thereof, and administering to the subject a therapeutically effective amount of the immunogenic composition disclosed herein, thereby treating or preventing the AMD or symptoms thereof. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a method of treating or preventing an aneurysm or symptoms thereof. In some embodiments, the method comprises identifying a subject in need of treating or preventing an aneurysm or symptoms thereof, and administering to the subject a therapeutically effective amount of the immunogenic composition disclosed herein, thereby treating or preventing the aneurysm or symptoms thereof. In some embodiments, the aneurysm is a cerebral or abdominal aneurysm. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a method of treating or preventing a glioma or symptoms thereof. In some embodiments, the method comprises identifying a subject in need of treating or preventing a glioma or symptoms thereof, and administering to the subject a therapeutically effective amount of the immunogenic composition disclosed herein, thereby treating or preventing the glioma or symptoms thereof. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a method of treating or preventing a large vessel stroke C-IMT or symptoms thereof. In some embodiments, the method comprises identifying a subject in need of treating or preventing a large vessel stroke C-IMT or symptoms thereof, and administering to the subject a therapeutically effective amount of the immunogenic composition disclosed herein, thereby treating or preventing the large vessel stroke C-IMT or symptoms thereof. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a method of treating or preventing microvascular defects and associated dementias, or symptoms thereof. In some embodiments, the method comprises identifying a subject in need of treating or preventing microvascular defects and associated dementias, or symptoms thereof, and administering to the subject a therapeutically effective amount of the immunogenic composition disclosed herein, thereby treating or preventing the microvascular defects and associated dementias, or symptoms thereof. In some embodiments, the microvascular defects and associated dementias comprises microvascular defects Parkinson's. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a method of treating or preventing a peri-implantitis or symptoms thereof. In some embodiments, the method comprises identifying a subject in need of treating or preventing a peri-implantitis or symptoms thereof, and administering to the subject a therapeutically effective amount of the immunogenic composition disclosed herein, thereby treating or preventing the peri-implantitis or symptoms thereof. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is method of treating or preventing a renal disease or symptoms thereof. In some embodiments, the method comprises identifying a subject in need of treating or preventing a renal disease or symptoms thereof, and administering to the subject a therapeutically effective amount of the immunogenic composition disclosed herein, thereby treating or preventing the renal disease or symptoms thereof. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a method of treating or preventing a regenerative and stem cell dysfunction, or symptoms thereof. In some embodiments, the method comprises identifying a subject in need of treating or preventing a regenerative and stem cell dysfunction, or symptoms thereof, and administering to the subject a therapeutically effective amount of the immunogenic composition disclosed herein, thereby treating or preventing the regenerative and stem cell dysfunction, or symptoms thereof. In some embodiments, the peptide and/or protein administered comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

Also disclosed herein is a method of treating or preventing a condition, disorder or disease associated with a P. gingivalis infection, or symptoms thereof. In some embodiments, the method comprises identifying a subject in need of treating or preventing a condition, disorder or disease associated with a P. gingivalis infection, or symptoms thereof, and administering to the subject a therapeutically effective amount of the immunogenic composition disclosed herein, thereby treating or preventing the condition, disorder or disease associated with a P. gingivalis infection, or symptoms thereof. In some embodiments, the method further comprises administering the therapeutically effective amount of the immunogenic composition to treat the condition, disorder or disease associated with a P. gingivalis infection, or symptoms thereof. In some embodiments, the method further comprises administering the therapeutically effective amount of the immunogenic composition to prevent the condition, disorder or disease associated with a P. gingivalis infection, or symptoms thereof. In some embodiments, the condition, disorder or disease is associated with a local infection of P. gingivalis. In some embodiments, the condition, disorder or disease is associated with an oral infection of P. gingivalis. In some embodiments, the condition, disorder or disease is one or more of: vascular disease (e.g., cardiovascular disease, atherosclerosis, coronary artery disease, myocardial infarction, stroke, and myocardial hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD (age-related macular degeneration), cerebral and abdominal aneurysms, glioma, large vessel stroke C-IMT, microvascular defects and associated dementias (e.g., Parkinson's), Peri-Implantitis and/or periodontal disease and/or associated bone loss, cognitive disorders (e.g., early, middle, and/or late dementia; Alzheimer's disease); regenerative and stem cell dysfunction; and longevity or age-related disorder. In some embodiments, the administering comprises administering the immunogenic composition intravenously, subgingivally, intradermally, subcutaneously, intrathecally, or by nebulization.

Also disclosed herein is a use of an immunogenic composition disclosed herein, for treatment of a disorder associated with, caused by or complicated by P. gingivalis. In some embodiments, the disorder associated with, caused by or complicated by P. gingivalis is one or more of: vascular disease (e.g., cardiovascular disease, atherosclerosis, coronary artery disease, myocardial infarction, stroke, and myocardial hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD (age-related macular degeneration), cerebral and abdominal aneurysms, glioma, large vessel stroke C-IMT, microvascular defects and associated dementias (e.g., Parkinson's), Peri-Implantitis and/or periodontal disease and/or associated bone loss, cognitive disorders (e.g., early, middle, and/or late dementia; Alzheimer's disease); regenerative and stem cell dysfunction; and longevity or age-related disorder.

Also disclosed herein is a method of screening for a vaccine. In some embodiments, the method comprises providing an antibody that binds to a Porphyromonas gingivalis protein PG0495, determining an epitope to which the antibody binds, and providing a fragment of the Porphyromonas gingivalis protein PG0495 as a protein fragment. In some embodiments, the epitope is a linear epitope. In some embodiments, the epitope is a conformational epitope. In some embodiments, the fragment is provided in a great enough concentration to be therapeutically effective.

Also disclosed herein is a prokaryotic or eucaryotic cell, which comprises a recombinant polynucleotide consisting of nucleotides encoding the polypeptide of at least one of a polypeptide of YTYTVYRDGTKIK (SEQ ID NO: 6), a polypeptide having at least 80% identity to SEQ ID NO: 6, an immunogenic fragment of 8, 9, 10, 12, 13, or more consecutive amino acids of SEQ ID NO: 6, and a variant comprising a conservative substitution of at least one amino acid of SEQ ID NO: 6, wherein the polynucleotide is operatively linked to at least one regulatory element.

In some embodiments, any of the AP (immunogenic peptides etc provided herein) can be encoded in or provide as a nucleic acid sequence instead of direct administration of the amino acid peptide. Thus, any of the polypeptides or variants thereof, and uses thereof provided herein can be modified so that the peptide is actually the corresponding nucleic acid sequence that encodes for the polypeptide. Thus, a vector or other delivery system can include one or more nucleic acid sequences of the AP (such as a peptide and/or protein that comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6). In some embodiments, a nucleic acid sequence is provided that encodes for a peptide that comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some embodiments, the composition of any embodiment disclosed herein comprises at least one amino acid from HagA and/or gingipiain. In some embodiments, the protein, epitope, polypeptide, amino acid sequence, or immunogenic fragment comprises at least one additional amino acid from HagA and/or gingipiain. In some embodiments, the AP contains a set of one or more paired cysteines. In some embodiments, the sequence of the peptide is at least 40, 50, 60, 70, 80, 90, 95, 96, 97, 98, 99, or 100% identical to the sequence in FIG. 4 or 5A, 5B, or 5C. In some embodiments, the sequence of the peptide comprises at least YTYTVYRDGTKIK (SEQ ID NO: 6).

In some embodiments, the AP includes a) at least 1, 2, 3, 4, 5, 6, 7, 8, 9, 10, 11 or more of the amino acids within YTYTVYRDGTKIK (SEQ ID NO: 6), as well as b) optionally at least some additional P.g. sequence, and c) optionally some additional protein as a fusion construct, which together will be smaller than the size of the original P.g. protein, for example 90, 80, 70, 60, 50, 40, 30, 20, 10% or less in the number of amino acids present. In some embodiments, the total size of the AP will be less than 100 amino acids, such as 90, 80, 70, 60, 50, 40, 30, or 20 amino acids.

In some embodiments, the AP will include at least 1, 2, 3, 4, 5, 6, 7, 8, 9, 10, 11, 12, or all of the amino acids at any one or more of positions 1, 2, 3, 4, 5, 6, 7, 8, 9, 10, 11, 12, and/or 13 of YTYTVYRDGTKIK (SEQ ID NO: 6) (where the first Y is position 1 and the last K is position 13). Optionally, other residues from the various P.g. proteins can also be included in the construct.

In some embodiments, the AP comprises a polypeptide or peptide that includes at least 1, 2, 3, 4, 5, 6, 7, 8, 9, 10, 11, 12, or all of the amino acids at any one or more of positions 1, 2, 3, 4, 5, 6, 7, 8, 9, 10, 11, 12, and/or 13 of YTYTVYRDGTKIK (SEQ ID NO: 6) (where the first Y is position 1 and the last K is position 13), and can include up to the native P.g. protein in any one or more of SEQ ID Nos: 85-87, 90, 88-89, and 84, as shown in FIGS. 4 and 5A-5C. In some embodiments, the AP includes at least 1, 2, 3, 4, 5, 6, 7, 8, 9, 10, 11, 12, or all of the amino acids at any one or more of positions 1, 2, 3, 4, 5, 6, 7, 8, 9, 10, 11, 12, and/or 13 of YTYTVYRDGTKIK (SEQ ID NO: 6) (where the first Y is position 1 and the last K is position 13) and up to the KGP/RGP sequences in SEQ ID NO: 88, 89, and 84. In some embodiments, at least 20, 30, 40, 50, 60, 70, 80, 90, 95, 96, 97, 98, 99, or 100% of SEQ ID NO: 88, 89, and 84 is included in the AP with at least 1, 2, 3, 4, 5, 6, 7, 8, 9, 10, 11, 12, or all of the amino acids at any one or more of positions 1, 2, 3, 4, 5, 6, 7, 8, 9, 10, 11, 12, and/or 13 of YTYTVYRDGTKIK (SEQ ID NO: 6) (where the first Y is position 1 and the last K is position 13). Thus, in some embodiments, the AP includes at least part of the epitope contained within SEQ ID NO: 6, as well at a further section of the relevant protein, defined within any one of SEQ ID NO: 84, 88, and 89, but need not include all of such further section and, in some embodiments, includes no further portion of any of the native P.g. protein.

While various AP portions have been provided herein for a variety of differing embodiments, it is envisioned that APs having at least a portion of the sequence within SEQ ID NO: 6 are especially advantageous as APs against P.g. and can be superior over some of the other APs provided herein. As noted herein, the AP can further comprise additional sequence (e.g., from some or all of the sequences in FIGS. 4 and 5A-5C), and need not include all of the sequence in YTYTVYRDGTKIK (SEQ ID NO: 6). Variants of YTYTVYRDGTKIK (SEQ ID NO: 6) are also contemplated in this arrangement.

### Definitions

As used herein, an “epitope” can be formed both from contiguous amino acids, or noncontiguous amino acids juxtaposed by folding of a protein and or proteins of the same or different proteins. Epitopes formed from contiguous amino acids are typically retained on exposure to denaturing solvents, whereas epitopes formed by folding are typically lost on treatment with denaturing solvents. An epitope includes the unit of structure specifically bound by an immunoglobulin VH/VL pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein. In some embodiments, the epitope may have both linear and conformational sequence determinants and thus be derived from a single monomer, homo-dimer, homo trimer, etc., and/or hetero-dimers, hetero-trimers, etc. “Epitope” refers to the site on an antigen to which specific B cells and/or T cells respond so that antibody is produced. The immunogenic activity may be protective. The term “protective immunogenic activity” refers to the ability of a polypeptide to elicit an immunological response in a subject that prevents or inhibits infection by P. gingivalis.

In the case of the KB-001 epitope data demonstrates that the epitope is contained in part or whole in the repeat epitope in hemagglutinin/adhesion and HagA gingipains domain (RE-HagA). This means that the proteolytic protein/peptide products of the processed RE-HagA contains multiple copies of the epitope (1, possibly2, possibly, 3 possibly 4, or more).

The term “antigen” as used herein refers to a substance that is capable of stimulating immune responses. The immune responses stimulated by antigens may be one or both of humoral or cellular, and generally are specific for the antigen. An antigen is capable of initiating and mediating the formation of a corresponding immune body (antibody) when introduced into a subject. An antigen may possess multiple antigenic determinants such that the exposure of the subject to an antigen may produce a plurality of corresponding antibodies with differing specificities. Antigens may include, but are not limited to proteins, peptides, polypeptides, nucleic acids and fragments, variants and combinations thereof.

The terms peptides, proteins and polypeptides are used interchangeably herein.

An “isolated” polypeptide (or isolated protein) is one that has been removed from its natural environment. For instance, an isolated polypeptide is a polypeptide that has been removed from the cytoplasm or from the membrane of a cell, and many of the polypeptides, nucleic acids, and other cellular material of its natural environment are no longer present. An “isolatable” polypeptide is a polypeptide that could be isolated from a particular source. A “purified” polypeptide is one that is at least 60% free, preferably at least 75% free, and most preferably at least 90% free from other components with which they are naturally associated. Polypeptides that are produced outside the organism in which they naturally occur, e.g. through chemical or recombinant means, are considered to be isolated and purified by definition, since they were never present in a natural environment.

The term “surface accessible protein” refers to all surface exposed proteins, including for example, inner and outer membrane proteins, proteins adhering to the cell wall and secreted proteins.

As used herein, a “fragment” of a polypeptide preferably has at least about 20 residues, or 60 residues, or about 100 residues in length. Fragments of P. gingivalis polypeptides can be generated by methods known to those skilled in the art.

Optional or optionally means that the subsequently described event or circumstance can or cannot occur, and that the description includes instances where the event or circumstance occurs and instances where it does not. For example, the phrase, “optionally the composition can comprise a combination” means that the composition may comprise a combination of different molecules or may not include a combination such that the description includes both the combination and the absence of the combination (i.e., individual members of the combination).

Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologues, paralogs, fragments and other equivalents, variants, fragments, and analogs of the foregoing. As used in the present disclosure, it shall be appreciated that any designation of a peptide arrangement provided in the specification is also envisioned as a polypeptide arrangement in some embodiments (where all other variables or arrangements remain the same).

Amino acid substitutions in a native protein sequence may be “conservative” or “non-conservative” and such substituted amino acid residues may or may not be one encoded by the genetic code. A “conservative amino acid substitution” is one in which the amino acid residue is replaced with an amino acid residue having a chemically similar side chain (i.e., replacing an amino acid possessing a basic side chain with another amino acid with a basic side chain). A “non-conservative amino acid substitution” is one in which the amino acid residue is replaced with an amino acid residue having a chemically different side chain (i.e., replacing an amino acid having a basic side chain with an amino acid having an aromatic side chain). The standard twenty amino acid “alphabet” is divided into chemical families based on chemical properties of their side chains. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and side chains having aromatic groups (e.g., tyrosine, phenylalanine, tryptophan, histidine).

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The nucleic acid may be double stranded, single stranded, or contain portions of both double stranded or single stranded sequence. As will be appreciated by those in the art, the depiction of a single strand (“Watson”) also defines the sequence of the other strand (“Crick”). By the term “recombinant nucleic acid” herein is meant nucleic acid, originally formed in vitro, in general, by the manipulation of nucleic acid by endonucleases, in a form not normally found in nature. Thus an isolated nucleic acid, in a linear form, or an expression vector formed in vitro by ligating DNA molecules that are not normally joined, are both considered recombinant for the purposes of this disclosure. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the disclosure.

As used herein, “sequence identity” or “identity” in the context of two nucleic acid sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and, therefore, do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Any suitable means for making this adjustment may be used. This may involve scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may include additions or deletions (i.e., gaps) as compared to the reference sequence (which does not include additions or deletions) for optimal alignment of the two sequences. The percentage can be calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Any suitable methods of alignment of sequences for comparison may be employed. Thus, the determination of percent identity between any two sequences can be accomplished using a mathematical algorithm. Preferred, non-limiting examples of such mathematical algorithms are the algorithm of Myers and Miller, CABIOS, 4:11 (1988), which is hereby incorporated by reference in its entirety; the local homology algorithm of Smith et al, Adv. Appl. Math., 2:482 (1981), which is hereby incorporated by reference in its entirety; the homology alignment algorithm of Needleman and Wunsch, JMB, 48:443 (1970), which is hereby incorporated by reference in its entirety; the search-for-similarity-method of Pearson and Lipman, Proc. Natl. Acad. Sci. USA, 85:2444 (1988), which is hereby incorporated by reference in its entirety; the algorithm of Karlin and Altschul, Proc. Natl. Acad. Sci. USA, 87:2264 (1990), which is hereby incorporated by reference in its entirety; modified as in Karlin and Altschul, Proc. Natl. Acad. Sci. USA, 90:5873 (1993), which is hereby incorporated by reference in its entirety.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al., Gene, 73:237 (1988), Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994), which are hereby incorporated by reference in their entirety. The ALIGN program is based on the algorithm of Myers and Miller, supra. The BLAST programs of Altschul et al., JMB, 215:403 (1990); Nucl. Acids Res., 25:3389 (1990), which are hereby incorporated by reference in their entirety, are based on the algorithm of Karlin and Altschul supra.

As used herein, the terms “treat,” “treatment,” “treating,” or “amelioration” refer to therapeutic treatments, wherein the object is to reverse, alleviate, ameliorate, inhibit, slow down or stop the progression or severity of a condition, e.g., a chronic inflammatory condition, associated with a disease or disorder, e.g. arteriosclerosis, gingivitis, etc. The term “treating” includes reducing or alleviating at least one adverse effect or symptom of a condition, disease or disorder associated with, e.g., arteriosclerosis, gingivitis, etc. Treatment is generally “effective” if one or more local or systemic conditions, symptoms or clinical biomarkers of disease are reduced. Alternatively, treatment is “effective” if the progression of a disease is reduced or halted. That is, “treatment” includes not just the improvement of symptoms or biomarkers, but also a cessation of, or at least slowing of, progress or worsening of symptoms compared to what would be expected in the absence of treatment. Thus, a treatment is considered effective if one or more of the signs or symptoms of a condition described herein are altered in a beneficial manner, other clinically accepted symptoms are improved, or even ameliorated and/or reversed back to a more normal or normal state, or a desired response is induced e.g., by at least 10% following treatment according to the methods described herein. Beneficial or desired clinical results include, but are not limited to, alleviation of one or more symptom(s), diminishment of extent of disease, e.g., chronic inflammatory disease, stabilized (e.g., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

Efficacy of an agent, e.g., ABM, can be determined by assessing physical indicators of a condition or desired response, e.g. inflammation and/or infection. Efficacy can be assessed in animal models of a condition described herein, for example treatment of systemic chronic inflammatory diseases associated with an oral infection, e.g., periodontal disease. When using an experimental animal model, efficacy of treatment is evidenced when a statistically significant change occurs in one of a number of criteria, including a one or more biomarkers associated with inflammation following infection. In some embodiments, treatment according to the methods described herein can reduce the levels, and/or eliminate and/or prevent the colonization of the disease causing bacteria Porphyromonas gingivalis. In some embodiments, treatment according to the methods described herein can reduce the levels of a biomarker(s) or symptom(s) or the tissue pathology of a condition, e.g. infection or recolonization by at least 10%, at least 15%, at least 20%, at least 25%, at least 30%, at least 40%, at least 50%, at least 60%, at least 70%, at least 80%, at least 90% or more, at least 95% or more, at least 98% or more, at least 99% or more, or by about 100%.

The term “effective amount” as used herein refers to the amount of an active agent, e.g., AP, or composition needed to alleviate at least one or more criteria listed above of the disease or disorder, and relates to a sufficient amount of active agent or pharmacological composition to provide the desired effect. The term “therapeutically effective amount” therefore refers to an amount of active agent or composition that is sufficient to provide a particular anti-bacterial or anti-recolonization effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease.

As used herein, “subject” means a human or animal. The animal can be a vertebrate, including a mammal, such as a primate, dog or rodent. Primates include human, chimpanzees, cynomolgus monkeys, spider monkeys, and macaques, e.g., Rhesus. Rodents include mice, rats, woodchucks, ferrets, rabbits and hamsters. Animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a primate, e.g., a human. The terms, “individual,” “patient” and “subject” are used interchangeably herein.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. Delivery and/or placement options include any suitable medicament delivery systems for intraoral, interproximal, intrasulcular, intra-periodontal pocket, intracanal, and intranasal. In some embodiments, a suitable delivery option includes any suitable mechanical and automated dental and medical syringes, including all calibrated and non-calibrated, all attachments, and all designs of tips including but not limited to blunt ended, and side port; Medicament delivery trays and systems including Peri° Protect Trays; Medicament applicator delivery systems; Slow releasing medical preparation for intrasulcular drug delivery; Filler, oral packing, fiber, microparticles, films, gels, injectable gels, vesicular systems, strips compacts, chip, hydrogel, thermal gel, liquid, solid, including Actisite, Arestin, Atridox, Ossix Plus, Periochip, Periostat, Periofil; Injectable systems; Professional irrigation systems including piezoelectric and ultrasonic cavitron units with and without reservoir including Ora-Tec Viajet and Oral irrigation systems including Interplak, Waterpik, Hydrofloss, Viajet, Airflos_s and Pro.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is used herein to indicate a non-limiting example. Thus, “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-91 1910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

When the terms prevent, preventing, and prevention are used herein in connection with a given prophylactic treatment for a given condition (e.g., preventing infection by P. gingivalis), it is meant to convey that the treated subject either does not develop a clinically observable level of the condition at all, or develops it more slowly and/or to a lesser degree than he/she would have absent the treatment. These terms are not limited solely to a situation in which the subject experiences no aspect of the condition whatsoever. For example, a treatment will be said to have prevented the condition if it is given during exposure of a patient to a stimulus that would have been expected to produce a given manifestation of the condition, and results in the subject's experiencing fewer and/or milder symptoms of the condition than otherwise expected. A treatment can “prevent” infection by resulting in the subject's displaying only mild overt symptoms of the infection; it does not imply that there must have been no penetration of any cell by the infecting microorganism.

Similarly, reduce, reducing, and reduction as used herein in connection with the risk of infection with a given treatment (e.g., reducing the risk of a P. gingivalis infection) refers to a subject developing an infection more slowly or to a lesser degree as compared to a control or basal level of developing an infection in the absence of a treatment. A reduction in the risk of infection may result in the subject displaying only mild overt symptoms of the infection or delayed symptoms of infection; it does not imply that there must have been no penetration of any cell by the infecting microorganism (i.e., P. gingivalis).

When the terms treat and treating are used herein in connection with a given treatment for a given condition (e.g., treating an infection, or a disease (symptomatic infection) caused by P. gingivalis), it is meant to convey that the treated subject displays either no clinically observable level of the condition or displays it to a lesser degree than he did before the treatment. A treatment can “treat” an infection or disease by resulting in the subject displaying milder overt symptoms of the infection; it does not imply that there must have been a complete eradication of the infecting microorganism (i.e., P. gingivalis).

P. gingivalis

Porphyromonas gingivalis is a pathogen that converts the local and distant healthy microbiome of an individual into a disease-forming biofilm of both the mouth and gut. P. gingivalis has multiple survival mechanism, which creates a grossly undiagnosed chronic active/inactive infection in the host leading to a “silent” chronic state of systemic and end organ inflammation and ultimate failure.

The pathogen hypothesis for Alzheimer's disease has been met with new attention over the last 5 years, but the push back has been the Immune Privilege of the Brain and whether the suspected pathogen source is local or peripheral to the brain tissues. As appreciated herein, the effect of P. gingivalis in the brain is mostly if not entirely from an oral peripheral source. Second, new data from the largest analysis of AD brain tissues to date show no presence of P. gingivalis DNA in the brain. Thirdly, herein discovered is a one-of-a-kind virulent subunit of the primary suspected pathogen in the strategic sites of AD brain tissues. It is a unique subunit toxin “XXX Epitope” domain of P. gingivalis (which denotes a triple repeat in the polyprotein). This virulent subunit toxin plays a massive role in disrupting the NLRP3 inflammasome and the IL-1b pathways. IL-1b and ubiquinone have been shown to trigger the pathogenesis and progression of Alzheimer's disease. This same virulent subunit toxin plays an equally large role in systemic inflammation, immune disruption, and has disease-causing effects on basic human cellular biology. The delivery of the virulent toxin to the brain appears to be primarily vascular, with possibly additional access through neuronal, all however, occurring from the oral source of P. gingivalis. The data strongly suggests for the first time that the “XXX Epitope” and related material are coming to the brain in AD as secreted by outer membrane vesicles from the bacterial surface of oral cavities.

In some embodiments, the “XXX Epitope” comprises, consists, or consists essentially of at least a part of YTYTVYRDGTKIK (SEQ ID NO: 6), including fragments of 5, 6, 7, 8, 9, 10, 11, or 12 amino acids thereof. It is noted that unless designated otherwise, the reference to an AP sequence, such as SEQ ID NO: 6, denotes the linear sequence, the conformational sequence arrangement, both in the alternative and combined as options. For the sake of brevity, this designation applies to all APs provided herein, and shall be taken to denote all three options (only linear, only conformational, or both). Of course, for a conformational arrangement, the residues involved in raising an immune response need not be all residues within the sequence and can, in some embodiments, include residues outside of the sequence. Thus, it is envisioned that for conformational variants of an AP, such as YTYTVYRDGTKIK (SEQ ID NO: 6), only 1, 2, 3, 4, 5, 6, 7, 8, 9, 10 or more of the residues may be involved as an antigen in some embodiments.

P. gingivalis Polypeptides (Including Proteins) and Nucleic Acids and Compositions Thereof

Provided herein are antigenic peptides and/or proteins (AP or APs) that can, for example, be used to vaccinate a subject against P. gingivalis.

In some embodiments, the antigenic peptides, proteins, and/or antibodies disrupt the later stages of the major protein surface processing machinery and prevent the maturation of the unique subunit toxin “XXX Epitope.” This subunit toxin is needed for both P. gingivalis survival, and the creation of P. gingivalis's secreted outer membrane vesicles (OMVs) that result in systemic multi-systems pathology. The “XXX Epitope” is a one-of-a-kind virulent subunit protein complex in neuro-anatomic strategic sites of AD brain tissues. In some embodiments, the XXX Epitope denotes a repeat motif having one or more amino acids within the sequence of YTYTVYRDGTKIK (SEQ ID NO: 6).of YTYTVYRDGTKIK (SEQ ID NO: 6).

In some embodiments, the antigenic peptides and/or protein is a peptide that is bound by an antibody (e.g., an epitope). For instance, the antibody KB-001 is a monoclonal antibody with unique binding to P. gingivalis and its virulence factors, chief among those the “XXX Epitope” complex. KB-001 was shown during clinical study to prevent the recolonization of P. gingivalis, thereby eliminating all of the virulence factors of P. gingivalis contributing to systematic and/or organ-based inflammation at their source.

The KB-001 monoclonal antibody recognizes the proteinase/adhesin/hemagglutinating complex. As disclosed herein, the antibody recognized all 22 laboratory and 105 human clinical isolates strains and serotypes by IF. The immunogen used to generate the body was formalinized Porphyromonas gingivalis, strain W83 (full length protein). On a gel, KB-001 has multiple bands between 31 and 65 kDa, two bands around 14 kDa, and higher MW bands at around 113 kDa. It has a mouse isotype of IgG1, and is registered with the Entrez Gene ID 2552074 29256891 2551934.

The broader target activity of KB-00 is unusual with possible gene duplication(s) of critical accessory functions. The two arginine-specific gingipains, RgpA and RgpB, possess practically identical caspase-like catalytic domains and specifically cleave Arg-Xaa peptide bonds. RgpA, however, possesses a large C-terminal extension bearing a hemagglutinin-adhesion domain, which is absent from RgpB. The Rgp/Kgp/adhesion/hemagglutinins complex recognized by the antibody KB-001 include RgpA (Gingipain R1; also known as prpR1 or hemagglutinin HagE), Kgp (Lys-gingipain) and HagA (Hemagglutinin A) are responsible for the known major survival virulence factors that include colonization, agglutination, hemagglutination/heme acquisition via RBC lysis, amino acids, adhesion complex, and host defenses against innate complement degradation/inactivation and acquired immunity (antibody cleavage). The activity of RgpA, Kgp, and HagA are mediated through the human IL-1B/NLRP3 pathway, and thus binding of RgpA, Kgp, and/or HagA to KB-001 may also block the advancement and interaction of this cytokine with its receptors and downstream pathways, such as systematic cellular inflammation, host defenses, and pre-oncogenic pathways. Booth et al. showed that subgingical application of an anti-gingipain Al adhesin monoclonal antibody could prevent recolonization of subgingival plaque by P. gingivalis. As disclosed herein, the KB-001 antibody was mapped, and the inventors found that P.g. infected periodontal patients made natural antibody responses directed to non-protective epitope(s) adjacent to the KB-001 monoclonal antibody mapped epitope. Thus, the KB-001 antibody targets a protective epitope(s) that humans do not make under natural infections. Patients who had naturally developed a specific IgG1 and/or 4 response to the gingipains did not exhibit progressive disease, and appeared stable compared with those subjects with predominant IgG2/IgG3 responses.

Polypeptides suitable for use in the compositions described herein may be isolated or derived from the P. gingivalis strains. Preferred polypeptides comprise a fragment of at least 8, 9, 10, 12, 13, 16, 18, 20, 25, 30, 35, 40, 50, 60, 70, 80, 90, 100, 150, 200, 250 or more consecutive amino acids of any of the P. gingivalis sequences provided herein. In some embodiments, the APs are conformational epitopes from the noted protein of interest. In such situations, various embodiments are provided herein with a core sequence, and it will be understood that, in some embodiments, this core sequence (such as SEQ ID NO: 2) can further include other sections of the protein of interest for a larger conformational epitope. Thus, APs can be based on conformational epitopes as provided herein, with the core noted sequence providing part (or in other embodiments all) of the conformational epitope, and further amino acids providing additional structure.

In some embodiments, the AP is included in an antigenic composition. The composition can include at least one isolated and purified protein, wherein the isolated and purified protein consists of

(SEQ ID NO: 1)
GVSPKVCKDVTVEGSNEFAPVQNLT
and/or
(SEQ ID NO: 2)
YCVEVKYTAGVSPK,
(SEQ ID NO: 3)
AGTYDFAIAAPQANAKIWIAGQGPTKEDDYVFEAGKKYHFLMKKMGSGDG
TELTISEGGGSDYTYTVYRDGTKIKEGLTATTFEEDGVAAGNHEYCVEVK
YTAGVSPKVCKDVTVEGSNEFAPVQNLT,
and/or
(SEQ ID NO: 4)
GVSPK

or an immunogenic fragment or variant thereof.

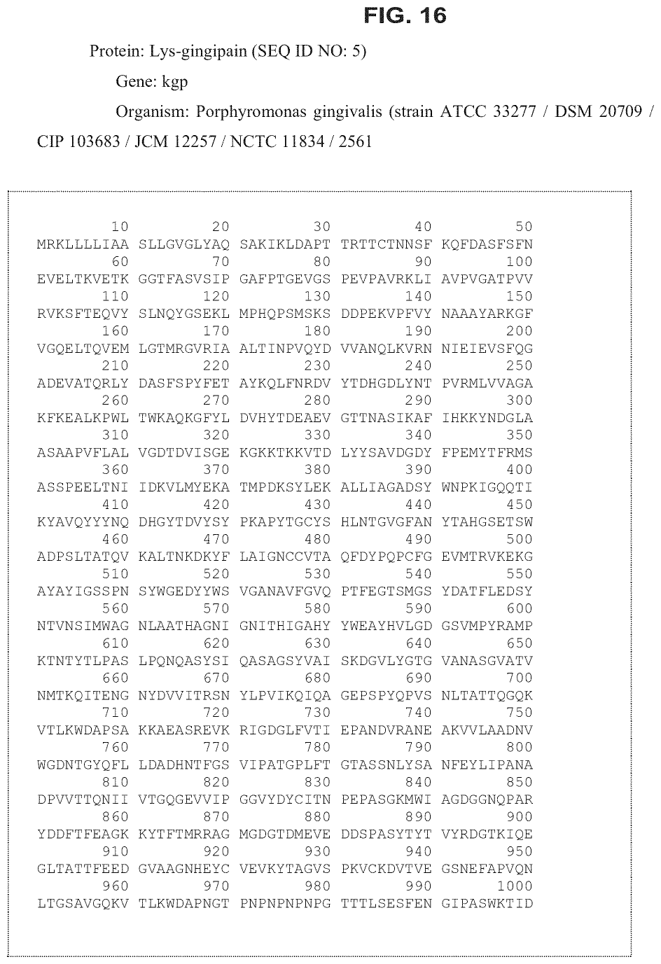

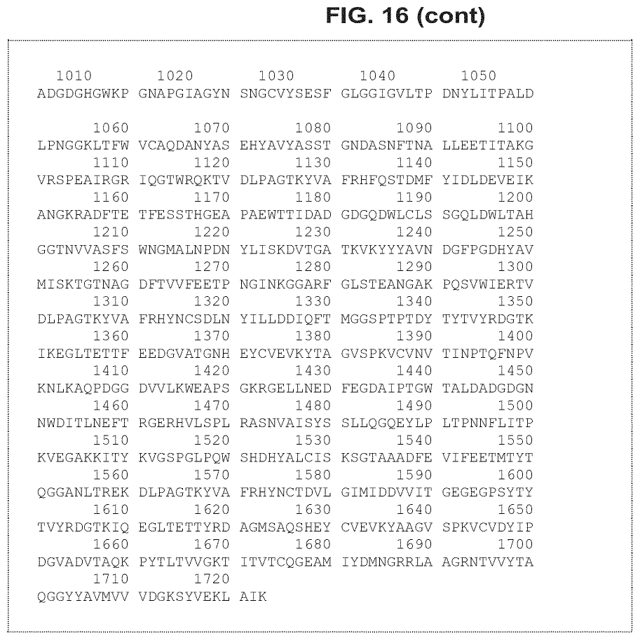

In some embodiments, the antigenic composition comprises a conformational epitope defined by the sequence YCVEVKYTAGVSPK (SEQ ID NO: 2), wherein a structure of the confirmational epitope is effectively the same as a structure of YCVEVKYTAGVSPK (SEQ ID NO: 2) within a sequence of Kgp. In some embodiments, the epitope can include additional sequences from Kgp. The sequence of the protein Lys-gingipainfrom the organism Porphyromonas gingivalis is as shown in FIG. 16.

While the present disclosure notes a variety of core sequences as part of a conformational epitope, it is noted that all of these sequences are also envisioned and therefore described herein in terms of the recited structures alone (and thus separate from any requirement of tertiary structure, apart from what it would inherently have in a pharmaceutically acceptable medium). For convenience and brevity, this is not explicitly spelled out in every statement herein, but should be understood as being disclosed as such.

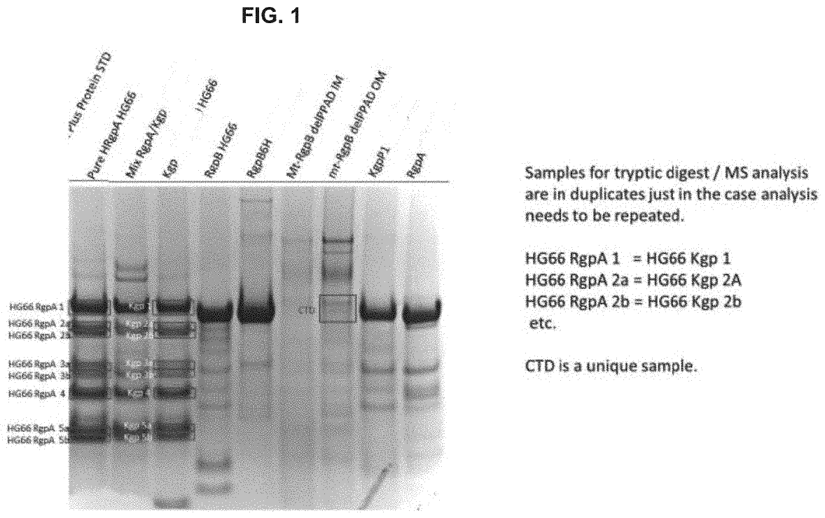

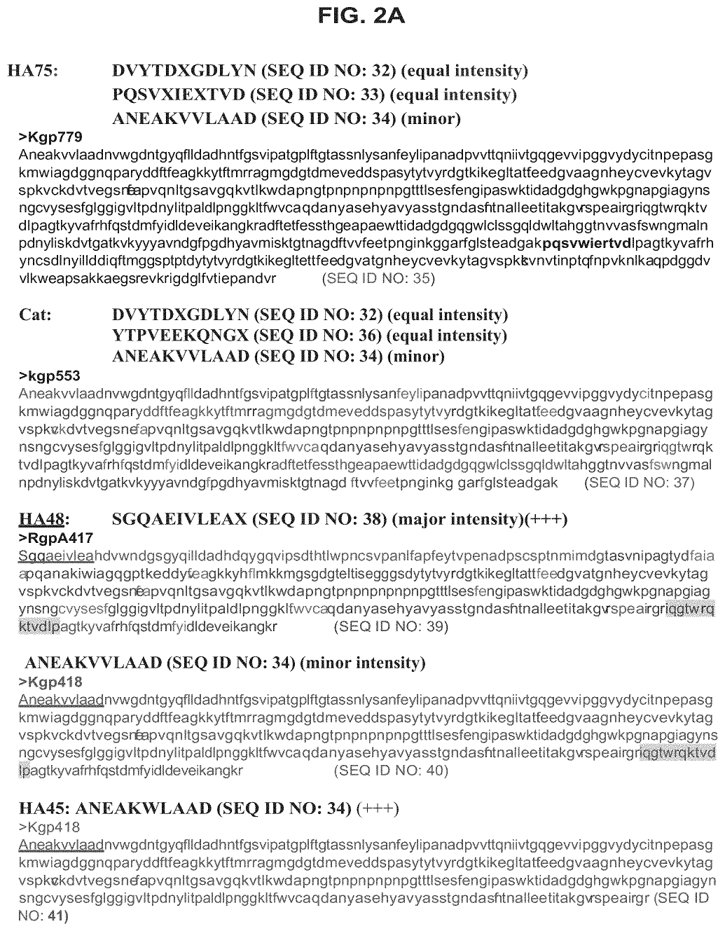

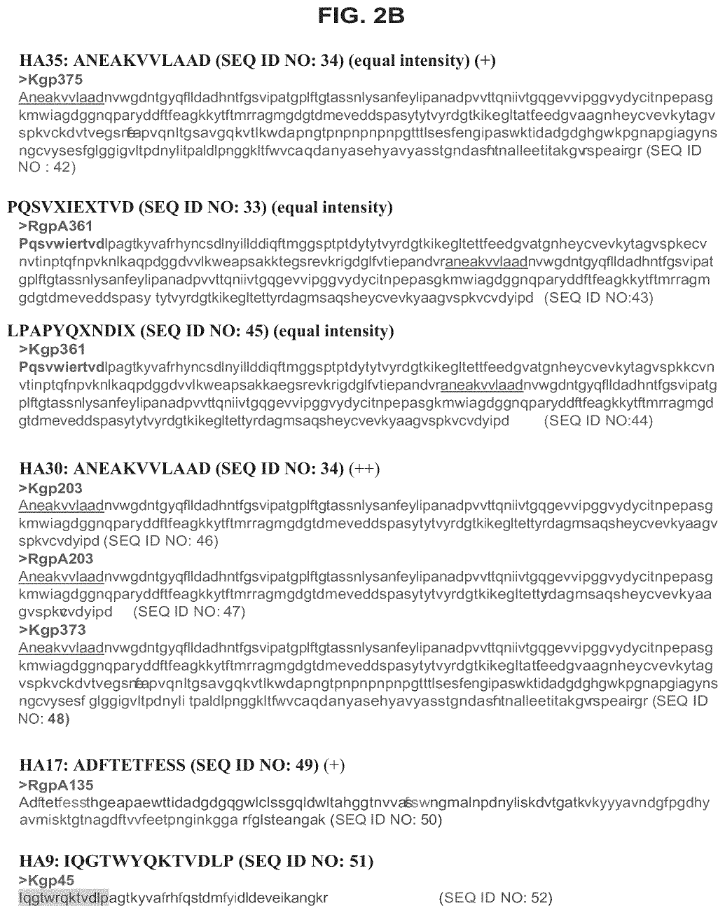

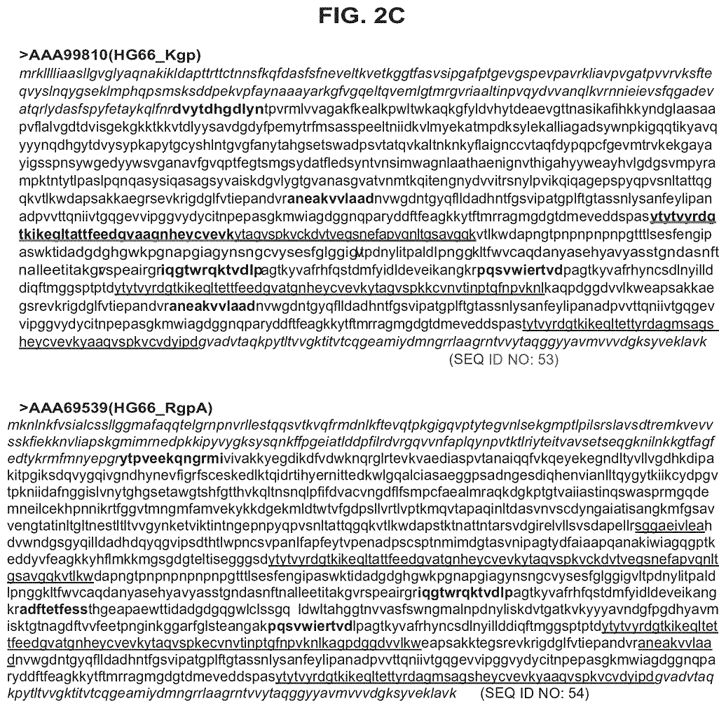

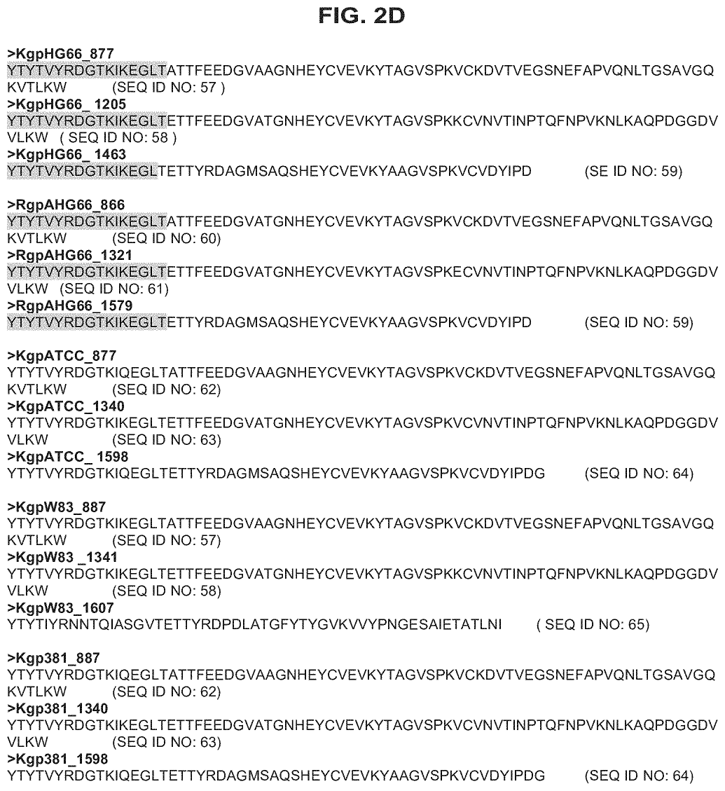

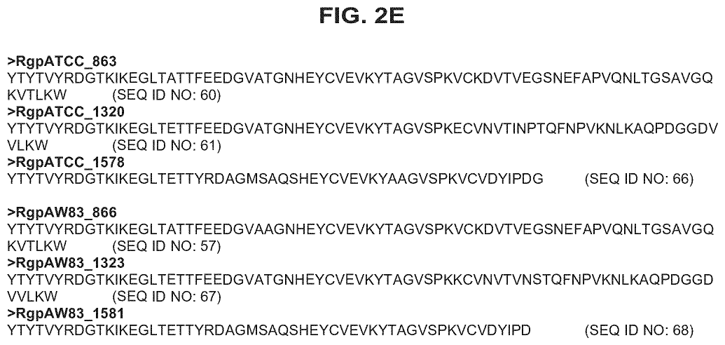

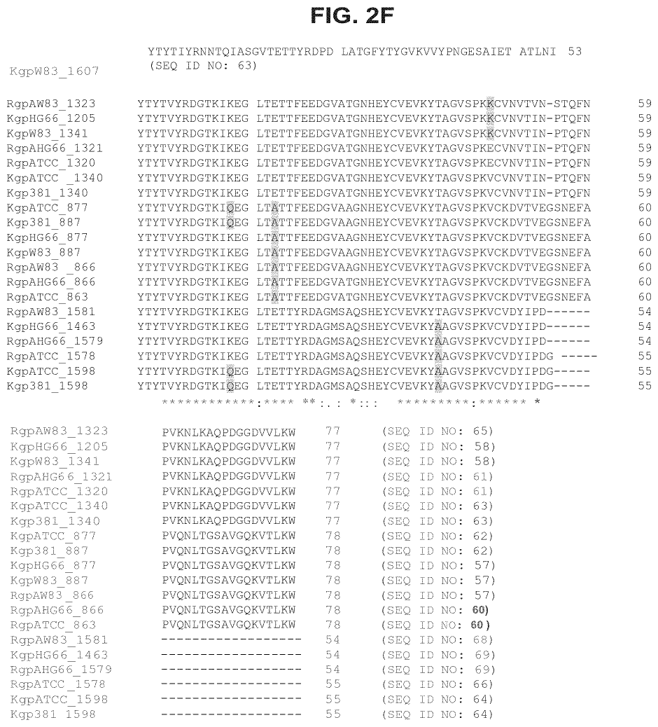

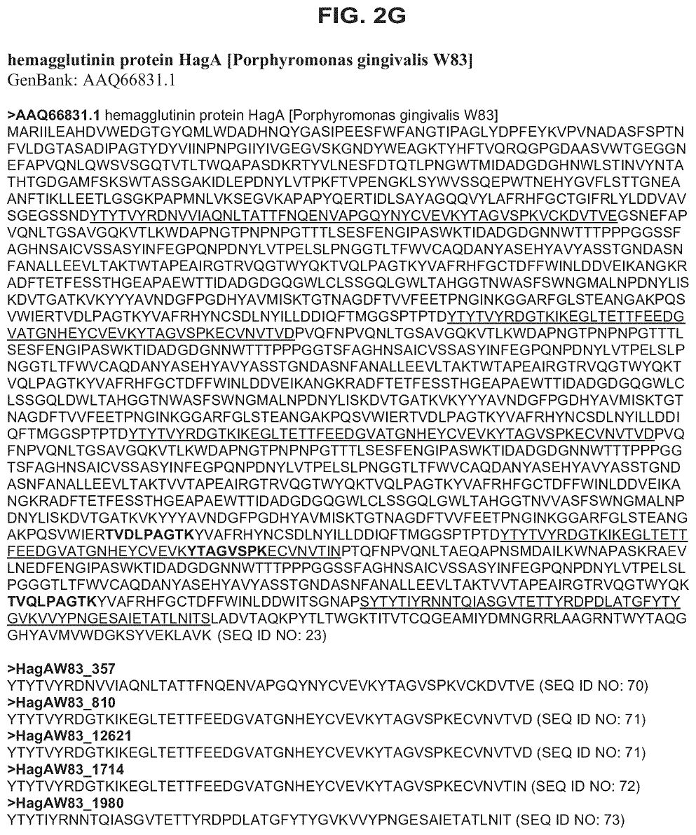

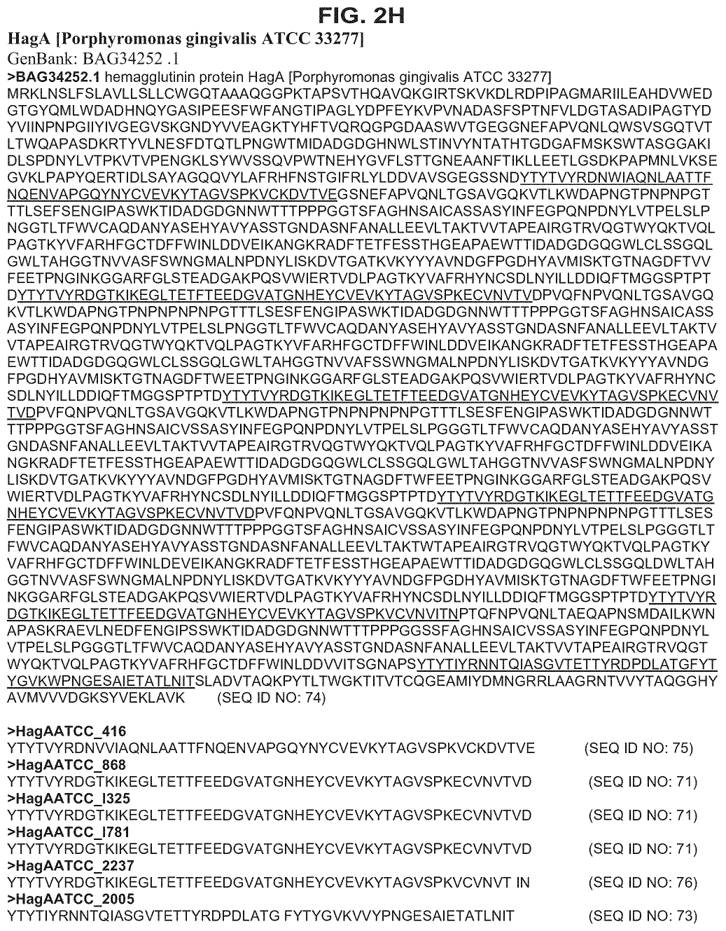

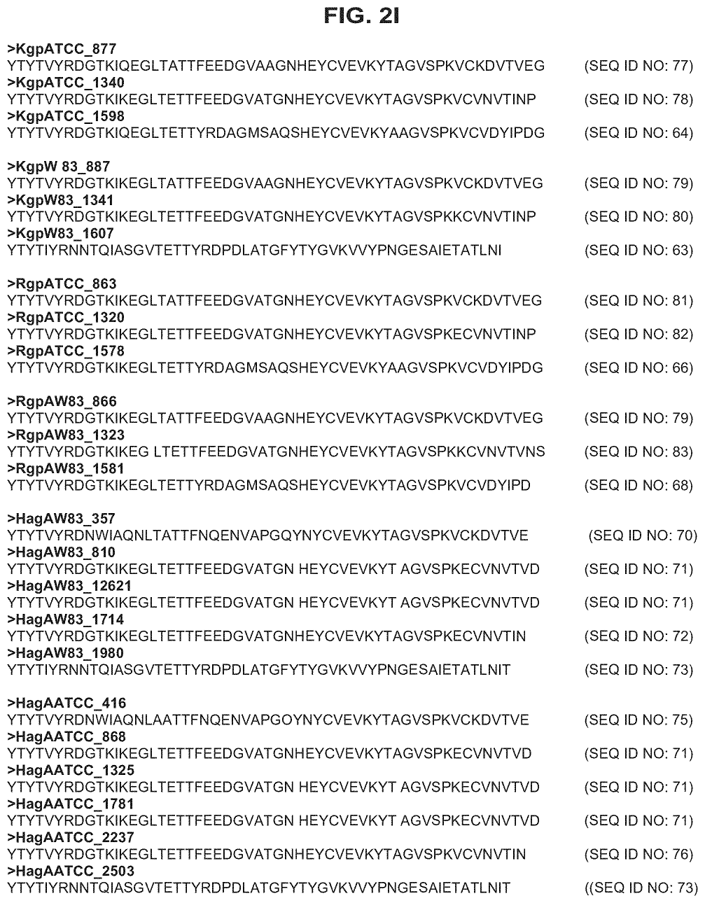

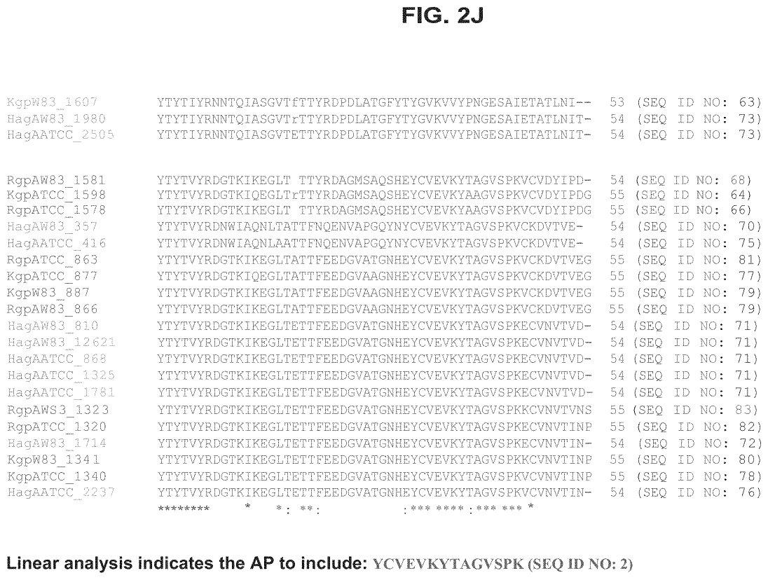

In some embodiments, an antigenic composition comprises a conformational epitope defined by the sequence GVSPKVCKDVTVEGSNEFAPVQNLT (SEQ ID NO: 1), wherein a structure of the confirmational epitope is effectively the same as a structure of GVSPKVCKDVTVEGSNEFAPVQNLT (SEQ ID NO: 1) within a sequence of Kgp. In some embodiments, the epitope can include additional sequences from Kgp. In some embodiments, the epitope can include additional sequences from Kgp. In some embodiments, the epitope sequence (and thus the relevant AP sequence) is that depicted (e.g., identified either explicitly as a standalone sequence or underlined or shaded in) in any one of FIGS. 1, 2A-2J, or some combination thereof. The data demonstrates that the epitope (and thus an appropriate AP, as provided herein) is contained in part or whole in the repeat epitope in hemagglutinin/adhesion and HagA gingipains domain (RE-HagA). This means that the proteolytic protein/peptide products of the processed RE-HagA contains multiple copies of the epitope (1, possibly 2, possibly 3, possibly 4, or more).

In some embodiments, an antigenic composition comprising a conformational epitope comprises one or more amino acid within GVSPK (SEQ ID NO: 4), wherein a structure of the confirmational epitope is effectively the same as a structure of a GVSPK fragment when located within YCVEVKYTAGVSPK (SEQ ID NO: 2). In some embodiments, the epitope can include additional sequences from Kgp.

In some embodiments, an antigenic composition comprising a conformational epitope comprises one or more amino acids within GVSPK (SEQ ID NO: 4), wherein a structure of the confirmational epitope is effectively the same as a structure of a GVSPK fragment when located within a full length sequence of Kgp. In some embodiments, the epitope can include additional sequences from Kgp.

In some embodiments, the antigenic composition comprises a conformational epitope defined by the sequence AGTYDFAIAAPQANAKIWIAGQGPTKEDDYVFEAGKKYHFLMKKMGS GDGTELTIS EGGGSDYTYTVYRDGTKIKEGLTATTFEEDGVAAGNHEYCVEVKYTAGVSPKVCK DVTVEGSNEFAPVQNLT (SEQ ID NO: 3), wherein a structure of the confirmational epitope is effectively the same as a structure of AGTYDFAIAAPQANAKIWIAGQGPTKEDDYVFEAGKKYHFLMKKMGS GDGTELTIS EGGGSDYTYTVYRDGTKIKEGLTATTFEEDGVAAGNHEYCVEVKYTAGVSPKVCK DVTVEGSNEFAPVQNLT (SEQ ID NO: 3), within a sequence of Kgp. In some embodiments, the epitope can include additional sequences from Kgp.

In some embodiments, an antigenic composition comprises or consists of a polypeptide of at least one of: a sequence of GVSPK (SEQ ID NO: 4), a sequence of GVSPK (SEQ ID NO: 4) having 1, 2, or 3 conservative substitutions, a sequence of GVSPK (SEQ ID NO: 4) having 1, or 2 substitutions, but having a similar structure to that of GVSPK when contained within a full length sequence of Kgp, a sequence YCVEVKYTAGVSPK (SEQ ID NO: 2), having at least 80% identity to SEQ ID NO: 2, an immunogenic fragment of 8, 9, 10, 12, 13, or more consecutive amino acids of SEQ ID NO: 2, and a variant comprising a conservative substitution of at least one amino acid of SEQ ID NO: 2, or a fragment thereof capable of raising an immune response against P. gingivalis, and wherein said polypeptide is not complexed with other Arg-specific and Lys-specific P. gingivalis proteinase and adhesin proteins. In some embodiments, the epitope formed can include additional sequences from Kgp.

In some embodiments, an isolated and purified polypeptide essentially consisting of a sequence YCVEVKYTAGVSPK (SEQ ID NO: 2) is provided as an AP. In some embodiments, this can be part of an epitope that can include additional sequences from Kgp.

In some embodiments, the AP comprises a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some embodiments, a composition for use in raising an immune response directed against P. gingivalis in a subject is provided. The composition comprises an effective amount of at least one amino acid sequence comprising at least 10 amino acids identical to a contiguous amino acid sequence of: 1) YCVEVKYTAGVSPK (SEQ ID NO: 2), 2) having at least 80% identity to SEQ ID NO: 2, 3) an immunogenic fragment of 8, 9, 10, 12, 13, or more consecutive amino acids of SEQ ID NO: 2, 4) a variant comprising a conservative substitution of at least one amino acid of SEQ ID NO: 2, and/or a peptide having the sequence having at least about 75%, at least about 80%, at least about 85%, at least about 90%, at least about 95%, at least about 99%, and/or at about 100% identity to the sequence YTYTVYRDGTKIK (SEQ ID NO: 6). and a pharmaceutically acceptable carrier. In some embodiments, the epitope formed by the AP can include additional sequences from Kgp.

In some embodiments, an immunogenic composition comprises: at least one isolated polypeptide of 49 or fewer amino acids, said polypeptide comprising an amino acid sequence selected from the group consisting of at least one of: 1) a polypeptide of YCVEVKYTAGVSPK (SEQ ID NO: 2), 2) a polypeptide having at least 80% identity to SEQ ID NO: 2, 3) an immunogenic fragment of 8, 9, 10, 12, 13, or more consecutive amino acids of SEQ ID NO: 2, 4) a variant comprising a conservative substitution of at least one amino acid of SEQ ID NO: 2, and/or 5) a peptide having the sequence having at least about 75%, at least about 80%, at least about 85%, at least about 90%, at least about 95%, at least about 99%, and/or at about 100% identity to the sequence YTYTVYRDGTKIK (SEQ ID NO: 6)., and an immunological adjuvant. In some embodiments, the epitope formed by the AP can include additional sequences from Kgp.

In some embodiments, an immunogenic composition comprises at least one isolated polypeptide, and a pharmaceutically acceptable carrier, wherein the at least one polypeptide is selected from the group consisting of Porphyromonas gingivalis protein YCVEVKYTAGVSPK (SEQ ID NO: 2), a polypeptide having at least 80% identity to SEQ ID NO: 2, an immunogenic fragment of 8, 9, 10, 12, 13, or more consecutive amino acids of SEQ ID NO: 2, and a variant comprising a conservative substitution of at least one amino acid of SEQ ID NO: 2. In some embodiments, the epitope formed can include additional sequences from Kgp.

In some embodiments, the additional sequences can be any surface exposed residue. In some embodiments, the additional sequences can be within 100, 90, 80, 70, 60, 50, 40, 30, 20, 15, 10, or 5 or fewer amino acids from the base protein (e.g. Kgp) of the noted core sequence (e.g., SEQ ID NO: 2). In some embodiments, the native protein sequence from which the AP is based is one that is only present (or primarily present) in emerging OMVs on the bacterial surface, in nature.

While some embodiments provided herein are derived from epitope information, it is to be noted that that need not be the case for all embodiments, as they can also simply be defined by the sequences recited herein and/or various structural and/or functional properties.

In some embodiments, the immunogenic composition can further comprise an adjuvant. In some embodiments, the adjuvant is an aluminum salt adjuvant.

In some embodiments, the immunogenic composition is formulated for use orally. In some embodiments, the immunogenic composition is formulated for percutaneous administration and/or transdermal. In some embodiments, the compound can be formulated for any route of administration to a human.

In some embodiments, an antigenic composition comprises at least one of: a polypeptide consisting essentially of GVSPK (SEQ ID NO: 4), a polypeptide of YCVEVKYTAGVSPK (SEQ ID NO: 2), a polypeptide having at least 80% identity to SEQ ID NO: 2, or an immunogenic fragment of 8, 9, 10, 12, 13, or more consecutive amino acids of SEQ ID NO: 2, and a variant comprising a conservative substitution of at least one amino acid of SEQ ID NO: 2. In some embodiments, the antigenic composition further comprises additional residues from Kgp.

In some embodiments, the antigenic composition includes a peptide that is no longer than 50 amino acids in length, for example, less than 50, 45, 40, 35, 30, 25, 20, 15, 14, 13, 12, 11, or 10 amino acids in length.

In some embodiments, the AP forms an epitope that includes the amino acid sequence GVSPKVCKDVTVEGSNEFAPVQNLT (SEQ ID NO: 1) (e.g., it can mimic the epitope to which any of the disclosed antibodies bind). In some embodiments, the peptide (peptides and/or proteins) of the AP includes an amino acid sequence at least about 70%, e.g., at least about 75%, at least about 80%, at least about 85%, at least about 90%, at least about 95%, at least about 97%, at least about 99%, or 100% identical to the sequence AGTYDFAIAAPQANAKIWIAGQGPTKEDDYVFEAGKKYHFLMKKMGS GDGTELTIS EGGGSDYTYTVYRDGTKIKEGLTATTFEEDGVAAGNHEYCVEVKYTAGVSPKVCK DVTVEGSNEFAPVQNLT (SEQ ID NO: 3). In some embodiments, the AP includes an amino acid sequence GVSPKVCKDVTVEGSNEFAPVQNLT (SEQ ID NO: 1), and includes an amino acid sequence at least about 70%, e.g., at least about 75%, at least about 80%, at least about 85%, at least about 90%, at least about 95%, at least about 97%, at least about 99%, or 100% identical to the sequence AGTYDFAIAAPQANAKIWIAGQGPTKEDDYVFEAGKKYHFLMKKMGS GDGTELTIS EGGGSDYTYTVYRDGTKIKEGLTATTFEEDGVAAGNHEYCVEVKYTAGVSPKVCK DVTVEGSNEFAPVQNLT (SEQ ID NO: 3). In some embodiments, the AP has the amino acid sequence GVSPKVCKDVTVEGSNEFAPVQNLT (SEQ ID NO: 1), and includes an amino acid sequence at least about 70%, e.g., at least about 75%, at least about 80%, at least about 85%, at least about 90%, at least about 95%, at least about 97%, at least about 99%, or 100% identical to residues 64-129 of the sequence AGTYDFAIAAPQANAKIWIAGQGPTKEDDYVFEAGKKYHFLMKKMGS GDGTELTIS EGGGSDYTYTVYRDGTKIKEGLTATTFEEDGVAAGNHEYCVEVKYTAGVSPKVCK DVTVEGSNEFAPVQNLT (SEQ ID NO: 3).

In some embodiments, the AP includes an amino acid sequence at least about 70%, e.g., at least about 75%, at least about 80%, at least about 85%, at least about 90%, at least about 95%, at least about 97%, at least about 99%, or 100% identical to residues 784 to 1130 of SEQ ID NO: 7.

In some embodiments, the AP includes the linear amino acid sequence YCVEVKYTAGVSPK (SEQ ID NO: 2). In some embodiments, the AP is or includes the epitope to which antibody KB-001 binds. In some embodiments, the AP includes the amino acid sequence YCVEVKYXIAGVSPK, where Xi is T or A. In some embodiments, the AP includes the amino acid sequence GVSPK (SEQ ID NO: 4).

In some embodiments, the AP includes the linear amino acid sequence of at least about 75%, at least about 80%, at least about 85%, at least about 90%, at least about 95%, at least about 99%, and/or at about 100% identity to the sequence YTYTVYRDGTKIK (SEQ ID NO: 6).

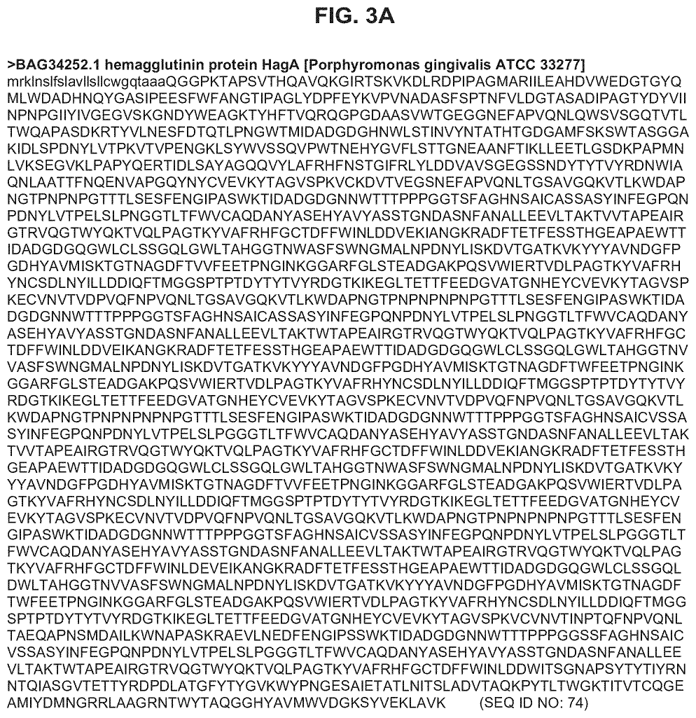

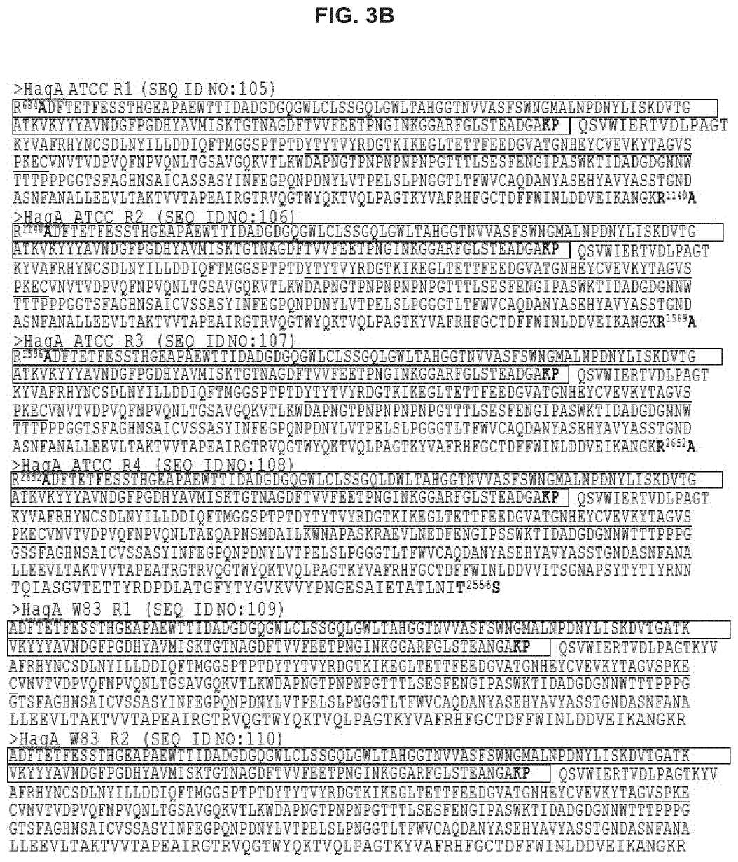

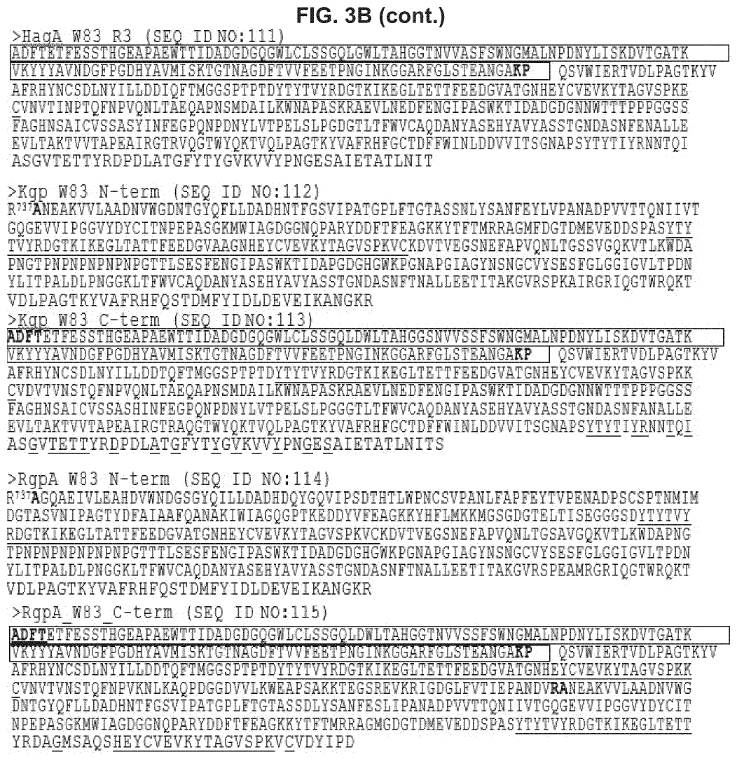

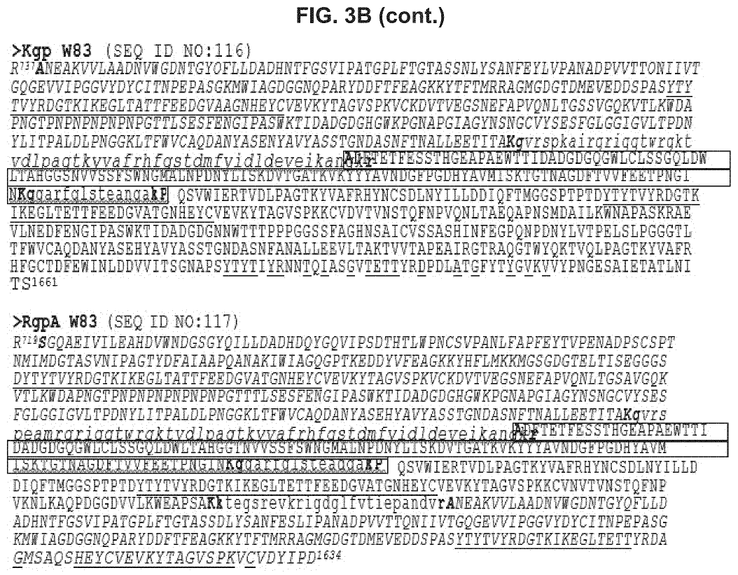

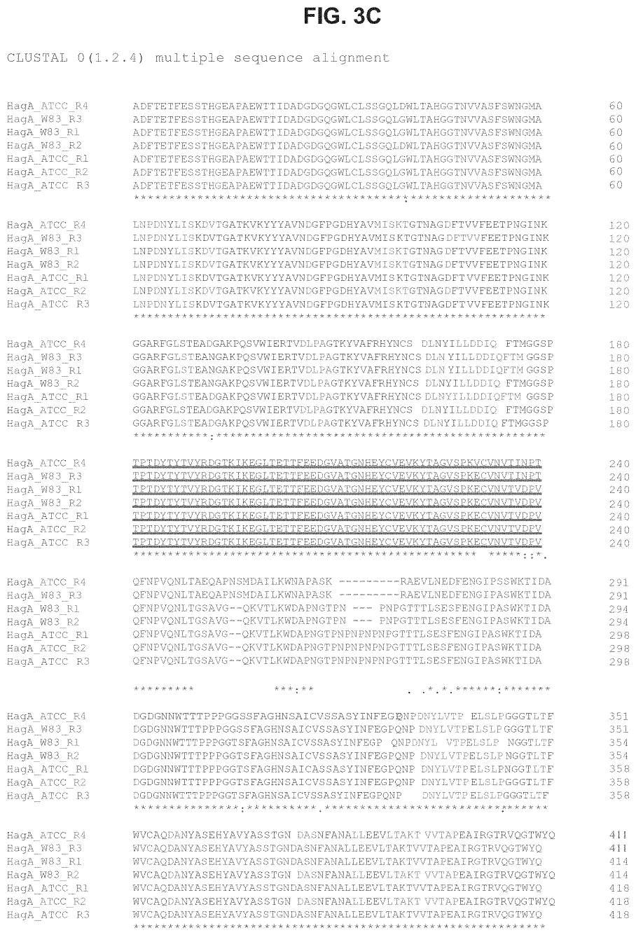

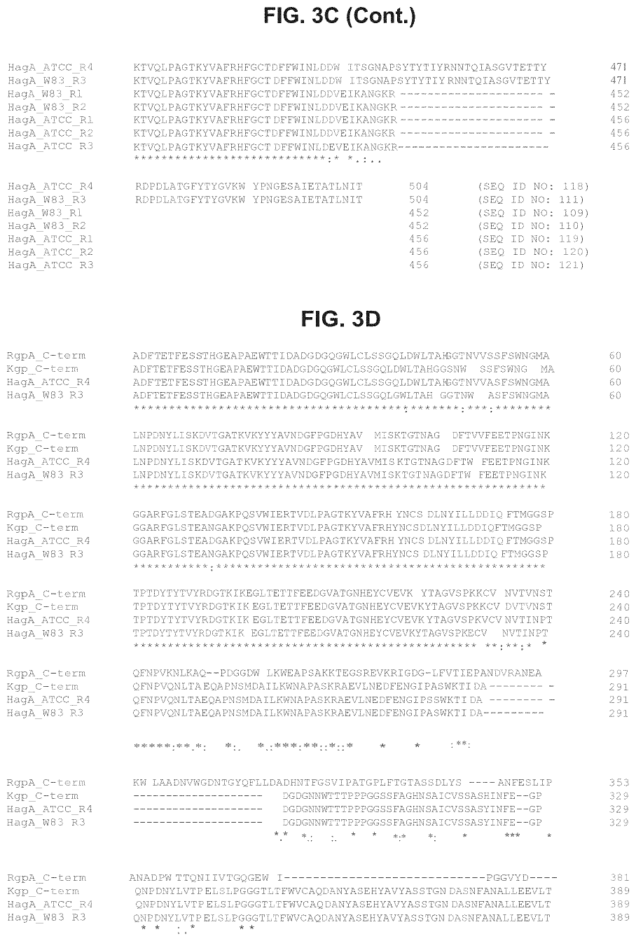

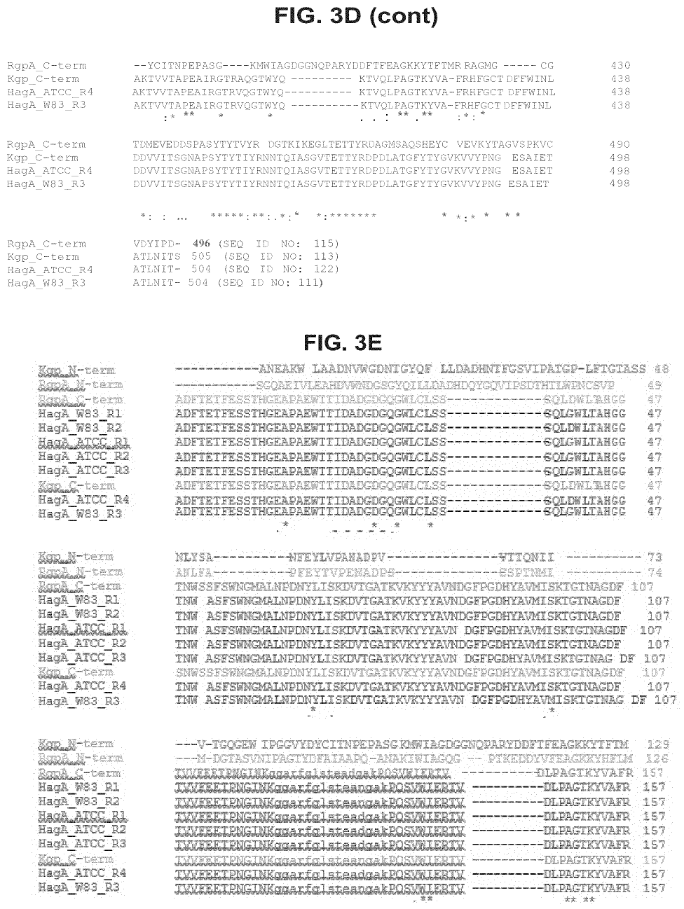

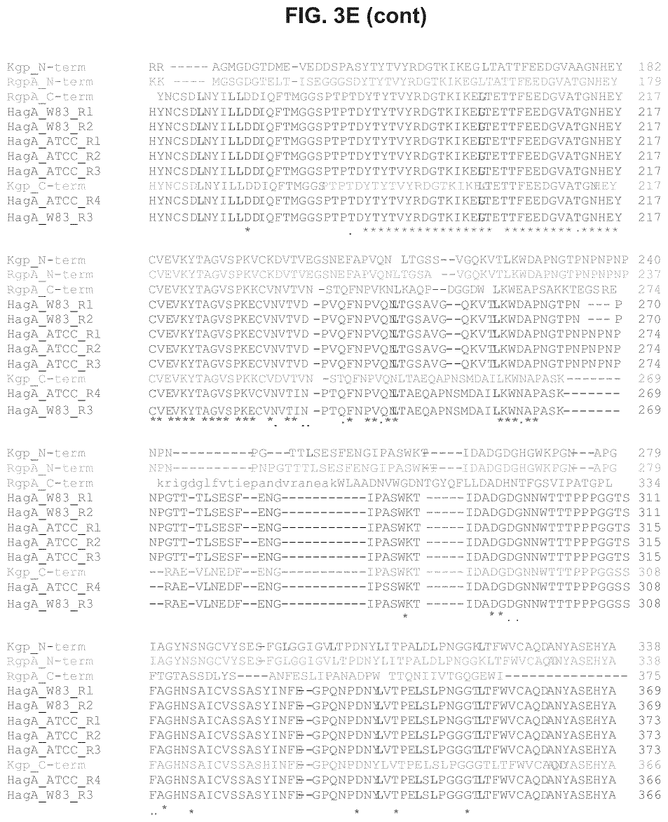

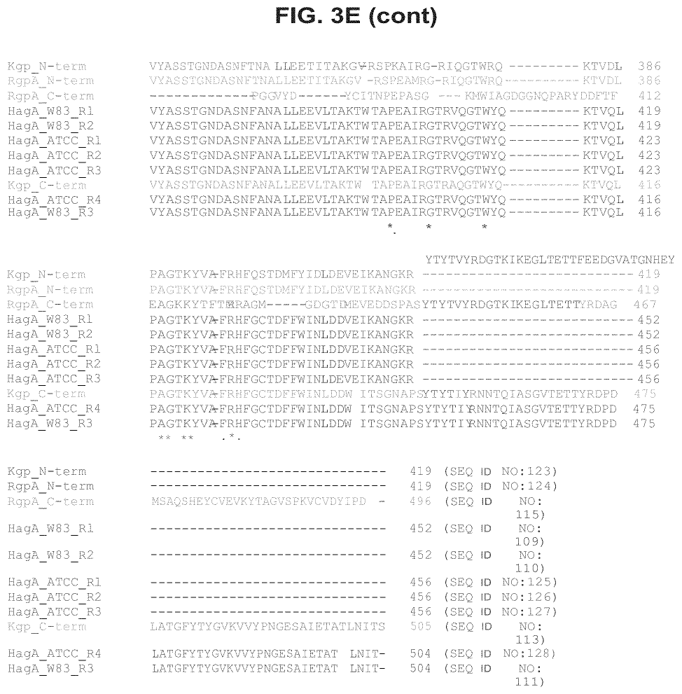

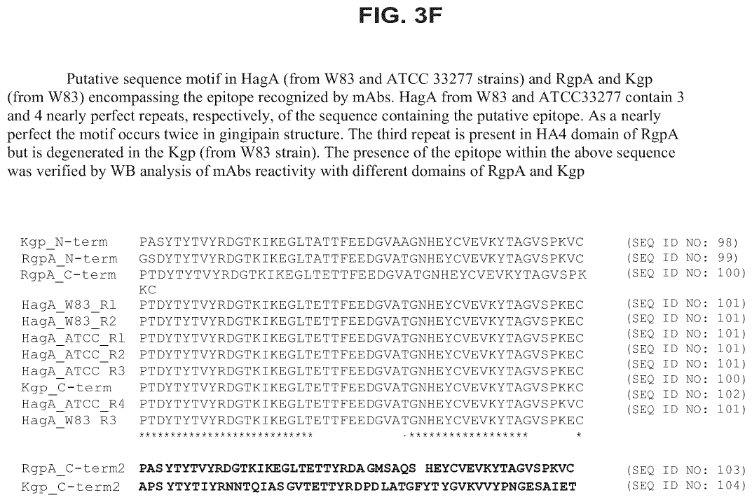

In some embodiments, the AP includes any of the above sequences, as well as a further sequence within a P. gingivalis gingipain (e.g., RgpA, Kgp) and/or hemagglutinin (e.g., HagA) from various strains. In some embodiments, the AP includes a sub-sequence of a P. gingivalis gingipain (e.g., RgpA, Kgp) and/or hemagglutinin (e.g., HagA) as shown in any one of FIGS. 3A-3F. FIG. 3B, provides non-limiting examples of amino acid sequences of the repeated domains of P. gingivalis gingipains and hemagglutinins (e.g., RgpA, Kgp, HagA) with sequences encompassing the various AP of the present disclosure underlined. The boxed portions indicate the HbR domain. Proteolytic processing sites are marked with bold font. In some embodiments, the AP includes a repeated domain of a P. gingivalis gingipain (e.g., RgpA, Kgp) and/or hemagglutinin (e.g., HagA). In some embodiments, the repeated domain containing the AP occurs at least 2, 3, 4 or more times within the P. gingivalis gingipain (e.g., RgpA, Kgp) and/or hemagglutinin (e.g., HagA). In some embodiments, HagA from W83 and ATCC33277, contains 3 and 4 nearly perfect repeats, respectively, of the sequence containing the putative epitope (FIGS. 3C, 3D, 3E, 3F) (and thus describing the core structure of the AP for some embodiments). In some embodiments, the motif containing the AP occurs twice in a gingipain structure (FIGS. 3D, 3E, 3F). In some embodiments, the third repeat is present in HA4 domain of RgpA but is degenerate in the Kgp (e.g., from W83 strain). In some embodiments, any one of these repeats can be used as all or part of the AP.

In some embodiments, the AP is part of or within any one of the amino acid sequences in Table 0.1. In some embodiments, the AP is or includes an amino acid sequence at least about 70%, e.g., at least about 75%, at least about 80%, at least about 85%, at least about 90%, at least about 95%, at least about 97%, at least about 99%, or 100% identical to any one of the amino acid sequences in Table 0.1. In some embodiments, the AP is bound by an antibody (e.g., KB-001). In some embodiments, the AP includes the sequence in Table 0.1 as well as some additional component or residue of RgpA, Kgp, and/or HagA.

TABLE 0.1
Putative Aps as motifs in HagA, RgpA and Kgp

| Source | | SEQ |
|---|---|---|
| (see Example 1) | Sequence | ID NO: |
| Kgp_N-term | PASYTYTVYRDGTKIKEGLTATTFEEDGVAAG | 8 |
| | NHEYCVEVKYTAGVSPKVC | |
| RgpA_N-term | GSDYTYTVYRDGTKIKEGLTATTFEEDGVATG | 9 |
| | NHEYCVEVKYTAGVSPKVC | |
| RgpA_C-term | PTDYTYTVYRDGTKIKEGLTETTFEEDGVATG | 10 |
| | NHEYCVEVKYTAGVSPKKC | |
| HagA_W83_R1 | PTDYTYTVYRDGTKIKEGLTETTFEEDGVATG | 11 |
| | NHEYCVEVKYTAGVSPKEC | |
| HagA_W83_R2 | PTDYTYTVYRDGTKIKEGLTETTFEEDGVATG | 12 |
| | NHEYCVEVKYTAGVSPKEC | |
| HagA_ATCC_R1 | PTDYTYTVYRDGTKIKEGLTETTFEEDGVATG | 13 |
| | NHEYCVEVKYTAGVSPKEC | |
| HagA_ATCC_R2 | PTDYTYTVYRDGTKIKEGLTETTFEEDGVATG | 14 |
| | NHEYCVEVKYTAGVSPKEC | |
| HagA_ATCC_R3 | PTDYTYTVYRDGTKIKEGLTETTFEEDGVATG | 15 |
| | NHEYCVEVKYTAGVSPKEC | |
| Kgp_C-term | PTDYTYTVYRDGTKIKEGLTETTFEEDGVATG | 16 |
| | NHEYCVEVKYTAGVSPKKC | |
| HagA_ATCC_R4 | PTDYTYTVYRDGTKIKEGLTETTFEEDGVATG | 17 |
| | NHEYCVEVKYTAGVSPKVC | |
| HagA_W83_R3 | PTDYTYTVYRDGTKIKEGLTETTFEEDGVATG | 18 |
| | NHEYCVEVKYTAGVSPKEC | |
| RgpA_C-term2 | PASYTYTVYRDGTKIKEGLTETTYRDAGMSAQ | 19 |
| | SHEYCVEVKYTAGVSPKVC | |
| Kgp_C-term2 | APSYTYTIYRNNTQIASGVTETTYRDPDLATGF | 20 |
| | YTYGVKVVYPNGESAIET | |

In some embodiments, the AP is a sequence from one or more P. gingivalis gingipains, where the gingipain is an arg-gingipain (Rgp) or a lys-gingipain (Kgp). In some embodiments, the AP is from (or includes a part of) one or more Rgps selected from RgpA and RgpB. In some embodiments, the AP is (or includes a part of) a RgpA sequence having an amino acid sequence at least about 80%, e.g., at least about 85%, at least about 90%, at least about 95%, at least about 97%, at least about 99%, or 100% identical to RgpA (SEQ ID NO: 7) (or some 20-100 amino acid fragment thereof). In some embodiments, the AP is (or includes a part of)a RgpB sequence having an amino acid sequence at least about 80%, e.g., at least about 85%, at least about 90%, at least about 95%, at least about 97%, at least about 99%, or 100% identical to SEQ ID NO: 21 (or some 20-100 amino acid fragment thereof).

In some embodiments, the AP is (or includes a part of) a peptide (e.g., peptides and/or proteins) having an amino acid sequence at least about 80%, e.g., at least about 85%, at least about 90%, at least about 95%, at least about 97%, at least about 99%, or 100% identical to SEQ ID NO:22 (or some 20-100 amino acid fragment thereof).

In some embodiments, the AP is (or includes a part of) a propeptide domain, a catalytic domain and/or a C-terminal adhesion domain of a gingipain. In some embodiments, the AP is (or includes a part of) a Rgp44 region of an RgpA adhesion domain, as described in, e.g., Li et al., Eur. J. Microbiol. Immunol., 2011, 1:41-58. In some embodiments, the AP is (or includes a part) of a Kgp39 region of a Kgp adhesion domain, as described in, e.g., Li et al., Eur. J. Microbiol. Immunol., 2011, 1:41-58.

In several embodiments, the AP includes a part of a P. gingivalis hemagglutinin/adhesin. In some embodiments, the hemagglutinin (or fragment thereof) is HagA. In some embodiments, HagA (or fragment thereof) has an amino acid sequence at least about 80%, e.g., at least about 85%, at least about 90%, at least about 95%, at least about 97%, at least about 99%, or 100% identical to SEQ ID NO:23. In some embodiments, the AP includes a part of an adhesion domain of HagA.

Preferred fragments comprise an AP as provided herein or conformational section thereof, with or without further components from the rest of the associated protein (e.g., Kgp, RpgB, or HagA). Other preferred fragments lack one or more acids from the N-terminus of the AP provided herein (e.g., 1, 2, 3, 4, 5, 6, 7, 8, 9, 10, 15, 20, 25 or more) and/or one or more amino acids from the C-terminus of the AP.

In some embodiments, provided herein are polynucleotides that encode a polypeptide (e.g., any of the APs or the sequences in AP compositions) as provided herein and polynucleotides which hybridize, under standard hybridization conditions, to a polynucleotide that encodes a polypeptide as provided herein, and the complements of such polynucleotide sequence. Also included are polynucleotides having sequence identity of at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity to a disclosed reference nucleic acid sequence (such as by reference to an amino acid sequence). The nucleic acids as provided herein, isolated or synthesized in accordance with the sequences disclosed herein are useful in the recombinant production of P. gingivalis peptides or polypeptides.

The nucleic acids can be obtained directly from the DNA of a P. gingivalis strain (such as for example, but not limited to, P. gingivalis strains W83, W50 and ATCC33277) and any other P. gingivalis strain carrying the applicable DNA gene sequence by using the polymerase chain reaction (PCR) (as described in PCR, A Practical Approach” (McPherson, Quirke, and Taylor, eds. IRL Press Oxford, UK, 1991) or by using alternative standard techniques that are recognized by one skilled in the art. The various embodiments encompass sequence-conservative variants and function-conservative variants of these sequences.

The polypeptides provided herein encompass those encoded by the disclosed isolated nucleic acids and their variants. The polypeptides as provided herein, including function-conservative variants, preferably correspond to proteins which are surface accessible on P. gingivalis.

The polypeptides as provided herein can be produced using standard molecular biology techniques and expression systems (see for example, Molecular Cloning: A Laboratory Manual, Third Edition by Sambrook et. al., Cold Spring Harbor Press, 2001). For example, the gene (or the fragment of a gene) that encodes an immunogenic polypeptide may be isolated and the polynucleotides encoding the immunogenic polypeptide may be cloned into any commercially available expression vector (such as, e.g., pBR322 and pUC vectors (New England Biolabs, Inc., Ipswich, Mass.) or expression/purification vectors (such as e.g., GST fusion vectors (Pfizer, Inc., Piscataway, N.J., or those described in the Examples herein) and then expressed in a suitable prokaryotic, viral or eukaryotic host. Purification may then be achieved by conventional means, or in the case of a commercial expression/purification system, in accordance with manufacturer's instructions.

Alternatively, the peptides and/or proteins as provided herein, including variants, may be isolated for example, but without limitation, from wild-type or mutant P. gingivalis cells, or through chemical synthetization using commercially automated procedures, such as for example, exclusive solid phase synthesis, partial solid phase methods, fragment condensation or solution synthesis.

Polypeptides as provided herein (in particular the APs) can have immunogenic activity. “Immunogenic activity” refers to the ability of a polypeptide to elicit an immunological response in a subject. An immunological response to a polypeptide is the development in a subject of a cellular and/or antibody-mediated immune response to the polypeptide. Usually, an immunological response includes but is not limited to one or more of the following effects: the product of antibodies, B cells, helper T cells, suppressor T cells and/or cytotoxic T cells, directed to an epitope or epitodes of the polypeptide.

A polypeptide as provided herein may be characterized by molecular weight, mass fingerprint, amino acid sequence, nucleic acid sequence that encodes the polypeptide, immunological activity, or any combination of two or more such characteristics. The molecular weight of a polypeptide, typically expressed in kilodaltons (kDa), can be determined using routine methods including, for instance, gel filtration, gel electrophoresis including sodium dodecyl sulfate (SDS) polyacrylamide gel electrophoresis (PAGE), capillary electrophoresis, mass spectrometry, liquid chromatography (including HPLC), and calculating the molecular weight from an observed or predicted amino acid sequence.

The immunogenic peptides and/or proteins of the various APs described herein include immunogenic fragments and variants of such peptides and/or proteins and/or fragments. Variants may comprise amino acid modifications. For example, amino acid sequence modifications include substitutional, insertional or deletion changes. Substitutions, deletions, insertions or any combination thereof may be combined in a single variant so long as the variant is immunogenic. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal or one or more amino acid residues from the protein sequence. Typically no more than about from 2 to 6 residues are deleted at any one site within the protein molecule. These variants ordinarily are prepared by site specific mutagenesis of nucleotides in the DNA encoding the protein, thereby producing DNA encoding the variant and thereafter expressing the DNA in a recombinant cell culture.

Techniques for making substitutional mutations at predetermined sites in DNA having a known sequence are well known and include, but are not limited to, M13 primer mutagenesis and PCT mutagenesis. Amino acid substitutions are typically single residues but can occur at a number of different locations. Substitutional variants are those in which at least one residue has been removed and a different residue inserted in its place. Such substitutions generally are made in accordance with the following Table 0.2 and are referred to as conservative substitutions (although non-conservative substitutions are also possible). Others are well known to those of skill in the art.

Conservative amino acid substitutions may involve a substitution of a native amino acid residue with a non-native residue such that there is little or no effect on the size, polarity, charge, hydrophobicity, or hydrophilicity of the amino acid residue at that position and, in particular, does not result in decreased immunogenicity. Suitable conservative amino acid substitutions are shown in Table 0.2.

TABLE 0.2

| Original | Exemplary | Preferred |
|---|---|---|
| Residues | Conservative Substitutions | ConservativeSubstitution |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, | Leu |
| | Norleucine | |
| Leu | Norleucine, Ile, Val, Met, Ala, | Ile |
| | Phe | |
| Lys | Arg, 1,4 Diamino-butyric Acid, | Arg |
| | Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

The specific amino acid substitution selected may depend on the location of the site selected. In certain embodiments, nucleotides encoding polypeptides and/or fragments substituted based on the degeneracy of the genetic code (i.e, consistent with the “Wobble” hypothesis). Where the nucleic acid is a recombinant DNA molecule useful for expressing a polypeptide in a cell (e.g., an expression vector), a Wobble-type substitution will result in the expression of a polypeptide with the same amino acid sequence as that originally encoded by the DNA molecule. As described above, however, substitutions may be conservative, or non-conservative, or any combination thereof.

A skilled artisan will be able to determine suitable variants of the polypeptides and/or fragments (e.g., peptides and/or proteins) provided herein using well-known techniques. For identifying suitable areas of the molecule that may be changed without destroying biological activity (e.g. immunogenicity, MHC binding, red blood cell (RBC) agglutination, RBC hemolysis), one skilled in the art may target areas not believed to be important for that activity. For example, when derivatives with similar activities from the same species or from other species are known, one skilled in the art may compare the amino acid sequence of a polypeptide to such similar polypeptides. By performing such analyses, one can identify residues and portions of the molecules that are conserved. It will be appreciated that changes in areas of the molecule that are not conserved relative to such similar derivatives would be less likely to adversely affect the biological activity and/or structure of a polypeptide. However, modifications resulting in decreased binding to MHC will not be appropriate in most situations. One skilled in the art would also know that, even in relatively conserved regions, one may substitute chemically similar amino acids for the naturally occurring residues while retaining the desired characteristics of the polypeptide and/or fragment. Therefore, even areas that may be important for biological activity or for structure may be subject to conservative amino acid substitutions without destroying the biological activity or without adversely affecting the structure of the derivative.

Analogs can differ from naturally occurring P. gingivalis polypeptides in amino acid sequence and/or by virtue of non-sequence modifications. Non-sequence modifications include changes in acetylation, methylation, phosphyorylation, carboxylation, or glycosylation. A “modification” of a polypeptide of the present invention includes polypeptides (or analogs thereof, such as, e.g. fragments thereof) that are chemically or enzymatically derivatized at one or more constituent amino acid. Such modifications can include, for example, side chain modifications, backbone modifications, and N- and C-terminal modifications such as, for example, acetylation, hydroxylation, methylation, amidation, and the attachment of carbohydrate or lipid moieties, cofactors, and the like, and combinations thereof. Modified polypeptides of the invention may retain the biological activity of the unmodified polypeptides or may exhibit a reduced or increased biological activity.

### Polypeptide Sequence Similarity and Polypeptide Sequence Identity

Structural similarity of two polypeptides can be determined by aligning the residues of the two polypeptides (for example, a candidate polypeptide and the polypeptide of, for example, any appropriate AP provided herein) to optimize the number of identical amino acids along the length of their sequences; gaps in either or both sequences are permitted in making the alignment in order to optimize the number of identical amino acids, although the amino acids in each sequence must nonetheless remain in their proper order. A candidate polypeptide is the polypeptide being compared to the reference polypeptide. A candidate polypeptide can be isolated, for example, from a microbe (e.g., P. gingivalis), or can be produced using a recombinant techniques, or chemically or enzymatically synthesized.

A pair-wise comparison analysis of amino acids sequences can be carried out using a global algorithm for example Needleman-Wunsch. Alternatively, polypeptides may be compared using a local alignment algorithm such as the Blastp program of the BLAST 2 search algorithm, as described by Tatiana et al., (FEMS Microbiol. Lett, 174:247-250 (1999), and available on the National Centre for Biotechnology Information (NCBI) website. The default values for all BLAST 2 search parameters may be used, including matrix=BLOSUM62; open gap penalty=11, extension gap penalty=1, gapxdropoff=50, expect 10, wordsize=3, and filter on. The Smith and Waterman algorithm is another local alignment tool that can be used (1988).

In comparison of two amino acid sequences, structural similarly may be referred to by percent “identity” or may be referred to by percent “similarity.” “Identity” refers to the presence of identical amino acids. Unless otherwise stated, the term “percent identity” means that a pair-wise comparison analysis of two amino acids was carried out using a global algorithm. “Similarity” refers to the presence of not only identical amino acids but also the presence of conservative substitutions. A conservative substitution for an amino acid in a polypeptide of the invention may be selected from other members of the class to which the amino acid belongs, as shown on Table 0.2.

An AP polypeptide as provided herein can include a polypeptide with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or least 99%, amino acid sequence identity to the reference amino acid sequence (e.g., a specified AP sequence).

### Fusions

In some embodiments, the polypeptides and/or fragments described herein may include fusion polypeptide segments that assist in purification or detection of the polypeptides. Fusions can be made either at the amino terminus or at the carboxy terminus of the subject polypeptide variant thereof. Fusions may be direct with no linker or adapter molecule or may be through a linker or adapter molecule. A linker or adapter molecule may be one or more amino acid residues, typically from about 20 to about 50 amino acid residues. A linker or adapter molecule may also be designed with a cleavage site for a DNA restriction endonuclease or for a protease to allow for the separation of the fused moieties. It will be appreciated that once constructed, the fusion polypeptides can be derived according to the methods described herein. Suitable fusion segments include, among others, metal binding domains (e.g., a poly histidine segment), immunoglobulin binding domains (i.e., Protein A, Protein G, T cell, B cell, Fc receptor, or complement protein antibody binding domains), sugar binding domains (e.g., a maltose binding domain), and/or a “tag” domain (i.e., at least a portion of galactosidase, a strep tag peptide, a T7 tag peptide, a FLAG peptide, or other domains that can be purified using compounds that bind to the domain, such as monoclonal antibodies). This tag is typically fused to the polypeptide upon expression of the polypeptide, and can serve as a means for affinity purification of the sequence of interest polypeptide from the host cell. Affinity purification can be accomplished, for example, by column chromatography using antibodies against the tag as an affinity matrix. Optionally, the tag can subsequently be removed from the purified sequence of interest polypeptide by various means such as using certain peptidases for cleavage. Examples of fusion proteins with a segment/domain attached at the N-terminus to aid in purification are provided herein (see e.g., SEQ ID NOs: 7 and 22).

In some embodiments, the AP comprises, consists, or consists essentially of a combination of a GST-TEV-gingipain-His as a fusion protein (e.g., SEQ ID Nos. 85-87), or a variant thereof that is at least 70, 75, 80, 85, 90, 95, 96, 97, 98, 99, or more percent identical or similar to SEQ ID Nos. 85-87 fused together. In some embodiments, this can be used in any of the compositions and/or methods provided herein.

In some embodiments, the AP comprises, consists, or consists essentially of a combination of GST -gingipain as a fusion protein (e.g., SEQ ID No: 90), or a variant thereof that is at least 70, 75, 80, 85, 90, 95, 96, 97, 98, 99, or more percent identical or similar to SEQ ID Nos. 90. In some embodiments, this can be used in any of the compositions and/or methods provided herein.

In some embodiments, the AP comprises, consists, or consists essentially of a combination of a KGP-RGP chimeric (e.g., SEQ ID Nos. 88-89), or a variant thereof that is at least 70, 75, 80, 85, 90, 95, 96, 97, 98, 99, or more percent identical or similar to SEQ ID Nos. 88-89 fused together. In some embodiments, this can be used in any of the compositions and/or methods provided herein.

In some embodiments, the AP comprises, consists, or consists essentially of a combination of a HagA 3× recombinant epitope (e.g., SEQ ID No: 84), or a variant thereof that is at least 70, 75, 80, 85, 90, 95, 96, 97, 98, 99, or more percent identical or similar to SEQ ID Nos. 84. In some embodiments, this can be used in any of the compositions and/or methods provided herein.

In certain embodiments, the polypeptides and/or fragments may be directly or indirectly (e.g., using an antibody) labeled or tagged in a manner which enables it to be detected. Labels include fluorochromes such as fluorescein, rhodamine, phycoerythrin, Europium and Texas Red, chromogenic dyes such as diaminobenzidine, radioisotopes, macromolecular colloidal particles or particulate material such as latex beads that are colored, magnetic or paramagnetic, binding agents such as biotin and digoxigenin, and biologically or chemically active agents that can directly or indirectly cause detectable signals to be visually observed, electronically detected or otherwise recorded, for example in a FACS, ELISA, Western blot, TRFIA, immunohistochemistry, evanescence, Luminex bead array, or dipstick or other lateral flow assay format. Suitable antibody-binding molecules for use in such methods may include immunoglobulin-binding antibodies, for example anti-human Ig antibodies, anti-human Ig antibodies, anti-human antibodies specific for Ig isotypes or for subclasses of IgG, or specific for P. gingivalis proteins.

Preferred fluorescent tag proteins include those derived from the jelly fish protein known as green fluorescent protein (GFP). Further information on GFP and other fluorophores is given in the following publications: Tsien R Y, “The Green Fluorescent Protein” Annual Reviews of Biochemistry 1998; 67:509-544 Verkhusha, V. and Lukyanov, K. “The Molecular Properties and Applications of Anthoza Fluorescent Proteins and Chromophores” Nature Biotechnology 2004; 22:289-296. Plasmid vectors encoding a wide range of fluorescent tag proteins are commercially available from various suppliers including an array of “Living Colours&#8482; Fluorescent Proteins” available commercially from Clontech Laboratories, Inc. Similar vectors can also be obtained from other suppliers including Invitrogen and Amersham Biosciences. Suitable fluorescent proteins derived from GFP are the red-shifted variant EGFP, the cyan shifted variant ECFP and the yellow shifted variant EYFP. EGFP is preferred as the fluorescent marker because it gives bright fluorescence combined with minimal effect on the antigenic properties of the target antigen. Alternative fluorescent marker proteins are commercially available. Biologically or chemically active agents include enzymes, which catalyse reactions that develop or change colors or cause changes in electrical properties, for example, and may also be utilized. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed. Further examples include horseradish peroxidase and chemiluminescence. In some embodiments, the non-immobilized antibody-binding molecule or polypeptide may be detected using an antibody which binds to said non-immobilized antibody-binding molecule or polypeptide. A suitable detection antibody may be labeled by means of fluorescence. The label may be a fluorescent marker (tag) which is used to label the target antigen directly such that the antigen and the fluorescent marker form a fusion protein.

### Methods

Also provided herein are methods of using an immunogenic composition (such as an AP) that serves as a vaccine for Porphyromonas gingivalis, as described herein, to treat a subject in need of treatment, e.g., for periodontal disease and/or acute/chronic systemic and organ inflammation. In some embodiments, the condition, disorder or disease is, without limitation, one or more of vascular disease (e.g., cardiovascular disease, atherosclerosis, coronary artery disease, myocardial infarction, stroke, and cardiac hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD age related macro-degeneration, cerebral and abdominal aneurysms, glioma, large vessel stroke C-IMT, microvascular defects and associated dementias (e.g., Parkinson's), Peri-Implantitis and/or periodontal disease and/or associated bone loss, cognitive disorders (e.g., early, middle, and/or late dementia; Alzheimer's disease); regenerative and stem cell dysfunction; and age-related disorder.

While all APs provided herein are contemplated for use in the methods provided herein (unless otherwise specified), in some especially advantageous embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In general terms, the method includes administering a therapeutically effective amount of an AP that allows for vaccination, as described herein, to a subject having an active and/or subclinical infection with or without periodontal disease or inflammation, e.g., gingivitis or periodontitis. In some embodiments, the method includes administering to the subject a therapeutically effective amount of an AP, as described herein, to a subject having P. gingivalis localized in the sub-gingival gum line, either with or without gingivitis, and/or periodontal disease or inflammation. In some embodiments, the AP for use in the present methods assists in the outer membrane forming vesicles and/or secreted outer membrane vesicles containing arg and Lys gingipains/adhesins/hemagglutinins/LPS. In some embodiments, the method includes administering to the subject a therapeutically effective amount of an AP to a subject having P. gingivalis localized in the sub-gingival gum line and leaking or trans-migrating through epithelia cells and into local lymphatic drainage and the blood vascular system. In some embodiments, the method is a method for immunization of a subject against a periodontal infection (such as gingivitis or periodontitis) by administering the AP, as described herein. In some embodiments, the method is a method for active, topical oral active administration of a subject against a periodontal infection (such as gingivitis or periodontitis) by administering the AP, as described herein.

The AP can be administered using any suitable route to treat the infection, e.g., periodontal infection. In some embodiments, the AP is administered orally, subgingivally, subcutaneously, intradermally, or intravenously. In some embodiments, the infection is an infection of the gingiva (e.g. gingivitis or periodontitis), blood vessels, the lungs, heart, liver gastro-intestinal tract, brain, etc., and the method includes subgingivally placing a therapeutically effective amount of the AP into the subject. The AP may be placed subgingivally in any suitable manner to treat the periodontal infection. In several embodiments, the AP is placed subgingivally at 1, 2, 3, 4, 5, or 6 or more sites around each tooth to be treated. In some embodiments, the AP is placed subgingivally at or around each tooth in a subject's mouth. In some embodiments, the AP is placed subgingivally at or around each of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32 teeth in a subject's mouth. In some embodiments, the AP is placed subgingivally at or around one or more of the subject's incisor, canine, premolar and/or molar tooth. In some embodiments, the AP is administered at about 0.001, 0.005, 0.01, 0.02, 0.05, 0.1, 0.2, 0.5, 1, 1.2, 1.5, 2, 2.2, 2.5, 3, 3.2, 3.5, 4, 4.2, 4.5, 5, 5.2, 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 70, 80, 90, or 100 μg of the AP per tooth, or an amount in between any two of the preceding values. In some embodiments, the AP is administered at about 0.5-10 μg, about 1-8 μg, about 1.5-6 μg, or about 2-5 μg of the AP per tooth in a treatment. In some embodiments, the AP is administered at about 3 μg per tooth in a treatment. In some embodiments, the I AP is administered at about 10-400 μg, about 30-300 μg, about 50-200 μg, about 60-160 μg of the AP per a subject's mouth in a treatment. In some embodiments, the AP is administered at about 96 μg per subject's mouth in a treatment.

In some embodiments, the method includes removing a microbial infection or preventing its re-colonization in a supra- and/or subgingival space of the subject, before administering the AP. In certain embodiments, the method includes removing plaque from the supra- and/or subgingival space of the subject, before administering the AP. In some embodiments, the AP is placed subgingivally after removing plaque from the supra- and/or subgingival space of one or more teeth to be treated. Plaque can be removed using any suitable means. In some embodiments, the plaque is removed by cleaning and/or root planning. In some embodiments, the method includes administering one or more antibiotics to the subject to remove a microbial infection or colonization in a supra- and/or subgingival space of the subject.

In some embodiments, administration of the AP prevents or prolongs the time before recolonization. “Recolonization” as used herein refers to detectable growth of P. gingivalis in a supra- and/or subgingival plaque after initial removal of P. gingivalis.

In some embodiments, methods of the present disclosure reduces or eliminates a P. gingivalis infection in the subject, e.g., in the subgingival space of the subject. In some embodiments, the P. gingivalis infection is reduced on average about 10% or more, e.g., 20% or more, 30% or more, 40% or more, 50% or more, 60% or more, 70% or more, 80% or more, 90% or more, 95% or more, including about 100%, compared to the pretreatment level of infection.

In some embodiments, methods of the present disclosure prevent recolonization and or initial colonization of the gingiva by P. gingivalis. Recolonization is inhibited when P. gingivalis growth is inhibited after initial removal of P. gingivalis from the gingival and/or subgingival space, e.g., by removal of plaque. Thus, the method in some embodiments includes removing P. gingivalis from a subgingival space of the subject before administering the AP to the subject. In some embodiments, removing P. gingivalis from a subgingival space includes cleaning and/or root planning to thereby remove plaque from the subgingival space.

In some embodiments, recolonization is inhibited when P. gingivalis remains undetectable, or detectable at 5% or less, 3% or less, 2% or less, or 1% or less, in a subgingival plaque sample, after initial removal of P. gingivalis from the gingival and/or subgingival space. In some embodiments, recolonization is inhibited for about 1, 2, 3, 4, 5, 6, 7, 8, 9, 10, 11, 12, 13, 14, 15, 16, 17, 18, 19, 20, 21, 22, 23, 24 months or more, or for any period of time in between any two of the times listed above, after initial removal of P. gingivalis. P. gingivalis may be detected by, e.g., immunofluorescent staining of a plaque sample using KB-001.

The AP can be administered according to any suitable dosing regimen, depending on the embodiment. The dosing regimen may depend on, for example, the severity of periodontal disease (e.g., gingivitis or periodontitis), and/or the strain of P. gingivalis involved in the periodontal disease (e.g., the virulence of the strain, the amino acid sequence of the AP target expressed by the strain, etc.). In some embodiments, an effective dose of the AP can be administered once to a subject. In some embodiments, an effective dose of the AP can be administered repeatedly to a subject, e.g., at least 2, 3, 4, 5, 6, 7, 8, 9, 10, 12, 15, 20, 25, 30, 40 or 50 times or more, or any number of times in between any two of the numbers listed above. In some embodiments, the method includes administering the AP at an interval of about 1, 2, 3, 4, 5, 6, 7, 8, 9, 10, 11, 12, 13, 14, 15, 16, 17, 18, 19, 20, 25, 30, 40, or about 50 days between any two consecutive doses. In some embodiments, the method includes administering the AP 1-5 days, 6-10 days, 10-16 days, 16-20 days, 20-25 days, 25-30 days, 30-35 days, 35-40 days, including 40-50 days between any two consecutive doses. In some embodiments, after an initial dosing regimen, the AP can be administered on a less frequent basis. For example, after weekly or biweekly administration for three months, treatment can be repeated once per month, for six months or a year or longer.

For systemic administration, subjects can be administered a therapeutic amount of the AP, such as, microgram to milligram, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

The dosage of an AP as described herein can be determined by a physician and adjusted, as necessary, to suit observed effects of the treatment. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. In some embodiments, the dosage is 2-3 doses over a 1-2 month period sometime 2-3 months sometimes 3-4 months.

The dosage ranges for the administration of the AP described herein, according to the methods described herein depend upon, for example, the form of the AP, its potency, and the desired outcome, e.g., the extent to which symptoms are to be reduced, level of markers, or other indicators of a condition, such as inhibition of recolonization. The dosage should not be so large as to cause adverse side effects. The dosage can vary with the age, condition, and sex of the patient and can be determined by one of skill in the art.

Administering the AP may be done using any suitable option. In some embodiments, the AP is administered using a syringe, e.g., a Hamilton syringe. In some embodiments, the AP is administered using a syringe equipped with a suitable gauge needle. In some embodiments, the AP is administered with a blunt small gauge needle attached to the syringe. In some embodiments, a gene delivery system, such as a gene transdermal patch can be used.

Any suitable delivery system for intraoral, interproximal, intrasulcular, intraperiodontal pocket, intracanal, and intranasal delivery of the AP can be used to administer the AP to an oral site. Suitable systems can be, without limitation, mechanical or automated, dental or medical syringes, calibrated or non-calibrated. In some embodiments, a delivery system includes one or more attachments. The delivery system can have any suitable tip, including, but not limited to, blunt ended, and side port. In some embodiments, the delivery system includes a medicament delivery tray and systems, including, without limitation, PerioProtect Trays. In some embodiments, the delivery system includes a medicament applicator delivery system. In some embodiments, the delivery system includes a slow releasing medical preparation, e.g., for intrasulcular drug delivery. In some embodiments, a delivery system includes, without limitation, a filler, oral packing, fiber, microparticles, films, gels, injectable gels, vesicular systems, strips compacts, chip, hydrogel, thermal gel, liquid, solid, including, but not limited to, Actisite, Arestin, Atridox, Ossix Plus, Periochip, Periostat, Periofil. In some embodiments, the delivery system is an injectable system. In some embodiments, the delivery system is an irrigation system including, but not limited to piezoelectric or ultrasonic cavitron units, with or without reservoir, including, without limitation, Ora-Tec Viajet and Oral irrigation systems, including, without limitation, Interplak, Waterpik, Hydrofloss, Viajet, Airfloss and Pro.

In some embodiments, a subject has been diagnosed with a condition or disease, e.g., a P. gingivalis infection, chronic inflammation, Alzheimer's disease, etc., that may be treated with a method of the present disclosure. In some embodiments, the subject is diagnosed with a condition or disease using a kit for detecting the presence of P. gingivalis on the subject, e.g., at a site of infection. In some embodiments, the kit is configured to detect the presence of P. gingivalis in a gingival environment of the subject. In some embodiments, the kit includes instructions for using the kit and/or provide the subject with recommendations to seek treatment based on the result of the diagnosis.

### Various Applications

Without being bound to theory, P. gingivalis is thought to relocate into various other tissues/organs/end capillary beds throughout the body and cause local inflammation at these sites. In some embodiments, delivering an AP of the present disclosure addresses the systemic infection or distant infections at one or more secondary sites. In some embodiments, an AP allows for deeper tissue penetration, e.g., to treat various P. gingivalis related cancers.

A variety of conditions, disorders or diseases may be treated through the use of an AP of the present disclosure. Without being limited by theory, the use of the AP of the present disclosure to eliminate and/or prevent re-colonization of P. gingivalis in the sub-gingival gum line can in some embodiments interrupt and/or block, or over express the host's inflammatory pathways, such as the inflammasome NLRP3/Interleukin-1β/IL-6 pathways, AIM2, C-reactive protein, the PCSK9 pathway, and the Interleukin-1β innate immunity pathway. In addition, the local and systemic secretion by the bacteria of tissue-damaging outer-membrane vesicles containing a potent mixture of toxins can be curtailed. The AP of the present disclosure can, in certain embodiments, allow for specifically and locally targeting the P. gingivalis oral infection, which can be the root cause of a chronic active inflammation and toxemia throughout the host's body. In some embodiments, use of the AP to specifically target and eliminate the disease-causing bacterial source, while sparing other existing oral bacterial strains, provides for treatment of the systemic inflammation without interrupting the complex host inflammation pathways. In some embodiments, use of AP as disclosed herein avoids or reduces local and/or systemic side effects that may result from intervening in the disrupting/reducing/overexpressing inflammatory pathways such as but not limited to inflammasome NLRP3/Interleukin-1β/IL-6 pathways, C-reactive protein, the PCSK9 pathway, and the Interleukin-1β innate immunity pathway for treating a disease.

In some embodiments, a P. gingivalis infection at an oral site affects end organs, such as, without limitation, large and small vessels of the heart, carotid arteries, vessels in the brain, liver, joints, lungs, pancreas, reproductive system. In some embodiments, the condition, disorder or disease is, without limitation, one or more of vascular disease (e.g., cardiovascular disease, atherosclerosis, coronary artery disease, myocardial infarction, stroke, and cardiac hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD age related macro-degeneration, cerebral and abdominal aneurysms, glioma, large vessel stroke C-IMT, microvascular defects and associated dementias (e.g., Parkinson's), Peri-Implantitis, periodontal disease and/or associated bone loss, cognitive disorders (e.g., early middle late dementia Alzheimer's disease); regenerative and stem cell dysfunction; and age-related disorder. In some embodiments, the method involves any one of the above disorders, where the disorder is caused or complicated by P. gingivalis.

Conditions, disorders or diseases treated by administration of an AP of the present disclosure includes, without limitation, vascular disease (e.g., cardiovascular disease, atherosclerosis, coronary artery disease, myocardial infarction, stroke, and cardiac hypertrophy); systemic disease (e.g., type II diabetes, insulin resistance and metabolic syndrome); rheumatoid arthritis; cancer (e.g., oral squamous carcinomas, gastrointestinal cancer, pancreatic cancer, lung cancer, etc); gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); cognitive disorder (e.g., Alzheimer's disease); and longevity and/or age-related disorders. In general terms, the method includes identifying a subject in need of treating a condition, disorder or disease, as disclosed herein, and administering to the subject a therapeutically effective amount of the AP of the present disclosure, to thereby treat the condition, disorder or disease.

In some embodiments, the condition, disorder or disease is a vascular disease. A variety of vascular diseases can be treated by use of the present APs. In some embodiments, the vascular disease is, without limitation, cardiovascular disease, atherosclerosis, coronary artery disease, myocardial infarction, stroke, or cardiac hypertrophy. Without being bound by theory, P. gingivalis and its virulence factors (e.g., outer membrane vesicles (OMVs), LPS, peptidylarginine deiminase (PPAD), gingipains, hemagglutinins, and fimbriae) are thought to disrupt the inflammatory pathways of heart and systemic vascular disease (CVD/Stroke), including the NLRP3/Interleukin-1β/IL-6 pathways, C-reactive protein (CRP) elevation, the PCSK9 pathway, and the suppression of adaptive immunity via reduction of regulatory T cells (Tregs). P. gingivalis infection can be associated with an increased risk of heart attack, and P. gingivalis is involved with forming oxidized LDL taken up by macrophages, leading to foam cell formation. These atherosclerotic lesions can develop a necrotic core, often forming a thrombus, leading to a downstream event (i.e. heart attack, stroke). Periodontal disease and/or P. gingivalis can be associated with elevated levels of systemic inflammatory markers, such as CRP, IL-6, and Lp-PLA2, Hb-A1c, IL-1b. P. gingivalis can play a major role in Abdominal Aortic Aneurysm development and salivary MPO enzyme activity. Periodontal therapy, as an intervention for improved oral health, can facilitate the management of thrombotic risk, and in the long term can contribute to the prevention of cardiovascular events in patients at risk.

In some cases, the development of atherosclerosis is due to systemic inflammation caused by severe periodontitis. Without being bound by theory, systemic inflammation induced by severe periodontitis, such as those associated with enhanced the secretion of pro-inflammatory cytokines from macrophages and increased the adhesion of monocytes to endothelial cells induce by P. gingivalis LPS, can exacerbate atherosclerosis via, in part, causing aberrant functions of vascular endothelial cells and the activation of macrophages. Further, patients with periodontitis can show higher serum pro-inflammatory cytokines such as tumor necrosis factor (TNF)-α, interleukin (IL)-1β, or IL-6. P. gingivalis can alter genes responsible for mitochondrial function and downregulate gene expression in the signaling pathway, which can lead to mitochondrial dysfunction and metabolic imbalance that promote the development of atherosclerosis. In some embodiments, P. gingivalis can prevent the regression of atherosclerotic plaques by interfering with reverse cholesterol transport. P. gingivalis can also promote atherosclerosis through alteration of gut microbiota, increased IL-1β, IL-18, and TNF-α production in peritoneal macrophages and gingival or aortic gene expression of the NOD-like receptor family, NLRP3, IL-1β, pro-IL-18 and pro-caspase-1, activation of the NLRP3 inflammasome, e.g., through CD36/SR-B2 and TLR2.

Chronic periodontitis (CP) can be associated with increased serum levels of HDL, Ox-LDL, hs-CRP, Hb-A1c, Lp-PLA2, MPO, LDH, troponins T & I, NT pro-BNP, and P selectin. Further, infection of type II P. gingivalis can cause prolonged cytokine response such as IL-1β, IL-8 and TNFα. Elevated cardiac markers found in periodontitis patients indicates that they may carry potential risks in developing cardiac lesions.

In some cases, P. gingivalis contribute to endothelial dysfunction and/or atherosclerotic cardiovascular disease. Without being limited by theory, P. gingivalis may cause vascular damage and increased endothelial permeability by degrading, via gingipain proteases, platelet endothelial cell adhesion molecule-1, and vascular endothelial cadherin, which play a role in endothelial junctional integrity. The vascular damage can increase endothelial permeability and initiate several processes implicated in atherosclerosis, including platelet aggregation, induction of proinflammatory cytokine release, and promotion of leukocyte extravasation to subendothelial regions.

Further, P. gingivalis promotes cardiac rupture after myocardial infarction (MI). Without being bound by theory, P. gingivalis is thought to invade the ischemic myocardium, promote cardiomyocyte apoptosis through activation of p18 Bax by gingipain, increase oxidative stress and MMP-9 protein level and activity, causing cardiac rupture. P. gingivalis-secreted factors can also promote cardiac hypertrophy, through activation of MEK/ERK signal pathways, Toll-like receptor-2 signaling. In some cases, mitogen-activated protein kinase kinase is involved in P. gingivalis-induced myocardial cell hypertrophy and apoptosis. In some cases, components of P. gingivalis spent culture medium increases total MEK-1 and ERK-1 protein products, but also causes increased cellular size, DNA fragmentation, and nuclear condensation in H9c2 cells. These three parameters, and the phosphorylated ERK-1 protein products of H9c2 cells treated with P. gingivalis medium, can be significantly reduced after pre-administration of U0126. The results indicate that P. gingivalis-secreted factors may initiate MEK/ERK signal pathways and lead to myocardial cell hypertrophy and apoptosis.

In some cases, P. gingivalis induces myocardial hypertrophy through Toll-like receptor-2 signaling in the isoproterenol-induced myocardial hypertrophy model. Regulation of chronic inflammation induced by periodontitis may have a key role in the treatment of myocardial hypertrophy. In some embodiments, P. gingivalis enhances myocardial vulnerability, thereby promoting post-infarct cardiac rupture. In some embodiments, Infection with Porphyromonas gingivalis (P.g.) promotes cardiac rupture after MI; P.g. invades the ischemic myocardium; Infection with P.g. promotes the accumulation of p18 Bax; Gingipains from P.g. activate Bax and promote cardiomyocyte apoptosis; Infection with P.g. promotes oxidative stress and MMP-9 protein level and activity.

In some situations, infection with periodontal pathogens can cause an adverse outcome after myocardial infarction (MI). This has been shown by a variety of studies, including one where C57BL/6J mice were inoculated with Porphyromonas gingivalis (P.g.), a major periodontal pathogen, or injected with phosphate-buffered saline (PBS) into a subcutaneously-implanted steelcoil chamber before and after coronary artery ligation. A significant increase in mortality, due to cardiac rupture, was observed in the P.g.-inoculated MI mice. Ultrastructural examinations revealed that P.g. invaded the ischemic myocardium of the P.g.-inoculated MI mice. The expression of p18 Bax, an active form of pro-apoptotic Bax protein, markedly increased in the P.g.-inoculated MI hearts. In vitro experiments demonstrated that gingipain, a protease uniquely secreted from P.g., cleaved wild type Bax at Arg34, as evidenced by the observation that the cleavage of Bax by gingipain was completely abolished by the Arg34Ala mutation in Bax. Treatment with immunoglobulin Y against gingipain significantly decreased the mortality of the P.g.-inoculated MI mice caused by cardiac rupture. Furthermore, inoculation of P.g. also resulted in an increase of MMP-9 activity in the post-MI myocardium by enhancing oxidative stress, possibly through impairing the selective autophagy-mediated clearance of damaged mitochondria. Without being bound by theory, infection with P.g. during MI can play a detrimental role in the healing process of the infarcted myocardium by invasion of P.g. into the myocardium, thereby promoting apoptosis and the MMP-9 activity of the myocardium, which, in turn, can cause cardiac rupture.

In some cases, P. gingivalis induces cellular hypertrophy and MMP-9 activity via different signaling pathways in H9c2 cardiomyoblast cells. P. gingivalis medium can elevate MMP-9 activity and induce cardiomyoblast hypertrophy. P. gingivalis-induced H9c2 cell hypertrophy was mediated through p38, ERK, PI3K, calcineurin, and JNK signaling pathways, which are in a totally different regulatory pathway from P. gingivalis-elevated MMP-9 activity. P. gingivalis infection activated multiple factors via different pathways to induce the development of hypertrophy of H9c2 cardiomyoblast cells.

In some cases, P. gingivalis deteriorates Isoproterenol-Induced myocardial remodeling in mice. In some situations, stronger cardiomyocyte hypertrophy can be observed in the ISO(+)/P.g.(+) mice compared with the ISO(+)/P.g.(−) mice. The total square of randomly selected cardiomyocytes was 23% larger in the ISO(+)/P.g.(+) mice than in the ISO(+)/P.g.(−) mice. A higher level of mRNA expression in Toll-like receptor 2 and NADPH oxidase 4 in the ISO(+)/P.g.(−) mice was detected compared with the control group. A periodontal pathogen affected ISO-induced cardiac hypertrophy via oxidative stress.

In some situations, P. gingivalis-related cardiac cell apoptosis can be co-activated by p38 and extracellular signal-regulated kinase pathways. In some situations, the development of cardiac cell apoptosis can be directly induced by P. gingivalis medium. Porphyromonas gingivalis-related H9c2 cell apoptosis was mainly co-activated by p38 and ERK pathways and may be involved in death receptor-dependent (caspase 8) and mitochondria (caspase 9)-dependent apoptotic pathways. Porphyromonas gingivalis-related cardiac cell apoptosis was also partially mediated by PI3K or calcineurin signaling pathways, whereas the JNK pathway might play a protective role in P. gingivalis-related cardiac cell apoptosis.

In some situations, the miRNA-212/132 family regulates both cardiac hypertrophy and cardiomyocyte autophagy. In some situations, miR-212/132 family has a key role in cardiac hypertrophy and heart failure development. Both miR-212 and miR-132 can target and negatively regulate the expression of the FoxO3 transcription factor, a powerful anti-hypertrophic and pro-autophagic factor in cardiomyocytes. The microRNA (miRNA)-212/132 family can regulate cardiac hypertrophy and autophagy in cardiomyocytes.

In some situations, Porphyromonas gingivalis-induced miR-132 regulates TNFα expression in THP-1 derived macrophages Live P. gingivalis infection induced miR-132 via TLR signaling and activation of NF-KB. Furthermore, inhibition of miR-132 expression strongly repressed the production of TNFα and increased NFE2L2 and NFAT5. Without being bound by theory, miR-132 modulates TNFα via inhibition of its target genes, which is believed to provide a path for intervention for P. gingivalis-induced TNFα associated diseases such as periodontitis.

Thus, APs of the present disclosure targeting P. gingivalis can be used to address these (any of the above or following) disorders, conditions or diseases, in some embodiments. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some embodiments, the condition, disorder or disease treated by the present methods is a wound. In some embodiments, administration of an AP of the present disclosure promotes wound closure and/or prevents or reduces P. gingivalis-induced inhibition of wound closure. In some embodiments, a novel gingipain regulatory gene in Porphyromonas gingivalis mediates host cell detachment and inhibition of wound closure.

In some situations, the pgn_0361 gene is involved in regulating gingipains. The PGN_0361-defective strain of P. gingivalis exhibited reduced virulence in terms of epithelial cell detachment and inhibition of wound closure. The culture supernatant of the mutant strain can highly inhibit wound closure, which may be due to high gingipain activity.

In some situations, the capsular polysaccharide and the Arg- and Lys-gingipains of P. gingivalis influences the capacity of P. gingivalis to hinder wound healing, while LPS and the major fimbriae may have no effect. In some situations, entry of Porphyromonas gingivalis Outer Membrane Vesicles into Epithelial Cells Causes Cellular Functional Impairment. Without being bound to theory, loss of intracellular TfR due to MVs causes serious impairment of cellular migration and proliferation. Fundamental cellular operations, including DNA synthesis and ATP generation, require iron, while transferrin-TfR complexes are internalized and ferric iron is released from transferrin at endosomal pH levels. TfR degradation by P. gingivalis can cause impairment of cellular functions, and it is notable that TfR is a target molecule of the bacterium. Thus, APs of the present disclosure targeting P. gingivalis can be used to address these disorders, conditions or diseases in some embodiments. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some embodiments a balanced oral pathogenic bacteria and probiotics can promote wound healing via maintaining mesenchymal stem cell homeostasis. In some cases, P. gingivalis inhibits the functions of mesenchymal stem cells (MSCs) by activating NLRP3 inflammasome. LPS increase in P. gingivalis and thereby inhibits the functions of MSCs by activating NLRP3 inflammasome. Without being bound by theory, homeostasis of oral microbiomes can play a role in maintaining oral heath, provide options for the prevention and treatment of oral diseases, and have referential value for other systemic diseases caused by dysfunction of microbiota and MSCs. Infection of hDFSCs with P. gingivalis can prolong the survival of neutrophils and increase their migration. These phenotypic changes can depend on direct cellular contacts and PPAD expression by P. gingivalis. Active JNK and ERK pathways in primed human dental follicle stem cells (hDFSCs) can be implicated in the phenotypic changes in neutrophils. In some cases, P. gingivalis can modify hDFSCs, thereby cause an immune imbalance and thus stem cell therapies may be improved and enhanced and protected by eliminating P.g. Thus, APs of the present disclosure targeting P. gingivalis can be used to address these disorders, conditions or diseases in some embodiments. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some embodiments, the condition, disorder or disease is age-related macular degeneration (AMD). In some situations, P. gingivalis invades human retinal pigment epithelial cells, leading to vacuolar/cytosolic localization and autophagy dysfunction. In some situations, Periodontal disease(PD) is linked to age-related macular degeneration (AMD). Porphyromonas gingivalis(Pg), a keystone oral-pathobiont, can be causative of PD, and can efficiently invades human gingival epithelial and blood-dendritic cells. Live, but not heat-killed Pg-strains can adhere to and invade ARPEs. This involves early adhesion to ARPE cell membrane, internalization and localization of Pg within single-membrane vacuoles or cytosol, with some nuclear localization apparent. No degradation of Pg or localization inside double-membrane autophagosomes was evident, with dividing Pg suggesting a metabolically active state during invasion. Significant downregulation of autophagy-related genes particularly, autophagosome complex, can be observed. Antibiotic protection-based recovery assay further can confirm distinct processes of adhesion, invasion and amplification of Pg within ARPE cells. P. gingivalis can invade human-RPEs, begin to characterize intracellular localization and survive within these cells. The dysbiotic periodontal pathogen P. gingivalis can efficiently invade retinal epithelial cells in high levels, replicate and are sustained within them. This invasion and autophagy evasion by the keystone species may be one of the contributing elements in the pathogenesis of retinal degenerative diseases. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some cases, invasion of RPE by Pg and mutants can elevate AMD-related genes involved in angiogenesis; immunosuppression and complement activation which might be the target molecules for both diseases. In some situations, infection of Porphyromonas gingivalis, A Keystone Bacterium in Periodontal Microbiota, is associated with a risk for diabetic retinopathy. In some situations, there is a significant association between a specific microbe in periodontal microbiota and DR, and oral microbiota play a role in retinal eye health.

In some situations, retinal blood flow and neurovascular are coupled in patients with Alzheimer's disease and mild cognitive impairment. In some situations, patients with MCI and AD, retinal blood flow and arterial vessel diameters can be reduced compared to healthy age- and sex-matched controls. No difference was found in flicker response between groups. This indicates alterations in retinal blood flow in patients with neurodegenerative disease. Thus, APs of the present disclosure targeting P. gingivalis can be used to address these disorders, conditions or diseases in some embodiments. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some embodiments, the condition, disorder or disease is autism. In some situations, Autism spectrum disorder (ASD) is associated with several oropharyngeal abnormalities, including dysbiosis in the oral microbiota. Since the oral cavity is the start of the gastrointestinal tract, this strengthens and extends the notion of a microbial gut-brain axis in ASD and even raises the question whether a microbial oral-brain axis exists. It is clear that oral bacteria can find their way to the brain through a number of pathways following routine dental procedures. A connection between the oral microbiota and a number of other brain disorders has been reported. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some situations, C1q as a regulator of brain development is implicated in autism spectrum disorders. Autism spectrum disorders (ASDs) represents a heterogeneous group of neurodevelopmental disorders with similar core features of social and communication impairments, restricted interests and repetitive behaviors. Early synaptic dysfunction due to neuroinflammatory insults may underpin the pathogenesis of abnormal brain development in some of individuals with ASDs. As a component of the innate immune response, the complement system can comprise both directly acting factors and factors that augment other components of the immune system. Beyond its involvement with innate immune responses in the brain, the complement system also plays important roles in neurodevelopment. Recent studies indicate involvement of complement component C1q in fundamental neurodevelopmental pathways and in maintenance and elimination of dendrites and synapses. The impact of aberrant complement system activity during critical windows of brain development may not only affect the local immune response but lead to atypical brain development. Thus, APs of the present disclosure targeting P. gingivalis can be used to address these disorders, conditions or diseases in some embodiments. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some embodiments, the condition, disorder or disease is large vessel stroke, C-IMT (Carotid Intima-media Thickness). In some cases, periodontal treatment can have an effect on carotid intima-media thickness in patients with lifestyle-related diseases. At baseline, LDL-C (low-density lipoprotein cholesterol) levels and percentage (%) of mobile teeth can be positively related to plasma IgG (immunoglobulin) antibody titer against P. gingivalis. Corresponding to improvements in periodontal clinical parameters after treatment, right and left max IMT (maximum intima-media thickness) levels cam be decreased significantly after treatment (SPT-S: start of supportive periodontal therapy, SPT-1y: at 1 year under SPT, and SPT-3y: at 3 years under SPT). P. gingivalis infection can be positively associated with progression of atherosclerosis. Without being bound by theory, routine screening using plasma IgG antibody titer against P. gingivalis and periodontal treatment under collaborative with medical and dental care may prevent cardiovascular accidents caused by atherosclerosis. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

P. gingivalis infection can be associated with LDL-C level, which facilitates atherosclerosis, and that periodontal treatment, in collaboration with medical care for atherosclerosis, may contribute to improvements in max carotid IMT. Plasma P. gingivalis IgG titer may be useful for the early detection of atherosclerosis. Finally, periodontal treatment is considered to be important for preventing the onset of cerebral and myocardial infarctions caused by atherosclerosis. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some situations, overall periodontal bacterial burden can be related to carotid IMT. In some situations, changes in clinical and microbiological periodontal profiles relate to progression of carotid intima-media thickness. In some situations, improvement in periodontal status—defined both clinically and microbiologically—is associated with less progression in carotid atherosclerosis in a randomly selected population-based sample of men and women. Accelerated atherosclerotic progression can be a mechanistic explanation linking periodontal disease and clinical CVD. Thus, APs of the present disclosure targeting P. gingivalis can be used to address these disorders, conditions or diseases in some embodiments. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some embodiments, the condition, disorder or disease is a systemic disease, e.g., a systemic metabolic disorder. A variety of systemic diseases can be treated by use of the present APs, as disclosed herein. In some embodiments, the systemic disease is, without limitation, type II diabetes, insulin resistance or metabolic syndrome. Without being bound by theory, P. gingivalis virulence factors can allow the pathogen's invasion to the periodontal tissue and subsequent dissemination into the systemic circulation, increasing the risk of systemic chronic diseases such as type 2 diabetes mellitus (T2DM), cardiovascular diseases, nonalcoholic fatty liver disease (NAFLD), rheumatoid arthritis, and Alzheimer disease. As used herein, “insulin resistance” refers to the reduction or loss of the response of the target organs and tissues to the biological effects of insulin, resulting in decreased efficiency of cell uptake and utilization of glucose and the occurrence of abnormal metabolism of glucose and lipids in cells. In some cases, P. gingivalis outer membrane vesicles (OMVs) can deliver gingipains to the liver, where gingipains can regulate hepatic glycogen synthesis by attenuating insulin sensitivity through the Akt/GSK-3β signaling pathway. Thus, P. gingivalis in the oral cavity can influence hepatic glucose metabolism by decreasing insulin sensitivity in the liver cells. Futher, P. gingivalis can induce insulin resistance through branched-chain amino acids (BCAA) biosynthesis. In addition, P. gingivalis/gingipain can translocate from the oral cavity to pancreatic islets and become localized primarily in β-cells, and may be epigenetically influencing development of bihormonal cells. Thus, APs of the present disclosure targeting P. gingivalis can be used to address these disorders, conditions or diseases in some embodiments. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some embodiments, the condition, disorder or disease is rheumatoid arthritis (RA). Without being bound by theory, antibodies against P. gingivalis have been found to be associated with RA and with anti-citrullinated protein antibodies (ACPA). Moreover, the DNA of P. gingivalis has been detected in the synovial fluid and plasma samples from patients with RA, and the coexistence of RA and periodontitis increased the probability of finding P. gingivalis DNA in these compartments. Clinical signs and symptoms of RA can improve after periodontal treatments and resolution of periodontitis. Thus, APs of the present disclosure targeting P. gingivalis can be used to address these disorders, conditions or diseases in some embodiments. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some embodiments, the condition, disorder or disease is cancer. In some embodiments, the cancer is, without limitation, oral, gastrointestinal, or pancreatic cancer. In some embodiments, the cancer is, without limitation, esophageal squamous cell carcinoma, head and neck (larynx, throat, lip, mouth and salivary glands) carcinoma. Without being bound to theory, P. gingivalis can promote distant metastasis and chemoresistance to anti-cancer agents and accelerate proliferation of oral tumor cells by affecting gene expression of defensins, by peptidyl-arginine deiminase and noncanonical activation of β-catenin. In some cases, the pathogen can convert ethanol to the carcinogenic intermediate acetaldehyde. In addition, P. gingivalis can be implicated in precancerous gastric and colon lesions, esophageal squamous cell carcinoma, head and neck (larynx, throat, lip, mouth and salivary glands) carcinoma, and pancreatic cancer. P. gingivalis can have systemic tumorigenic effects in addition to the local effects in its native territory, the oral cavity. Thus, APs of the present disclosure targeting P. gingivalis can be used to address these disorders, conditions or diseases in some embodiments. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some embodiments, an AP of the present disclosure may be administered in conjunction with one or more cancer therapy agents, e.g., chemotherapeutic agent, to enhance the therapeutic effect of the cancer therapy agent. In some embodiments, the cancer therapy agent is a small molecule drug, or an immunotherapeutic agent. In some cases P. gingivalis, its OMVs and/or gingipains have been found to cause an overall immunosuppression of the host, suppressing the adaptive immune system and altering the innate immune system. Adjuvant therapy of eliminating P.g. for improved outcomes for current and future chemotherapies. In some cases, P. gingivalis can inhibit drug induced apoptosis as well as necrosis (at least the LDH release) in the esophageal squamous cell carcinoma cell line EC0706. When the cancer cells are infected with P. gingivalis prior to the treatment with cisplatin, both apoptosis and necrosis is significantly reduced. Tumor xenografts composed of P. gingivalis-infected OSCC cells can exhibit a higher resistance to Taxol through Notch1 activation, as compared with uninfected cells. Furthermore, P. gingivalis-infected OSCC cells can form more metastatic foci in the lung than uninfected cells. Sustained infection with P. gingivalis, can promote distant metastasis of oral cancer, as well as its resistance to anti-cancer agents. Oral cancer cells sustainedly infected with Porphyromonas gingivalis can exhibit resistance to Taxol and have higher metastatic potential. Thus, in some embodiments, treating and eliminating P.g. with the APs improves multiple primary, secondary and adjuvant related cancer treatments. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some embodiments, the condition, disorder or disease to be treated by the present methods is a lung disease, such as non-smokers lung cancer and aspiration pneumonia. In some situations, targeting inflammation with anti-inflammatory therapy can lead to a significantly lower rate of recurrent cardiovascular events independent of lipid-level lowering. There can be a substantial lowering of non-smokers lung cancer with anti-inflammatory therapy targeting the interleukin-lb innate immunity pathway leading to significantly lower cancer mortality consistent with experimental data relating to interleukin-1b. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some situations, Porphyromonas gingivalis is the primary microbial pathogen as single source driver of inflammation and it's multiple NLRP3/IL-1β pathway mediated diseases including Atherosclerosis and Cardiovascular disease. In some situations, Infection with P. gingivalis can trigger the activation of NLRP3 and AIM2 inflammasomes via TLR2 and TLR4 signaling, leading to IL-1β secretion and pyroptic cell death. In addition, P. gingivalis-induced NLRP3 inflammasome activation can be dependent on ATP release, K+ efflux, and cathepsin B.

Without being bound by theory, the periodontopathogen Porphyromonas gingivalis has been shown to have several mechanisms of modulating innate immunity by limiting the activation of the NLRP3 inflammasome. The innate immune system can be the first line of defense against microbial pathogens. P. gingivalis can modify innate immunity by affecting inflammasome activity.

Wild type challenge of apolipoprotein E-deficient, spontaneously hyperlipidemic (ApoE) mice with P. gingivalis can increase IL-1β, IL-18, and TNF-a production in peritoneal macrophages and gingival or aortic gene expression of the NOD-like receptor family, NLRP3, IL-1β, pro-IL-1β and pro-caspase-1.

In some situations, outer membrane vesicles derived from Porphyromonas gingivalis can induce cell death with disruption of tight junctions in human lung epithelial cells. P. gingivalis OMVs can cause cell damage with cell membrane destruction in Human lung epithelial cell. P. gingivalis OMVs suppressed cell viability of Human lung epithelial cell by causing apoptosis. P. gingivalis OMVs translocated through oral cavity may be a trigger for inflammation of airway diseases. Thus, APs to this target can be used to address this in some embodiments. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some situations, P. gingivalis OMVs can induce cell death by destroying the barrier system in lung epithelial cells. P. gingivalis OMVs may be a factor in the engagement of periodontitis with respiratory system diseases. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some situations, Porphyromonas gingivalis is an aggravating factor for chronic obstructive pulmonary disease patients with periodontitis. The microbial analysis of sputum from COPD patients with CP to detect periodontal pathogen Porphyromonas gingivalis (P. gingivalis) both before and after nonsurgical periodontal therapy. A decrease in the count of P. gingivalis and decreased periodontal indices values can be observed in COPD patients with periodontitis after nonsurgical periodontal therapy. Lung function test (forced expiratory volume in the first/forced vital capacity) can be improved in COPD patients with periodontitis after nonsurgical periodontal therapy. In some embodiments, nonsurgical periodontal therapy can be a part of treatment protocol in COPD patients because it helps in reducing the P. gingivalis count and improves the lung function.

In some situations, gingipains are factors in the development of aspiration pneumonia caused by Porphyromonas gingivalis. Aspiration pneumonia can be a life-threatening infectious disease often caused by oral anaerobic and periodontal pathogens such as Porphyromonas gingivalis. This organism can produce proteolytic enzymes, known as gingipains, which can manipulate innate immune responses and promote chronic inflammation. P. gingivalis W83 gingipains can have a role in bronchopneumonia, lung abscess formation, and inflammatory responses. Gingipains can be important for clinical symptoms and infection-related mortality. Pathologies caused by wild-type (WT) P. gingivalis W83, including hemorrhage, necrosis, and neutrophil infiltration, can be absent from lungs infected with gingipain-null isogenic strains or WT bacteria preincubated with gingipain-specific inhibitors. Damage to lung tissue can be correlated with systemic inflammatory responses, as manifested by elevated levels of TNF, IL-6, IL-17, and C-reactive protein. These effects can be dependent on gingipain activity. Gingipain activity can also be implicated in the observed increase in IL-17 in lung tissues. Furthermore, gingipains can increase platelet counts in the blood and activated platelets in the lungs. Arginine-specific gingipains can make a greater contribution to P. gingivalis-related morbidity and mortality than lysine-specific gingipains. Thus, inhibition of gingipain may be a useful adjunct treatment for P. gingivalis-mediated aspiration pneumonia.

One of the pathogenic outcomes of P. gingivalis-triggered aspiration pneumonia can be thrombocytosis. Thrombocytosis can be associated with inflammatory disease, and the platelet count can be an acute-phase response to inflammation induced by P. gingivalis.

Animals challenged with WT P. gingivalis can show a sharp increase in TNF-α, IL-6, and MCP1 levels. The lungs from infected animals can show clear increases in MPO levels, which are indicative of neutrophil infiltration. The highest MPO concentrations can be detected in lung homogenates from animals infected with WT P. gingivalis, whereas those from mice infected with the ΔKgp and ΔRgp strains can show significantly lower MPO activity.

Intratracheal inoculation with either WT P. gingivalis or ΔKgp can lead to a significant increase in IL-17 expression in lung tissue and peripheral blood. Proteolytically active gingipains can modulate the course of P. gingivalis-associated aspiration pneumonia and aggravate the host immune response. P. gingivalis-derived enzymes can play an important role not only during chronic disease (e.g. periodontitis) but also during acute, life-threatening pneumonia. In some situations, TLR2 is implicated in Early Innate Immune Response to Acute Pulmonary Infection with Porphyromonas gingivalis in Mice. The periodontal pathogen Porphyromonas gingivalis is implicated in certain systemic diseases including atherosclerosis and aspiration pneumonia. This organism can induce innate responses predominantly through TLR2, which also mediates its ability to induce experimental periodontitis and accelerate atherosclerosis. TLR2-deficient mice can elicit reduced proinflammatory or antimicrobial responses (KC, MIP-1, TNF-, IL-6, IL-12p70, and NO) in the lung and exhibited impaired clearance of P. gingivalis compared with normal controls. However, the influx of polymorphonuclear leukocytes into the lung and the numbers of resident alveolar macrophages (AM) can be comparable between the two groups. TLR2 signaling can be important for in vitro killing of P. gingivalis by polymorphonuclear leukocytes or AM and, moreover, the AM bactericidal activity can require NO production. Strikingly, AM can be more potent than peritoneal or splenic macrophages in P. gingivalis killing, attributed to diminished AM expression of complement receptor-3 (CR3), which is exploited by P. gingivalis to promote its survival. Without being bound by theory, the selective expression of CR3 by tissue macrophages and the requirement of TLR2 inside-out signaling for CR3 exploitation by P. gingivalis indicates that the role of TLR2 in host protection may be contextual. In some embodiments, TLR2 may mediate destructive effects, as seen in models of experimental periodontitis and atherosclerosis, and the same receptor can confer protection against P. gingivalis in acute lung infection.

P. gingivalis can be a common isolate from aspiration pneumonia, which is usually seen in the elderly or the immunocompromised host and is epidemiologically associated with periodontal disease.

In some situations, periodontopathic anaerobes are involved in aspiration pneumonia. Porphyromonas gingivalis and Treponema denticola can coexist in chronic periodontitis lesions. In some situations, a mixed culture of P. gingivalis and T. denticola can be inoculated into the mouse trachea; and cause an infection inducing inflammatory cytokine production and pneumonia. In another series of investigations, professional oral health care (POHC), mainly cleansing administered by dental hygienists once a week for 24 months to elderly persons requiring daily care, can result in the reduction of the number of total anaerobes, Candida albicans, and Staphylococcus species and in the number of cases of fatal aspiration pneumonia. The POHC treatment of elderly persons for 6 months in the winter season can reduce the salivary levels of protease, trypsin-like activity, and neuraminidase and also can decrease the frequency of influenza cases.

In some embodiments, Porphyromonas gingivalis can induce inflammatory responses and promote apoptosis in lung epithelial cells infected with H1N1 via the Bcl-2/Bax/Caspase-3 signaling pathway. P. gingivalis may induce the production of a large number of inflammatory cytokines in lung epithelial cells. Lung epithelial cells infected with H1N1 and P. gingivalis can lead to the promoted production of inflammatory cytokines and the expression of iNOS, which may have also increased the accumulation of NO, resulting in an increased proportion of lung epithelial cells undergoing apoptosis via the Bcl-2/Bax/caspase-3 signaling pathway. Following BEAS-2B cell infection with P. gingivalis and H1N1, the concentrations of TNF-α, IL-1β and IL-6 in the supernatant can be significantly increased at each time point, compared with the H1N1 and P. gingivalis alone groups. These results demonstrated that lung epithelial cells infected with H1N1 and P. gingivalis can promote the production of inflammatory cytokines.

In some situations, Porphyromonas gingivalis modulates Pseudomonas aeruginosa-induced apoptosis of respiratory epithelial cells through the STAT3 signaling pathway. P. gingivalis invasion can transiently inhibit P. aeruginosa-induced apoptosis in respiratory epithelial cells via the signal transducer and activator of transcription 3 (STAT3) signaling pathway. The activated STAT3 can up-regulate the downstream anti-apoptotic moleculars survivin and B-cell leukemia-2 (bcl-2). This process can be accompanied by down-regulation of pro-apoptosis molecular Bcl-2-associated death promoter (bad) and caspase-3 activity inhibition. In addition, the activation of the STAT3 pathway can be affected by P. gingivalis in a dose-dependent manner. Finally, co-invasion of P. aeruginosa and P. gingivalis can lead to greater cell death compared with P. aeruginosa challenge alone. These results indicate that regulation of P. aeruginosa-induced apoptosis by P. gingivalis can contribute to the pathogenesis of respiratory disease. Thus, APs of the present disclosure targeting P. gingivalis can be used to address these disorders, conditions or diseases in some embodiments.

In some embodiments, oral cancer cells sustainedly infected with Porphyromonas gingivalis can exhibit resistance to Taxol and can have higher metastatic potential. Sustained infection with P. gingivalis, a major pathogen responsible for chronic periodontitis, can promote distant metastasis of oral cancer, as well as its resistance to anti-cancer agents. Thus, APs of the present disclosure targeting P. gingivalis can be used to address these disorders, conditions or diseases in some embodiments.

In some embodiments, the condition, disorder or disease treated by the present methods is Glioma. Without being bound by theory, Cathepsin B plays a critical role in inducing Alzheimer's Disease-like phenotypes following chronic systemic exposure to lipopolysaccharide from Porphyromonas gingivalis in mice. In some cases, systemic exposure to LPS from Porphyromonas gingivalis can induce AD-like phenotypes; Cathepsin B is implicated in inducing microglia-mediated neuroinflammation; Cathepsin B is implicated in inducing microglia-dependent AP accumulation in neurons. In some situations, a strong association can exist between periodontitis and accelerated cognitive decline in Alzheimer's disease (AD). Cathepsin (Cat) B can play a critical role in the initiation of neuroinflammation and neural dysfunction following chronic systemic exposure to lipopolysaccharide from Porphyromonas gingivalis (PgLPS). Thus, APs of the present disclosure targeting P. gingivalis can be used to address these disorders, conditions or diseases in some embodiments.

In some embodiments, the condition, disorder or disease is a gut microbiome-related disorder. A variety of gut microbiome-related disorder can be treated by the APs of the present disclosure. In some embodiments, the gut microbiome-related disorder is an intestinal disorder such as, without limitation, inflammatory bowel disease, irritable bowel syndrome (IBS), coeliac disease. In some embodiments, the gut microbiome-related disorder is an extra-intestinal disorder such as, without limitation, allergy, asthma, metabolic syndrome, cardiovascular disease, and obesity. Without being limited by theory, endotoxemia that may cause metabolic disorders can be related to changes in the gut microbiota caused by oral bacteria, e.g., P. gingivalis. In some cases, periodontal inflammation can affect the mechanical and immune barrier functions of the gut. Orally administered P. gingivalis can cause composition shifts in the gut microbiota and increase serum endotoxin and inflammatory markers, and affect the gut immune system. In addition, P. gingivalis has been associated with NAFLD and non-alcoholic steatohepatitis (NASH). P. gingivalis can be detected in the gut of the NAFLD and NASH patients. Thus, APs of the present disclosure targeting P. gingivalis can be used to address these disorders, conditions or diseases in some embodiments.

In some embodiments, the condition, disorder or disease is a cognitive disorder. In some embodiments, the condition, disorder or disease is dementia associated with microvasculature defects. In some embodiments, the condition, disorder or disease is microvascular defects Parkinson's. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some situations, cerebral oxidative stress and microvasculature defects are implicated in TNF-α Expressing Transgenic and Porphyromonas gingivalis-Infected ApoE−/− Mice. There can be a major difference in the hippocampi of P. gingivalis-infected and sham-infected ApoE−/− mice, in terms of increased protein carbonyl/oxidized protein content in the hippocampal micro-vasculature. Hippocampal microvascular structures and the homeostasis of the brain can be at risk from elevated oxidative stress and oxidative protein damage, following P. gingivalis infection. Without being bound by theory, following recurrent episodes of active periodontal disease, there exists a possibility for the development of a defective BBB, post neuroinflammation-mediated cerebral parenchymal tissue injury. The rising levels of intrinsic and extrinsic sources of cytokines, oxidative stress, and developing BBB defects may be implicated as early modifiers of neurodegenerative and disease severity leading to deteriorating memory. Infection with P. gingivalis can be interpreted as one of the plausible mechanisms by which a susceptible host can develop dementia.

A variety of cognitive disorders can be treated by the APs of the present disclosure. In some embodiments, the cognitive disorder is Alzheimer's disease (AD). Without being bound by theory, periodontitis has been shown to be a risk factor for AD and a more rapid cognitive decline. In some cases, genetic predisposition, P. gingivalis infection and microglia could promote neurodegeneration typical of that reported for AD. P. gingivalis specific cell free DNA can be detected in the cerebrospinal fluid of AD patients and the pathogen's protease virulence factors, arginine-gingipain (Rgp) and lysine-gingipain (Kgp), can be found in the brains of over 90% of AD patients and can correlate with tau and ubiquitin pathology. P. gingivalis can invade and persist in mature neurons, which, once infected, can display signs of AD-like neuropathology, including the accumulation of autophagic vacuoles and multivesicular bodies, cytoskeleton disruption, an increase in phosphotau/tau ratio, and synapse loss. Gingipains of P. gingivalis can digest tau protein into peptide fragments, some of which include tau residues prone to phosphorylation and some of which include two of the four microtubule binding domains that form paired/straight helical filaments constituting neurofibrillary tangles (NFTs). In some cases, Gingipains have been found to be neurotoxic in vivo and in vitro, having detrimental effects on tau. P. gingivalis lipopolysaccharide (LPS) can activate the phosphoinositide 3-k inase/Akt (PI3K/AKT) pathway and increase expression of glycogen synthase kinases-3 beta (GSK-3β), which can phosphorylate tau. P. gingivalis can invade and survive in neurons and generate intra-neuronal gingipains that are proteolytically active, leading to neurodegeneration associated with AD. In some embodiments, a subject with Down's syndrome is at increased risk of developing AD. In some embodiments, the AP comprises, consists, or consists essentially of a sequence having at least about 75%, at least about 80%, at least about 85%, at least about 90%, at least about 95%, at least about 99%, and/or at about 100% identity to the sequence YTYTVYRDGTKIK (SEQ ID NO: 6).

In some cases, P. gingivalis can induce migration of microglial cells to sites of infection in the brain, through activation of mitogen-activated protein kinase/extracellular signal-regulated kinase (ERK) kinase/ERK pathway. P. gingivalis can induce synthesis of matrix metalloproteinases (MMPs), which can have an important role in neuroinflammatory disorders including AD. Oral infection with P. gingivalis can result in the pathogen spreading to the brain and activating microglia. P. gingivalis can down-regulate TREM-2 expression in microglia. Lack of TREM-2 protein may accelerate aging processes, neuronal cell loss and reduce microglial activity leading to neuroinflammation. P. gingivalis can contribute to development of AD inflammatory pathology through mechanisms involving acute phase proteins, cytokines and the complement cascade where neurons would be attacked. Inappropriate complement activity can play a significant role in AD pathophysiology.

LPS, a virulence factor of P. gingivalis, in the brain can initiate neuroinflammation in the form of microglial cell activation, and the neuroinflammatory response can be stronger with age. Age-associated priming of microglia may have a role in exaggerated inflammation induced by activation of the peripheral immune system. In some cases, P. gingivalis can cause an imbalance in M1/M2 activation in macrophages, resulting in a hyperinflammatory environment that promotes the pathogenesis of periodontitis, and leptomeningeal cells can transduce inflammatory signals from peripheral macrophages to brain resident microglia exposed to P. gingivalis LPS. In microglia, P. gingivalis LPS can increase the production of cathepsin B and pro-forms of caspase-1 and IL-1β through activation of Toll-Like Receptor (TLR) 2/NF-kB signaling. Cathepsin B is implicated in in P. gingivalis LPS-induced AD-like pathology, and may be necessary for the induction of AD-like pathology following chronic systemic exposure to P. gingivalis LPS. In some cases, treating periodontitis can lead to improvements in cognition. A chronic infection of the brain with P. gingivalis can cause serious consequences for the BBB and subsequent mental health. Thus, APs of the present disclosure targeting P. gingivalis can be used to address these disorders, conditions or diseases in some embodiments.

In some embodiments, the condition, disorder or disease is an age-related disorder. Without being bound by theory, P. gingivalis can impact cellular biochemical pathways that are associated with improved longevity or shortened life spans, e.g., by regulating autophagy and apoptosis, modulating the mTORC1 pathway, or targeting cellular senescence by selectively eliminating senescent cells. Disrupted autophagy has been linked to numerous diseases including Parkinson's disease, and type 2 diabetes. In some cases, P. gingivalis minor (Mfa1) fimbriae can manipulate dendritic cell (DC) signaling to perturb both autophagy and apoptosis. Mfa1 can induce Akt nuclear localization and activation, and ultimately can induce mTOR in DCs. P. gingivalis can promote DC survival by increasing anti-apoptotic Bc12 protein expression and decreasing pro-apoptotic proteins Bim, Bax and cleaved caspase-3. In some cases, lipophilic outer membrane vesicles (OMV) shed from P. gingivalis can promote monocyte unresponsiveness to live P. gingivalis. Full reactivity to P. gingivalis can be restored by inhibition of mTOR signaling, which can promote Toll-like receptor 2 and Toll-like receptor 4 (TLR2/4)-mediated tolerance in monocytes. Without being bound by theory, it is thought that P. gingivalis, a facultative intracellular microbe, may damage not only cell membranes but also the mitochondrion, triggering a bioenergetic crisis and NLRP3-induced cellular senescence. Moreover, age-related brain LPS elevation may trigger intracellular iron migration, an innate immune response to withhold iron from pathogens.

Without being bound by theory, the major surface glycoproteins of P. gingivalis—Pgm6 and Pgm7, also called outer membrane protein A-like proteins (OmpALPs)—mediate resistance to the bactericidal activity of human serum, and specifically protect P. gingivalis from the bactericidal activity of LL-37 and from innate immune recognition by TLR4. LL-37 proteolysis by P. gingivalis may provide neighboring dental plaque species with resistance to LL-37, which in turn can benefit P. gingivalis. Thus, APs of the present disclosure targeting P. gingivalis can be used to address these disorders, conditions or diseases in some embodiments.

In some embodiments, the condition, disorder or disease is an aneurysm, e.g., cerebral or abdominal aneurysm. In some cases, pro-inflammatory response elicited by Porphyromonas Gingivalis lipopolysaccharide exacerbates the rupture of experimental cerebral aneurysms. Porphyromonas gingivalis LPS can exacerbate vascular inflammation and can enhance the rupture of intracranial aneurysms.

In some situations, CPI can be significantly higher in patients with IAs than the controls (2.7 vs 1.9, p<0.05) and their DNA level of subgingival plaques and their plasma IgG titers of Pg can also be higher. Periodontal disease can be more severe and the plasma IgG titers of Pg can be higher in patients with ruptured- than unruptured IAs, suggesting that Pg is associated not only with the formation but also the rupture of IAs. Severe periodontal disease and Pg infection may be involved in the pathophysiology of IAs.

In some situations, the condition, disorder or disease is depression. Without being bound by theory, it is thought Porphyromonas gingivalis can induce depression via downregulating p75NTR-mediated BDNF maturation in astrocytes. In some embodiments, Pg-LPS decreases the level of astrocytic p75NTR and then downregulates BDNF maturation, leading to depression-like behavior in mice. Pg can be a modifiable risk factor for depression. In some embodiments, Porphyromonas gingivalis (Pg) can induce depression-like behaviors; Astrocytic p75NTR can be decreased in Pg-colonized mice; Overexpression of p75NTR in astrocytes can rescue depressive behaviors; Antibiotic therapy can ameliorate Pg-induced depressive behavior in mice. Thus, APs of the present disclosure targeting P. gingivalis can be used to address these disorders, conditions or diseases in some embodiments.

In some embodiments, the condition, disorder or disease is peri-implantitis. In some situations, oral infection with Porphyromonas gingivalis can induce peri-implantitis, and can be implicated in bone loss and the local inflammatory response. Porphyromonas gingivalis infection can induce greater bone loss around implants than around teeth. In non-infected animals, the presence of the implant can correlate with elevated expression of Il-10, Foxp3 and Rank1/Opg ratio, while Tnf-α levels can be decreased relative to tissue around teeth. Six weeks following infection, Tnf-α can be increased significantly while the expression of Foxp3 can be decreased in the tissue around the implants. Oral infection with P. gingivalis of mice with implants can induce bone loss and a shift in gingival cytokine expression. In some situations, the fimA type Ib genotype of P. gingivalis can play a role in the destruction of peri-implant tissue, indicating that it may be a distinct risk factor for peri-implantitis.

In some situations, biocorrosion of pure and SLA titanium surfaces is observed in the presence of Porphyromonas gingivalis and can have effects on osteoblast behavior. P. gingivalis can colonize on the pure and SLA titanium surfaces and weaken their surface properties, especially a decrease in the protective TiO2 film, which can induce the biocorrosion and further negatively affected the osteoblast behavior.

In some situations, titanium can have an influence on in vitro fibroblast-Porphyromonas gingivalis interaction in peri-implantitis. Higher doses of TiO₂ can be toxic to PIGFs and in sub-toxic doses, TiO2 can cause an increase in gene expression of tumour necrosis factor (TNF)-A and increase protein production of TNF-α, interleukin (IL)-6 and IL-8. A challenge with P. gingivalis alone can induce gene expression of TNF-A, IL-1β, IL-6 and IL-8. A combined challenge with TiO₂ and P. gingivalis can cause a stronger increase in gene expression of TNF-A and protein production of TNF-α and MCP-1 than P. gingivalis alone. TiO₂ particles and P. gingivalis, individually, can induce pro-inflammatory responses in PIGFs. Furthermore, TiO₂ particles and viable P. gingivalis can further enhance gene expression and production of TNF-α by PIGFs. Without being bound by theory, Ti wear particles in the peri-implant tissues in combination with P. gingivalis infection may contribute to the pathogenesis of peri-implantitis by enhancing the inflammation in peri-implant tissues.

In some situations, cytokine and matrix metalloproteinase expression in fibroblasts from peri-implantitis lesions can be observed response to viable Porphyromonas gingivalis. Fibroblasts from peri-implantitis and periodontitis lesions can exhibit a more pronounced inflammatory response to the P. gingivalis challenge than fibroblasts from healthy donors. Without being bound by theory, they may therefore be involved in the development of inflammation in peri-implantitis and periodontitis. Moreover, the sustained upregulation of inflammatory mediators and MMP-1 in peri-implantitis fibroblasts may play a role in the pathogenesis of peri-implantitis.

In some embodiments, the condition, disorder or disease is bone loss or osteoporosis. In some cases periodontal disease and associated bone loss by Porphyromonas gingivalis Stimulates bone resorption by enhancing RANKL (Receptor Activator of NF-κB Ligand) through Activation of Toll-like Receptor 2 in Osteoblasts. LPS P. gingivalis and Pam2 can enhance osteoclast formation in periosteal/endosteal cell cultures by increasing RANKL. LPS P. gingivalis and Pam2 can also up-regulate RANKL and osteoclastic genes in vivo, resulting in an increased number of periosteal osteoclasts and immense bone loss in wild type mice but not in Tlr2-deficient mice. In some cases, LPS P. gingivalis can stimulate periosteal osteoclast formation and bone resorption by stimulating RANKL in osteoblasts via TLR2. Without being bound by theory, this effect may be important for periodontal bone loss and for the enhanced bone loss seen in rheumatoid arthritis patients with concomitant periodontal disease. In some situations, activation of TLR2 in osteoblasts by P. gingivalis increases RANKL production, osteoclast formation, and bone loss both ex vivo and in vivo. P. gingivalis can stimulate alveolar bone loss can cause a more severe loss of juxta-articular bone in RA. In some situations, TLR2, which is highly expressed in RA synovium, is not only activated by pathogen-associated molecular patterns such as P. gingivalis but also by endogenous ligands present in RA synovium such as gp96 and Snapin. There may be a role of endogenous ligands in the pathogenesis of RA bone erosions. Moreover, genetic or antibody-mediated inactivation of TLR2 can reduce cytokine production in P. gingivalis-stimulated neutrophils or macrophages, suggesting that TLR2 plays a non-redundant role in the host response to P. gingivalis. In the absence of MyD88, inflammatory TLR2 signaling in P. gingivalis-stimulated neutrophils or macrophages can depend upon PI3K. TLR2-PI3K signaling may be implicated in P. gingivalis evasion of killing by macrophages, since their ability to phagocytose this pathogen can be reduced in a TLR2 and PI3K-dependent manner. Moreover, within those cells that did phagocytose bacteria, TLR2-PI3K signaling can block phago-lysosomal maturation, thereby revealing a novel mechanism whereby P. gingivalis can enhance its intracellular survival. In some cases, P. gingivalis can uncouple inflammation from bactericidal activity by substituting TLR2-PI3K in place of TLR2-MyD88 signaling. P. gingivalis can be a keystone pathogen, which can manipulate the host inflammatory response in a way that promotes bone loss but not bacterial clearance. Without being bound by theory, modulation of these host response factors may be a therapeutic approach to improve outcomes in disease conditions associated with P. gingivalis.

In some cases, periodontal pathogenic bacteria as well as intestinal dysbiosis are involved in the determinism of bone mineral density BMD loss, and contribute to the onset and worsening of osteoporosis OP. Thus, APs of the present disclosure targeting P. gingivalis can be used to address these disorders, conditions or diseases in some embodiments.

In some situations, early host-microbe interaction is implicated in a peri-implant oral mucosa-biofilm model. In some situations, various factors (V. dispar, P. gingivalis, immune cells) could be involved in the disruption or maintenance of homeostasis. Thus, APs of the present disclosure targeting P. gingivalis can be used to address these disorders, conditions or diseases in some embodiments.

In some embodiments, a subject has been found to have detectable levels of gingipains associated with P. gingivalis such as Rgp and Kgp in the blood that may be eliminated with a method of the present disclosure in order to maintain wellness. In some embodiments, the wellness can be maintained through the optimization of the gut biome, prevention, initiation or progression of conditions such as vascular inflammation or other disease states to the point of clinical symptoms. In some embodiments, the method includes retreatment of the subject with the APs. In some embodiments, the method includes obtaining one or more measures of blood borne gingipains associated with P. gingivalis to determine whether the subject requires retreatment with the AP. Thus, APs of the present disclosure targeting P. gingivalis can be used to address these disorders, conditions or diseases in some embodiments.

In some embodiments methods of the recent disclosure include administering to the subject an AP of the present disclosure in conjunction with one or more treatments of telomer length and/or prevention with various drugs and or natural supplements. Without being bound by theory, it has been shown that shorter telomere lengths are associated with a diagnosis of periodontitis and their measures correlate with the oxidative stress and severity of disease. Thus, APs of the present disclosure targeting P. gingivalis can be used to address these disorders, conditions or diseases in some embodiments.

Within the above “Various Applications” section, there is an extensive discussion of various scientific aspects regarding various indications. As a point of clarity, unless denoted otherwise in some manner (such as their presence elsewhere, in the Examples, or in the figures or other detailed account), this summary of results is provided for understanding of the present disclosure and is not meant to denote an experiment reduced to practice by the present inventors.

Also provided herein are methods of preventing one or more conditions, disorders, or diseases, as disclosed herein, by administering to a subject, e.g., a subject at risk of developing the condition, disorder, or disease, an effective amount of an AP of the present disclosure, to thereby prevent the condition, disorder, or disease or developing. In some embodiments, the subject is predisposed to developing the condition, disorder, or disease. In some embodiments, the subject has a past history of an P. gingivalis infection and/or condition or disease associated with a P. gingivalis infection, as disclosed herein. In some embodiments, the subject is genetically predisposed to develop the condition, disorder, or disease. In some embodiments, the method includes identifying a subject predisposed to developing any one or more of the conditions, disorders, or diseases, as disclosed herein, and administering to the subject an effective amount of an AP of the present disclosure to thereby prevent, reduce the likelihood and/or delay the onset of the conditions, disorders, or diseases.

In any of the above methods, the AP can be administered in conjunction with one or more additional therapeutic agents for treating or preventing the condition, disease or disorder. In some embodiments, a therapeutic agent for treating or preventing the condition, disease or disorder, as disclosed herein, can be administered to a subject in need thereof at a therapeutically effective amount, and an effective amount of the AP of the present disclosure can be administered to the subject. Administration of the AP can in some embodiments improve or enhance the therapeutic effect of the other therapeutic agent. As used herein, a first agent administered in conjunction with administering a second agent can include administering the first agent before, after, or simultaneously as the second agent. In some embodiments, the first agent and second agent are administered within an interval such that the therapeutic effect of the first agent is present in the subject when the second agent is administered to the subject.

By way of non-limiting examples, the AP can in some embodiments be administered in conjunction with one or more additional therapeutic agents for treating or preventing a vascular disease, as disclosed herein. In some embodiments, the other therapeutic agent includes a serum lipid lowering agent. Any suitable serum lipid lowering agent can be used. In some embodiments, the serum lipid lowering agent includes, without limitation, statins (e.g., atorvastatin, cerivastatin, fluvastatin, lovastatin, mevastatin, pitavastatin, pravastatin, rosuvastatin, simvastatin), Nicotinic acid (Niacin) (e.g., NIACOR, NIASPAN (slow release niacin), SLO-NIACIN (slow release niacin), CORDAPTIVE (laropiprant)), Fibric acid (e.g., LOPID (Gemfibrozil), TRICOR (fenofibrate), Bile acid sequestrants (e.g., QUESTRAN (cholestyramine), colesevelam (WELCHOL), colestipol (COLESTID)), Cholesterol absorption inhibitors (e.g., ZETIA (ezetimibe)), PPAR gamma agonsits, PPAR alpha/gamma agonists, squalene synthase inhibitors, CETP inhibitors, anti-hypertensives, anti-diabetic agents (such as sulphonyl ureas, insulin, GLP-1 analogs, DDPIV inhibitors, e.g., metaformin), ApoB modulators, such as mipomersan, MTP inhibitoris and/or arteriosclerosis obliterans treatments.

The AP can in some embodiments be administered in conjunction with one or more additional therapeutic agents for treating or preventing cancer, as disclosed herein. In some embodiments, the other therapeutic agent includes an anti-cancer therapeutic that is a small molecule drug or immunotherapeutic agent. Any suitable small molecule drug or immunotherapeutic agent can be used.

In some embodiments, a dosing strategy for therapeutics can optimize the therapeutic outcome by minimizing adverse effects and maximizing efficacy across the target patient population. Multiple factors including pharmacokinetics, pharmacodynamics, exposure-response (efficacy/safety) relationships, disease burden, patient characteristics, compliance and pharmaco-economics can affect the decision on the clinical dose and dose regimen. In some embodiments, a consideration here is whether patients should be dosed based on body size, or whether body size-independent (fixed) dosing offers a viable alternative. The dosing strategy can vary. In some embodiments, body size based dosing (i.e. a dose proportional to the body size) can be used. In some embodiments, this dosing approach can reduce inter-subject variability in drug exposure, and controlling for this pharmacokinetic variability in turn can significantly reduce variability in the response to drug treatment across the population. In some embodiments, doses are based on body size. In some embodiments, body size-based dosing is used when there is a statistically significant body size effect on pharmacokinetic parameter(s) in the population pharmacokinetic analysis.

For systemic administration, subjects can be administered a therapeutic amount of the AP in the microgram to milligram range or more, or an amount in a range defined by any two of the preceding values.

In some embodiments, the AP can be a recombinant protein. A recombinant protein (rGP-1) (FIG. 4) was engineered to contain a fragment of the HagA gingipain containing a single copy of the epitope recognized by KB001. This rGP-1 is expressed as a fusion protein with glutathione S transferase (GST) to increase solubility when expressed in E. coli bacterial cells. rGP-1 bears a C-terminal hexahistidine tag for purification and a proteolytic site recognized by the tobacco etch virus (TEV) upstream of the gingipain fragment to permit removal of the GST fusion partner after purification.

In some embodiments, the vaccine sequence can include some or all of the sequence of the gingipain/vft fragment shown below:

(SEQ ID NO: 24)
DPSCSPTNMIMDGTASVNIPAGTYDFAIAAPQANAKIWIAGQGPTKEDDY
VFEAGKKYHFLMKKMGSGDGTELTISEGGGSDYTYTVYRDGTKIKEGLTA
TTFEEDGVAAGNHEYCVEVKYTAGVSPKVCKDVTVEGSNEFAPVQNLTGS
AVGQKVTLKWDAPNGHHHHHH-.

In some embodiments, the peptide need not include the histidine tag or all of it. In some embodiments, the peptide includes the sequence above or that in FIG. 4 or 5A and/or 5B and/or 5C. In some embodiments, the peptide is at least 80, 85, 90, 95, 96, 97, 98, 99% identical or similar to the sequence above or in FIG. 4 or 5A and/or 5B and/or 5C. In some embodiments, the variants provided herein (e.g., of the above sequences or SEQ ID NO: 6, etc.) can be at least 80, 85, 90, 95, 96, 97, 98, or 99% identical, with any variations being conservative substitutions.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### Arrangements

The following arrangements (first and second arrangements) are further contemplated as various embodiments:

### First Arrangements

1. An antigenic composition comprising at least one isolated and purified peptide and/or protein, wherein the isolated and purified protein consists of

(SEQ ID NO: 1)
GVSPKVCKDVTVEGSNEFAPVQNLT
and/or
(SEQ ID NO: 2)
YCVEVKYTAGVSPK,
(SEQ ID NO: 3)
AGTYDFAIAAPQANAKIWIAGQGPTKEDDYVFEAGKKYHFLMKKMGSGDG
TELTISEGGGSDYTYTVYRDGTKIKEGLTATTFEEDGVAAGNHEYCVEVK
YTAGVSPKVCKDVTVEGSNEFAPVQNLT,
and/or
(SEQ ID NO: 4)
GVSPK

or an immunogenic fragment or variant thereof.

2. An antigenic composition comprising a conformational epitope defined by the sequence YCVEVKYTAGVSPK (SEQ ID NO: 2), wherein a structure of the confirmational epitope is effectively the same as a structure of YCVEVKYTAGVSPK (SEQ ID NO: 2) within a sequence of Kgp.

3. An antigenic composition comprising a conformational epitope defined by the sequence GVSPKVCKDVTVEGSNEFAPVQNLT (SEQ ID NO: 1), wherein a structure of the confirmational epitope is effectively the same as a structure of GVSPKVCKDVTVEGSNEFAPVQNLT (SEQ ID NO: 1) within a sequence of Kgp.

4. An antigenic composition comprising a conformational epitope comprising one or more amino acid within GVSPK (SEQ ID NO: 4), wherein a structure of the confirmational epitope is effectively the same as a structure of a GVSPK fragment when located within YCVEVKYTAGVSPK (SEQ ID NO: 2).

5. An antigenic composition comprising a conformational epitope comprising one or more amino acids within GVSPK (SEQ ID NO: 4), wherein a structure of the confirmational epitope is effectively the same as a structure of a GVSPK fragment when located within a full length sequence of Kgp.

6. An antigenic composition comprising a conformational epitope defined by the sequence AGTYDFAIAAPQANAKIWIAGQGPTKEDDYVFEAGKKYHFLMKKMGS GDGTELTIS EGGGSDYTYTVYRDGTKIKEGLTATTFEEDGVAAGNHEYCVEVKYTAGVSPKVCK DVTVEGSNEFAPVQNLT (SEQ ID NO: 3), wherein a structure of the confirmational epitope is effectively the same as a structure of AGTYDFAIAAPQANAKIWIAGQGPTKEDDYVFEAGKKYHFLMKKMGS GDGTELTIS EGGGSDYTYTVYRDGTKIKEGLTATTFEEDGVAAGNHEYCVEVKYTAGVSPKVCK DVTVEGSNEFAPVQNLT (SEQ ID NO: 3), within a sequence of Kgp.

7. An antigenic composition comprising or consisting of a polypeptide of at least one of:
a sequence of GVSPK (SEQ ID NO: 4),
a sequence of GVSPK (SEQ ID NO: 4) having 1, 2, or 3 conservative substitutions,
a sequence of GVSPK (SEQ ID NO: 4) having 1, or 2 substitutions, but having a similar structure to that of GVSPK when contained within a full length sequence of Kgp,
a sequence YCVEVKYTAGVSPK (SEQ ID NO: 2),
having at least 80% identity to SEQ ID NO: 2,
an immunogenic fragment of 8, 9, 10, 12, 13, or more consecutive amino acids of SEQ ID NO: 2, and/or
a variant comprising a conservative substitution of at least one amino acid of SEQ ID NO: 2,
or a fragment thereof capable of raising an immune response against P. gingivalis, and wherein said polypeptide is not complexed with other Arg-specific and Lys-specific P. gingivalis proteinase and adhesin proteins.

8. An isolated and purified polypeptide essentially consisting of a sequence YCVEVKYTAGVSPK (SEQ ID NO: 1).

9. A composition for use in raising an immune response directed against P. gingivalis in a subject, the composition comprising an effective amount of at least one amino acid sequence comprising at least 10 amino acids identical to a contiguous amino acid sequence of:

(SEQ ID NO: 2)
YCVEVKYTAGVSPK,

having at least 80% identity to SEQ ID NO: 2,

an immunogenic fragment of 8, 9, 10, 12, 13, or more consecutive amino acids of SEQ ID NO: 2, and/or

a variant comprising a conservative substitution of at least one amino acid of SEQ ID NO: 2, and a pharmaceutically acceptable carrier.

10. An immunogenic composition comprising:
at least one isolated polypeptide of 49 or fewer amino acids, said polypeptide comprising an amino acid sequence selected from the group consisting of:
a polypeptide of YCVEVKYTAGVSPK (SEQ ID NO: 2),
a polypeptide having at least 80% identity to SEQ ID NO: 2,
an immunogenic fragment of 8, 9, 10, 12, 13, or more consecutive amino acids of SEQ ID NO: 2, and
a variant comprising a conservative substitution of at least one amino acid of SEQ ID NO: 2, and/or
an immunological adjuvant.

11. An immunogenic composition comprising at least one isolated polypeptide, and a pharmaceutically acceptable carrier, wherein the at least one polypeptide is selected from the group consisting of Porphyromonas gingivalis protein YCVEVKYTAGVSPK (SEQ ID NO: 2), a polypeptide having at least 80% identity to SEQ ID NO: 2, an immunogenic fragment of 8, 9, 10, 12, 13, or more consecutive amino acids of SEQ ID NO: 2, and a variant comprising a conservative substitution of at least one amino acid of SEQ ID NO: 2.

12. The immunogenic composition of first arrangement 11, further comprising an adjuvant.

13. The immunogenic composition of first arrangement 12, wherein the adjuvant is an aluminum salt adjuvant.

14. The immunogenic composition of first arrangement 12, wherein the immunogenic composition is formulated for use orally.

15. The immunogenic composition of first arrangement 12, wherein the immunogenic composition is formulated for percutaneous administration.

16. An antigenic composition comprising at least one of:
a polypeptide consisting essentially of GVSPK (SEQ ID NO: 4)
a polypeptide of YCVEVKYTAGVSPK (SEQ ID NO: 2),
a polypeptide having at least 80% identity to SEQ ID NO: 2, and/or
an immunogenic fragment of 8, 9, 10, 12, 13, or more consecutive amino acids of SEQ ID NO: 2, and a variant comprising a conservative substitution of at least one amino acid of SEQ ID NO: 2.

17. The antigenic composition of first arrangement 16, wherein the peptide is no longer than 50 amino acids in length.

18. The antigenic composition of first arrangement 16, wherein the peptide is no longer than 20 amino acids in length.

19. The antigenic composition of first arrangement 16, wherein the peptide is no longer than 14 amino acids in length.

20. A method of treating a disease or condition associated with the presence of P. gingivalis in an oral tissue of a subject, comprising administering to the subject a composition of first arrangements 1-19 and administering to the subject a peptide, wherein the polypeptide comprises: YCVEVKYTAGVSPK, a polypeptide having at least 80% identity to this sequence, an immunogenic fragment of 8, 9, 10, 12, 13, or more consecutive amino acids of this sequence, and a variant comprising a conservative substitution of at least one amino acid of this sequence.

21. A method of administering the immunogenic composition of any one of first arrangements 1-19, the method comprising subgingivally administering the immunogenic composition to a subject.

22. The method of first arrangement 21, wherein the immunogenic composition is administered at least two times.

23. A method of treating or preventing a vascular disease or symptoms thereof, comprising:
identifying a subject in need of treating or preventing a vascular disease or symptoms thereof; and
administering to the subject a therapeutically effective amount of the immunogenic composition any one of first arrangements 1-19,
thereby treating or preventing the vascular disease or symptoms thereof.

24. The method of first arrangement 23, wherein the vascular disease comprises cardiovascular disease, atherosclerosis, coronary artery disease, myocardial infarction, stroke, and myocardial hypertrophy.

25. The method of first arrangement 23 or 24, further comprising administering to the subject at least one other therapeutic agent for treating or preventing the vascular disease, or symptoms thereof.

26. The method of first arrangement 25, wherein the other therapeutic agent comprises a serum lipid lowering agent.

27. The method of first arrangement 26, wherein the other therapeutic agent is a statin.

28. A method of treating or preventing a vascular disease or symptoms thereof, comprising:
administering to a subject in need of treating or preventing a vascular disease, or symptoms thereof, a therapeutically effective amount of at least one therapeutic agent for treating or preventing the vascular disease, or symptoms thereof; and
administering an effective amount of the immunogenic composition of any one of first arrangements 1-19, to thereby enhance the therapeutic effect of the at least one therapeutic agent.

29. The method of first arrangement 28, wherein the other therapeutic agent comprises a serum lipid lowering agent.

30. The method of first arrangement 29, wherein the other therapeutic agent is a statin.

31. A method of treating or preventing a systemic disease or symptoms thereof, comprising:
identifying a subject in need of treating or preventing a systemic disease or symptoms thereof, wherein the systemic disease is one or more of type II diabetes, insulin resistance and metabolic syndrome; and
administering to the subject a therapeutically effective amount of the immunogenic composition of any one of first arrangements 1-19,
thereby treating or preventing the systemic disease or symptoms thereof.

32. A method of treating or preventing rheumatoid arthritis or symptoms thereof, comprising:
identifying a subject in need of treating rheumatoid arthritis or symptoms thereof; and
administering to the subject a therapeutically effective amount of the immunogenic composition of any one of first arrangements 1-19,
thereby treating or preventing the rheumatoid arthritis or symptoms thereof.

33. A method of treating or preventing cancer or symptoms thereof, comprising:
identifying a subject in need of treating cancer or symptoms thereof; and
administering to the subject a therapeutically effective amount of the immunogenic composition of any one of first arrangements 1-19,
thereby treating or preventing the cancer or symptoms thereof.

34. The method of first arrangement 33, wherein the cancer is oral, gastrointestinal, lung or pancreatic cancer.

35. The method of first arrangement 33 or 34, further comprising administering to the subject at least one other therapeutic agent for treating or preventing the cancer, or symptoms thereof.

36. The method of first arrangement 35, wherein the other therapeutic agent comprises a small molecule drug or immunotherapeutic agent.

37. A method of treating or preventing cancer or symptoms thereof, comprising:
administering to a subject in need of treating or preventing cancer, or symptoms thereof, a therapeutically effective amount of at least one therapeutic agent for treating or preventing the cancer, or symptoms thereof; and
administering an effective amount of the immunogenic composition of any one of first arrangements 1-19, to thereby enhance the therapeutic effect of the at least one therapeutic agent.

38. The method of first arrangement 37, wherein the at least one therapeutic agent comprises a small molecule drug or immunotherapeutic agent.

39. The method of first arrangement 37 or 38, wherein the cancer is oral, gastrointestinal, lung or pancreatic cancer.

40. A method of treating or preventing a gut microbiome-related disorder or symptoms thereof, comprising:
identifying a subject in need of treating a gut microbiome-related disorder or symptoms thereof; and
administering to the subject a therapeutically effective amount of the immunogenic composition of any one of first arrangements 1-19,
thereby treating or preventing the gut microbiome-related disorder or symptoms thereof.

41. The method of first arrangement 40, wherein the gut microbiome-related disorder comprises inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity.

42. A method of treating or preventing a cognitive disorder or symptoms thereof, comprising:
identifying a subject in need of treating a cognitive disorder or symptoms thereof; and
administering to the subject a therapeutically effective amount of the immunogenic composition of any one of first arrangements 1-19,
thereby treating or preventing the cognitive disorder or symptoms thereof.

43. The method of first arrangement 42, wherein the cognitive disorder is Alzheimer's disease.

44. The method of first arrangement 42 or 43, wherein the cognitive disorder is early, middle or late dementia.

45. A method of treating or preventing an age-related or longevity-related disorder, or symptoms thereof, comprising:
identifying a subject in need of treating an age-related or longevity-related disorder; and
administering to the subject a therapeutically effective amount of the immunogenic composition of any one of first arrangements 1-19,
thereby treating or preventing the age-related or longevity-related disorder, or symptoms thereof.

46. A method of treating or preventing a post event myocardial hypertrophy or symptoms thereof, comprising:
identifying a subject in need of treating or preventing a post event myocardial hypertrophy or symptoms thereof; and
administering to the subject a therapeutically effective amount of the immunogenic composition of any one of first arrangements 1-19,
thereby treating or preventing the post event myocardial hypertrophy or symptoms thereof.

47. A method of treating a wound, comprising:
identifying a subject in need of treating a wound; and
administering to the subject a therapeutically effective amount of the immunogenic composition of any one of first arrangements 1-19,
whereby closure of the wound is enhanced, thereby treating the wound.

48. A method of treating or preventing an age-related macular degeneration (AMD) or symptoms thereof, comprising:
identifying a subject in need of treating or preventing AMD or symptoms thereof; and
administering to the subject a therapeutically effective amount of the immunogenic composition of any one of first arrangements 1-19,
thereby treating or preventing the AMD or symptoms thereof.

49. A method of treating or preventing an aneurysm or symptoms thereof, comprising:
identifying a subject in need of treating or preventing an aneurysm or symptoms thereof; and
administering to the subject a therapeutically effective amount of the immunogenic composition of any one of first arrangements 1-19,
thereby treating or preventing the aneurysm or symptoms thereof.

50. The method of first arrangement 49, wherein the aneurysm is a cerebral or abdominal aneurysm.

51. A method of treating or preventing a glioma or symptoms thereof, comprising:
identifying a subject in need of treating or preventing a glioma or symptoms thereof; and
administering to the subject a therapeutically effective amount of the immunogenic composition of any one of first arrangements 1-19,
thereby treating or preventing the glioma or symptoms thereof.

52. A method of treating or preventing a large vessel stroke C-IMT or symptoms thereof, comprising:
identifying a subject in need of treating or preventing a large vessel stroke C-IMT or symptoms thereof; and
administering to the subject a therapeutically effective amount of the immunogenic composition of any one of first arrangements 1-19,
thereby treating or preventing the large vessel stroke C-IMT or symptoms thereof.

53. A method of treating or preventing microvascular defects and associated dementias, or symptoms thereof, comprising:
identifying a subject in need of treating or preventing microvascular defects and associated dementias, or symptoms thereof; and
administering to the subject a therapeutically effective amount of the immunogenic composition of any one of first arrangements 1-19,
thereby treating or preventing the microvascular defects and associated dementias, or symptoms thereof.

54. The method of first arrangement 53, wherein the microvascular defects and associated dementias comprises microvascular defects Parkinson's.

55. A method of treating or preventing a peri-implantitis or symptoms thereof, comprising:
identifying a subject in need of treating or preventing a peri-implantitis or symptoms thereof; and
administering to the subject a therapeutically effective amount of the immunogenic composition of any one of first arrangements 1-19,
thereby treating or preventing the peri-implantitis or symptoms thereof.

56. A method of treating or preventing a renal disease or symptoms thereof, comprising:
identifying a subject in need of treating or preventing a renal disease or symptoms thereof; and
administering to the subject a therapeutically effective amount of the immunogenic composition of any one of first arrangements 1-19,
thereby treating or preventing the renal disease or symptoms thereof.

57. A method of treating or preventing a regenerative and stem cell dysfunction, or symptoms thereof, comprising:
identifying a subject in need of treating or preventing a regenerative and stem cell dysfunction, or symptoms thereof; and
administering to the subject a therapeutically effective amount of the immunogenic composition of any one of first arrangements 1-19,
thereby treating or preventing the regenerative and stem cell dysfunction, or symptoms thereof.

58. A method of treating or preventing a condition, disorder or disease associated with a P. gingivalis infection, or symptoms thereof, comprising:
identifying a subject in need of treating or preventing a condition, disorder or disease associated with a P. gingivalis infection, or symptoms thereof; and
administering to the subject a therapeutically effective amount of the immunogenic composition of any one of first arrangements 1-19,
thereby treating or preventing the condition, disorder or disease associated with a P. gingivalis infection, or symptoms thereof.

59. The method of first arrangement 58, comprising administering the therapeutically effective amount of the immunogenic composition to treat the condition, disorder or disease associated with a P. gingivalis infection, or symptoms thereof.

60. The method of first arrangement 58, comprising administering the therapeutically effective amount of the immunogenic composition to prevent the condition, disorder or disease associated with a P. gingivalis infection, or symptoms thereof.

61. The method of any one of first arrangements 58-60, wherein the condition, disorder or disease is associated with a local infection of P. gingivalis.

62. The method of first arrangement 60, wherein the condition, disorder or disease is associated with an oral infection of P. gingivalis.

63. The method of any one of first arrangements 58-62, wherein the condition, disorder or disease is one or more of: vascular disease (e.g., cardiovascular disease, atherosclerosis, coronary artery disease, myocardial infarction, stroke, and myocardial hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD (age-related macular degeneration), cerebral and abdominal aneurysms, glioma, large vessel stroke C-IMT, microvascular defects and associated dementias (e.g., Parkinson's), Peri-Implantitis and/or periodontal disease and/or associated bone loss, cognitive disorders (e.g., early, middle, and/or late dementia; Alzheimer's disease); regenerative and stem cell dysfunction; and longevity or age-related disorder.

64. The method of any one of the preceding first arrangements, wherein the administering comprises administering the immunogenic composition intravenously, subgingivally, intradermally, subcutaneously, intrathecally, or by nebulization.

65. Use of an immunogenic composition of any one of first arrangements 1-19, for treatment of a disorder associated with, caused by or complicated by P. gingivalis.

66. The use of first arrangement 65, wherein the disorder associated with, caused by or complicated by P. gingivalis is one or more of: vascular disease (e.g., cardiovascular disease, atherosclerosis, coronary artery disease, myocardial infarction, stroke, and myocardial hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD (age-related macular degeneration), cerebral and abdominal aneurysms, glioma, large vessel stroke C-IMT, microvascular defects and associated dementias (e.g., Parkinson's), Peri-Implantitis and/or periodontal disease and/or associated bone loss, cognitive disorders (e.g., early, middle, and/or late dementia; Alzheimer's disease); regenerative and stem cell dysfunction; and longevity or age-related disorder.

67. A method of screening for a vaccine, the method comprising:
providing an antibody that binds to a Porphyromonas gingivalis protein PG0495,
determining an epitope to which the antibody binds;
providing a fragment of the Porphyromonas gingivalis protein PG0495 as a protein fragment.

68. The method of first arrangement 67, wherein the epitope is a linear epitope.

69. The method of first arrangement 67, wherein the epitope is a conformational epitope.

70. The method of first arrangement 67, wherein the fragment is provided in a great enough concentration to be therapeutically effective.

71. A prokaryotic or eucaryotic cell, which comprises a recombinant polynucleotide consisting of nucleotides encoding the polypeptide of at least one of:
a polypeptide of YCVEVKYTAGVSPK (SEQ ID NO: 2),
a polypeptide having at least 80% identity to SEQ ID NO: 2,
an immunogenic fragment of 8, 9, 10, 12, 13, or more consecutive amino acids of SEQ ID NO: 2, and
a variant comprising a conservative substitution of at least one amino acid of SEQ ID NO: 2
wherein the polynucleotide is operatively linked to at least one regulatory element.

72. The composition of any one of first arrangements 1-19, further comprising at least one amino acid from HagA and/or gingipiain.

73. The composition of any one of first arrangements 1-19, wherein the protein, epitope, polypeptide, amino acid sequence, or immunogenic fragment comprises at least one additional amino acid from HagA and/or gingipiain.

74. The composition of any of the compositions provided herein, wherein the AP contains a set of one or more paired cysteines.

75. The antigenic composition of method of any one of the preceding first arrangements, wherein the sequence of the peptide is at least 40, 50, 60, 70, 80, 90, 95, 96, 97, 98, 99, or 100% identical to the sequence in FIG. 4 or 5A, 5B, or 5C.

76. The antigenic composition of method of any one of the preceding first arrangements, wherein the sequence of the peptide comprises at least YTYTVYRDGTKIK (SEQ ID NO: 6).

### Second Arrangements

1. An antigenic composition comprising an at least one isolated and purified peptide and/or protein, wherein the isolated and purified peptide and/or protein comprises an epitope that is bound by the antigen binding molecule KB001, wherein the isolated and purified peptide and/or protein is capable of raising an immune response against P. gingivalis in a subject.

2. The antigenic composition of second arrangement 1, wherein the peptide and/or protein comprises a sequence having at least 80% identity to the sequence YTYTVYRDGTKIK (SEQ ID NO: 6).

3. The antigenic composition of any of the above second arrangements, wherein the peptide and/or protein is not complexed with other Arg-specific and Lys-specific P. gingivalis proteinase and adhesin proteins.

4. A composition for use in raising an immune response directed against P. gingivalis in a subject, the composition comprising an effective amount of an isolated and purified peptide and/or protein, wherein the isolated and purified peptide and/or protein comprises an epitope that is bound by the antigen binding molecule KB001, and wherein optionally, the epitope includes at least one of the residues in YTYTVYRDGTKIK (SEQ ID NO: 6).

5. A composition for use in the prevention of a disease or disorder arising from the infection of P. gingivalis in a subject, the composition comprising an effective amount of an at least one isolated and purified peptide and/or protein, wherein the isolated and purified peptide and/or protein comprises an epitope that is bound by the antigen binding molecule KB001, and wherein optionally, the epitope includes at least one of the residues in YTYTVYRDGTKIK (SEQ ID NO: 6).

6. A composition for use as a therapeutic against a disease or disorder arising from the infection of P. gingivalis in a subject, the composition comprising an effective amount of an at least one isolated and purified peptide and/or protein, wherein the isolated and purified peptide and/or protein comprises an epitope that is bound by the antigen binding molecule KB001, and wherein optionally, the epitope includes at least one of the residues in YTYTVYRDGTKIK (SEQ ID NO: 6).

7. The composition of any one of second arrangements 4-6, wherein the peptide and/or protein comprises a sequence having at least 80% identity to the sequence YTYTVYRDGTKIK (SEQ ID NO: 6).

8. The composition of any one of second arrangements 4-7, further comprising a pharmaceutically acceptable carrier.

9. The composition of any one of second arrangements 4-8, further comprising an adjuvant.

10. The composition of second arrangement 9, wherein the adjuvant is an aluminum salt adjuvant.

11. The composition of any one of the above second arrangements , wherein the composition is formulated for use orally.

12. The composition of any one of the above second arrangements, wherein the composition is formulated for percutaneous administration.

13. The composition of any one of the above second arrangements, wherein the subject is mammalian and/or human.

14. The composition of any one of the above second arrangements wherein the peptide and/or protein comprises one, two, three, and/or four sequences having at least 80% identity to one, two, three, and/or all four of the sequences:

(a)
(SEQ ID NO: 1)
GVSPKVCKDVTVEGSNEFAPVQNLT,
(b)
(SEQ ID NO: 2)
YCVEVKYTAGVSPK,
(c)
(SEQ ID NO: 3)
AGTYDFAIAAPQANAKIWIAGQGPTKEDDYVFEAGKKYHFLMKKMGSGD
GTELTISEGGGS
DYTYTVYRDGTKIKEGLTATTFEEDGVAAGNHEYCVEVKYTAGVSPKVC
KDVTVEGSNEFAPVQNLT,
and/or
(d)
(SEQ ID NO: 4)
GVSPK.

15. The composition of any one of the above second arrangements, wherein the epitope is a conformational epitope defined by the sequence YCVEVKYTAGVSPK or YTYTVYRDGTKIK (SEQ ID NO: 6).

16. The composition of any one of the above second arrangements, wherein the epitope is a conformational epitope defined by the sequence YTYTVYRDGTKIK (SEQ ID NO: 6), wherein a structure of the confirmational epitope is effectively the same as a structure of YTYTVYRDGTKIK (SEQ ID NO: 6) within a sequence of Kgp, wherein 1, 2, 3, 4, 5, 6, 7, 8, 9, 10, 11, 12, or all 13 of the residues in the sequence are present as part of the epitope.

17. The composition of any one of the above second arrangements, wherein the epitope is a conformational epitope defined by the sequence AGTYDFAIAAPQANAKIWIAGQGPTKEDDYVFEAGKKYHFLMKKMGSGDGTELTIS EGGGSDYTYTVYRDGTKIKEGLTATTFEEDGVAAGNHEYCVEVKYTAGVSPKVCK DVTVEGSNEFAPVQNLT (SEQ ID NO: 3), wherein a structure of the confirmational epitope is effectively the same as a structure of AGTYDFAIAAPQANAKIWIAGQGPTKEDDYVFEAGKKYHFLMKKMGS GDGTELTIS EGGGSDYTYTVYRDGTKIKEGLTATTFEEDGVAAGNHEYCVEVKYTAGVSPKVCK DVTVEGSNEFAPVQNLT (SEQ ID NO: 3).

18. The composition of any one of the above second arrangements, wherein the peptide is no longer than 20 amino acids in length.

19. The composition of any one of the above second arrangements, wherein the peptide is no longer than 20 amino acids in length.

20. The composition of any one of the above second arrangements, wherein the peptide is no longer than 14 amino acids in length.

21. A method of treating a disease or condition associated with the presence of P. gingivalis in an oral tissue of a subject, comprising administering to a subject a composition of any one of the previous second arrangements and administering to the subject a peptide, wherein the polypeptide comprises: YCVEVKYTAGVSPK or YTYTVYRDGTKIK (SEQ ID NO: 6), a polypeptide having at least 80% identity to this sequence, an immunogenic fragment of 8, 9, 10, 12, 13, or more consecutive amino acids of this sequence, and a variant comprising a conservative substitution of at least one amino acid of this sequence.

22. The method of second arrangement 21, wherein the immunogenic composition is administered at least two times.

23. A method of treating or preventing a vascular disease or symptoms thereof, comprising:
identifying a subject in need of treating or preventing a vascular disease or symptoms thereof; and
administering to the subject a therapeutically effective amount of the immunogenic composition any one of second arrangements 1-20,
thereby treating or preventing the vascular disease or symptoms thereof.

24. The method of second arrangement 23, wherein the vascular disease comprises cardiovascular disease, atherosclerosis, coronary artery disease, myocardial infarction, stroke, and myocardial hypertrophy.

25. The method of second arrangement 23 or 24, further comprising administering to the subject at least one other therapeutic agent for treating or preventing the vascular disease, or symptoms thereof.

26. The method of second arrangement 25, wherein the other therapeutic agent comprises a serum lipid lowering agent.

27. The method of second arrangement 26, wherein the other therapeutic agent is a statin.

28. A method of treating or preventing a vascular disease or symptoms thereof, comprising:
administering to a subject in need of treating or preventing a vascular disease, or symptoms thereof, a therapeutically effective amount of at least one therapeutic agent for treating or preventing the vascular disease, or symptoms thereof; and
administering an effective amount of the immunogenic composition of any one of second arrangements 1-20, to thereby enhance the therapeutic effect of the at least one therapeutic agent.

29. The method of second arrangement 28, wherein the other therapeutic agent comprises a serum lipid lowering agent.

30. The method of second arrangement 29, wherein the other therapeutic agent is a statin.

31. A method of treating or preventing a systemic disease or symptoms thereof, comprising:
identifying a subject in need of treating or preventing a systemic disease or symptoms thereof, wherein the systemic disease is one or more of type II diabetes, insulin resistance and metabolic syndrome; and
administering to the subject a therapeutically effective amount of the immunogenic composition of any one of second arrangements 1-20,
thereby treating or preventing the systemic disease or symptoms thereof.

32. A method of treating or preventing rheumatoid arthritis or symptoms thereof, comprising:
identifying a subject in need of treating rheumatoid arthritis or symptoms thereof; and
administering to the subject a therapeutically effective amount of the immunogenic composition of any one of second arrangements 1-20,
thereby treating or preventing the rheumatoid arthritis or symptoms thereof.

33. A method of treating or preventing cancer or symptoms thereof, comprising:
identifying a subject in need of treating cancer or symptoms thereof; and
administering to the subject a therapeutically effective amount of the immunogenic composition of any one of second arrangements 1-20,
thereby treating or preventing the cancer or symptoms thereof.

34. The method of second arrangement 33, wherein the cancer is oral, gastrointestinal, lung or pancreatic cancer.

35. The method of second arrangement 33 or 34, further comprising administering to the subject at least one other therapeutic agent for treating or preventing the cancer, or symptoms thereof.

36. The method of second arrangement 35, wherein the other therapeutic agent comprises a small molecule drug or immunotherapeutic agent.

37. A method of treating or preventing cancer or symptoms thereof, comprising:
administering to a subject in need of treating or preventing cancer, or symptoms thereof, a therapeutically effective amount of at least one therapeutic agent for treating or preventing the cancer, or symptoms thereof; and
administering an effective amount of the immunogenic composition of any one of second arrangements 1-20, to thereby enhance the therapeutic effect of the at least one therapeutic agent.

38. The method of second arrangement 37, wherein the at least one therapeutic agent comprises a small molecule drug or immunotherapeutic agent.

39. The method of second arrangement 37 or 38, wherein the cancer is oral, gastrointestinal, lung or pancreatic cancer.

40. A method of treating or preventing a gut microbiome-related disorder or symptoms thereof, comprising:
identifying a subject in need of treating a gut microbiome-related disorder or symptoms thereof; and
administering to the subject a therapeutically effective amount of the immunogenic composition of any one of second arrangements 1-20,
thereby treating or preventing the gut microbiome-related disorder or symptoms thereof.

41. The method of second arrangement 40, wherein the gut microbiome-related disorder comprises inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity.

42. A method of treating or preventing a cognitive disorder or symptoms thereof, comprising:
identifying a subject in need of treating a cognitive disorder or symptoms thereof; and
administering to the subject a therapeutically effective amount of the immunogenic composition of any one of second arrangements 1-20,
thereby treating or preventing the cognitive disorder or symptoms thereof.

43. The method of second arrangement 42, wherein the cognitive disorder is Alzheimer's disease.

44. The method of second arrangement 42 or 43, wherein the cognitive disorder is early, middle or late dementia.

45. A method of treating or preventing an age-related or longevity-related disorder, or symptoms thereof, comprising:
identifying a subject in need of treating an age-related or longevity-related disorder; and
administering to the subject a therapeutically effective amount of the immunogenic composition of any one of second arrangements 1-20,
thereby treating or preventing the age-related or longevity-related disorder, or symptoms thereof.

46. A method of treating or preventing a post event myocardial hypertrophy or symptoms thereof, comprising:
identifying a subject in need of treating or preventing a post event myocardial hypertrophy or symptoms thereof; and
administering to the subject a therapeutically effective amount of the immunogenic composition of any one of second arrangements 1-20,
thereby treating or preventing the post event myocardial hypertrophy or symptoms thereof.

47. A method of treating a wound, comprising:
identifying a subject in need of treating a wound; and
administering to the subject a therapeutically effective amount of the immunogenic composition of any one of second arrangements 1-20,
whereby closure of the wound is enhanced, thereby treating the wound.

48. A method of treating or preventing an age-related macular degeneration (AMD) or symptoms thereof, comprising:
identifying a subject in need of treating or preventing AMD or symptoms thereof; and
administering to the subject a therapeutically effective amount of the immunogenic composition of any one of second arrangements 1-20,
thereby treating or preventing the AMD or symptoms thereof.

49. A method of treating or preventing an aneurysm or symptoms thereof, comprising:
identifying a subject in need of treating or preventing an aneurysm or symptoms thereof; and
administering to the subject a therapeutically effective amount of the immunogenic composition of any one of second arrangements 1-20,
thereby treating or preventing the aneurysm or symptoms thereof.

50. The method of second arrangement 49, wherein the aneurysm is a cerebral or abdominal aneurysm.

51. A method of treating or preventing a glioma or symptoms thereof, comprising:
identifying a subject in need of treating or preventing a glioma or symptoms thereof; and
administering to the subject a therapeutically effective amount of the immunogenic composition of any one of second arrangements 1-20,
thereby treating or preventing the glioma or symptoms thereof.

52. A method of treating or preventing a large vessel stroke C-IMT or symptoms thereof, comprising:
identifying a subject in need of treating or preventing a large vessel stroke C-IMT or symptoms thereof; and
administering to the subject a therapeutically effective amount of the immunogenic composition of any one of second arrangements 1-20,
thereby treating or preventing the large vessel stroke C-IMT or symptoms thereof.

53. A method of treating or preventing microvascular defects and associated dementias, or symptoms thereof, comprising:
identifying a subject in need of treating or preventing microvascular defects and associated dementias, or symptoms thereof; and
administering to the subject a therapeutically effective amount of the immunogenic composition of any one of second arrangements 1-20,
thereby treating or preventing the microvascular defects and associated dementias, or symptoms thereof.

54. The method of second arrangement 53, wherein the microvascular defects and associated dementias comprises microvascular defects Parkinson's.

55. A method of treating or preventing a peri-implantitis or symptoms thereof, comprising:
identifying a subject in need of treating or preventing a peri-implantitis or symptoms thereof; and
administering to the subject a therapeutically effective amount of the immunogenic composition of any one of second arrangements 1-20,
thereby treating or preventing the peri-implantitis or symptoms thereof.

56. A method of treating or preventing a renal disease or symptoms thereof, comprising:
identifying a subject in need of treating or preventing a renal disease or symptoms thereof; and
administering to the subject a therapeutically effective amount of the immunogenic composition of any one of second arrangements 1-20,
thereby treating or preventing the renal disease or symptoms thereof.

57. A method of treating or preventing a regenerative and stem cell dysfunction, or symptoms thereof, comprising:
identifying a subject in need of treating or preventing a regenerative and stem cell dysfunction, or symptoms thereof; and
administering to the subject a therapeutically effective amount of the immunogenic composition of any one of second arrangements 1-20,
thereby treating or preventing the regenerative and stem cell dysfunction, or symptoms thereof.

58. A method of treating or preventing a condition, disorder or disease associated with a P. gingivalis infection, or symptoms thereof, comprising:
identifying a subject in need of treating or preventing a condition, disorder or disease associated with a P. gingivalis infection, or symptoms thereof; and
administering to the subject a therapeutically effective amount of the immunogenic composition of any one of second arrangements 1-20,
thereby treating or preventing the condition, disorder or disease associated with a P. gingivalis infection, or symptoms thereof.

59. The method of second arrangement 58, comprising administering the therapeutically effective amount of the immunogenic composition to treat the condition, disorder or disease associated with a P. gingivalis infection, or symptoms thereof.

60. The method of second arrangement 58, comprising administering the therapeutically effective amount of the immunogenic composition to prevent the condition, disorder or disease associated with a P. gingivalis infection, or symptoms thereof.

61. The method of any one of second arrangements 58-60, wherein the condition, disorder or disease is associated with a local infection of P. gingivalis.

62. The method of second arrangement 60, wherein the condition, disorder or disease is associated with an oral infection of P. gingivalis.

63. The method of any one of second arrangements 58-62, wherein the condition, disorder or disease is one or more of: vascular disease (e.g., cardiovascular disease, atherosclerosis, coronary artery disease, myocardial infarction, stroke, and myocardial hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD (age-related macular degeneration), cerebral and abdominal aneurysms, glioma, large vessel stroke C-IMT, microvascular defects and associated dementias (e.g., Parkinson's), Peri-Implantitis and/or periodontal disease and/or associated bone loss, cognitive disorders (e.g., early, middle, and/or late dementia; Alzheimer's disease); regenerative and stem cell dysfunction; and longevity or age-related disorder.

64. The method of any one of the preceding second arrangements, wherein the administering comprises administering the immunogenic composition intravenously, subgingivally, intradermally, subcutaneously, intrathecally, or by nebulization.

65. Use of an immunogenic composition of any one of second arrangements 1-20, for treatment of a disorder associated with, caused by or complicated by P. gingivalis.

66. The use of second arrangement 65, wherein the disorder associated with, caused by or complicated by P. gingivalis is one or more of: vascular disease (e.g., cardiovascular disease, atherosclerosis, coronary artery disease, myocardial infarction, stroke, and myocardial hypertrophy); systemic disease (e.g., type II diabetes, insulin resistance and metabolic syndrome); rheumatoid arthritis; cancer (e.g., oral, gastrointestinal, or pancreatic cancer); renal disease, gut microbiome-related disorder (e.g., inflammatory bowel disease, irritable bowel syndrome (IBS), coeliac disease, non-alcoholic fatty liver disease (NAFLD), non-alcoholic steatohepatitis (NASH), allergy, asthma, metabolic syndrome, cardiovascular disease, and obesity); post event myocardial hypertrophy, wound closure, AMD (age-related macular degeneration), cerebral and abdominal aneurysms, glioma, large vessel stroke C-IMT, microvascular defects and associated dementias (e.g., Parkinson's), Peri-Implantitis and/or periodontal disease and/or associated bone loss, cognitive disorders (e.g., early, middle, and/or late dementia; Alzheimer's disease); regenerative and stem cell dysfunction; and longevity or age-related disorder.

67. A method of screening for a vaccine, the method comprising:
providing an antibody that binds to a Porphyromonas gingivalis protein PG0495,
determining an epitope to which the antibody binds; and
providing a fragment of the Porphyromonas gingivalis protein PG0495 as a protein fragment.

68. The method of second arrangement 67, wherein the epitope is a linear epitope.

69. The method of second arrangement 67, wherein the epitope is a conformational epitope.

70. The method of second arrangement 67, wherein the fragment is provided in a great enough concentration to be therapeutically effective.

71. A prokaryotic or eucaryotic cell, which comprises a recombinant polynucleotide consisting of nucleotides encoding the polypeptide of at least one of:
a polypeptide of YCVEVKYTAGVSPK or YTYTVYRDGTKIK (SEQ ID NO: 6),
a polypeptide having at least 80% identity to SEQ ID NO: 6,
an immunogenic fragment of 8, 9, 10, 12, 13, or more consecutive amino acids of SEQ ID NO: 6, and
a variant comprising a conservative substitution of at least one amino acid of SEQ ID NO: 6,
wherein the polynucleotide is operatively linked to at least one regulatory element.

72. The composition of any one of second arrangements 1-20, further comprising at least one amino acid from HagA and/or gingipiain.

73. The composition of any one of second arrangements 1-20, wherein the protein, epitope, polypeptide, amino acid sequence, or immunogenic fragment comprises at least one additional amino acid from HagA and/or gingipiain.

74. The composition of any of the compositions provided herein, wherein the AP contains a set of one or more paired cysteines.

75. The antigenic composition of method of any one of the preceding second arrangements, wherein the sequence of the peptide is at least 40, 50, 60, 70, 80, 90, 95, 96, 97, 98, 99, or 100% identical to the sequence in FIG. 4 or 5A, 5B, or 5C.

76. The antigenic composition of method of any one of the preceding second arrangements, wherein the sequence of the peptide comprises at least YTYTVYRDGTKIK (SEQ ID NO: 6).

77. An isolated peptide or polypeptide comprising:
at least four or more amino acids from YTYTVYRDGTKIK (SEQ ID NO: 6).

78. The isolated peptide or polypeptide of second arrangement 77, wherein the isolated peptide or polypeptide is present in a therapeutically effective amount in a vaccine.

79. The isolated peptide or polypeptide of second arrangement 77 or 78, wherein at least 5, 6, 7, 8, 9, 10, 11, 12, or 13 of the amino acids are present in the polypeptide.

80. The isolated peptide or polypeptide of any one of second arrangements 77-79,
wherein the sequence terminates at the n-terminal end, the c-terminal end, or both the n-terminal and the c-terminal end.

81. The isolated peptide or polypeptide of any one of second arrangements 77-80, wherein the sequence consists essentially of YTYTVYRDGTKIK (SEQ ID NO: 6).

82. The method or composition or isolated protein, peptide, or polypeptide of any one of the preceding second arrangements, wherein:
a) the protein, polypeptide, or peptide further comprises, consists, or consists essentially of a combination of a GST-TEV-gingipain-His as a fusion protein (e.g., SEQ ID Nos. 85-87), or a variant thereof that is at least 70, 75, 80, 85, 90, 95, 96, 97, 98, 99, or more percent identical or similar to SEQ ID Nos. 85-87 fused together;
b) the protein, polypeptide, or peptide further comprises, consists, or consists essentially of a combination of GST -gingipain as a fusion protein (e.g., SEQ ID No: 90), or a variant thereof that is at least 70, 75, 80, 85, 90, 95, 96, 97, 98, 99, or more percent identical or similar to SEQ ID Nos. 90;
c) the protein, polypeptide, or peptide further comprises, consists, or consists essentially of a combination of a KGP-RGP chimeric (e.g., SEQ ID Nos. 88-89), or a variant thereof that is at least 70, 75, 80, 85, 90, 95, 96, 97, 98, 99, or more percent identical or similar to SEQ ID Nos. 88-89 fused together; or
d) the protein, polypeptide, or peptide further comprises, consists, or consists essentially of a combination of a HagA 3x recombinant epitope (e.g., SEQ ID No: 84), or a variant thereof that is at least 70, 75, 80, 85, 90, 95, 96, 97, 98, 99, or more percent identical or similar to SEQ ID Nos. 84.

83. An amino acid sequence comprising:
a) at least 1, 2, 3, 4, 5, 6, 7, 8, 9, 10, 11, 12, or all of the amino acids at any one or more of positions 1, 2, 3, 4, 5, 6, 7, 8, 9, 10, 11, 12, and/or 13 of YTYTVYRDGTKIK (SEQ ID NO: 6) (where the first Y is position 1 and the last K is position 13); and
b) at least one of:
   i) the amino acid sequence of SEQ ID Nos. 85-87, or a variant thereof that is at least 50, 60, 70, 75, 80, 85, 90, 95, 96, 97, 98, 99, or more percent identical or similar to SEQ ID Nos. 85-87 fused together,
   ii) the amino acid sequence of SEQ ID No: 90, or a variant thereof that is at least 50, 60, 70, 75, 80, 85, 90, 95, 96, 97, 98, 99, or more percent identical or similar to SEQ ID Nos. 90,
   iii) the amino acid sequence of SEQ ID Nos. 88-89), or a variant thereof that is at least 50, 60, 70, 75, 80, 85, 90, 95, 96, 97, 98, 99, or more percent identical or similar to SEQ ID Nos. 88-89 fused together, or
   iv) the amino acid sequence of SEQ ID No: 84, or a variant thereof that is at least 50, 60, 70, 75, 80, 85, 90, 95, 96, 97, 98, 99, or more percent identical or similar to SEQ ID Nos. 84,
   wherein a) can be fused to b) or wherein a) can be a subsequence within b).

84. Any of the compositions, uses, or methods provided above, wherein the amino acid sequence of second arrangement 83 is used instead of the otherwise denoted AP, amino acid sequence, peptide, protein, or polypeptide in any one of second arrangements 1-82.

### EXAMPLES

The technology described herein is further illustrated by the following examples which in no way should be construed as being further limiting.

### Example 1

### Epitope Mapping of KB-001

This non-limiting example shows a procedure for tryptic digest and mass spectrometry (MS) analysis of gingipains for epitope mapping of KB-001. Such epitopes can be used to define various APs.

To determine viable APs, one can first identify the epitope on P. gingivalis target proteins of KB-001, gingipains (RgpA, Kgp) and hemagglutinin from various P. gingivalis strains were digested with trypsin and the tryptic digests were probed for KB-001 binding (FIG. 1). Peptides fragments binding to KB-001 were analyzed by MS and N-terminal sequencing.

The deduced sequences of linear portion KB -001-binding fragments and the position of these sequences in the full protein are listed in FIGS. 2A-2J. Linear analysis indicated that the binding epitope included: YCVEVKYTAGVSPK (SEQ ID NO: 2). Thus, a viable AP would include, in some embodiments, this sequence.

Sequences within gingipains (RgpA, Kgp) and hemagglutinin (HagA) from various P. gingivalis strains that encompass the putative linear epitope sequence recognized by KB-001 are indicated in FIGS. 3A-3F. HagA from W83 and ATCC33277 contain 3 and 4 nearly perfect repeats, respectively, of the sequence containing the putative epitope (FIGS. 3C, 3D, 3E, 3F). As a nearly perfect the motif occurs twice in gingipain structure (FIGS. 3D, 3E, 3F). The third repeat is present in HA4 domain of RgpA but is degenerate in the Kgp (from W83 strain). The presence of the epitope within the sequences show in FIG. 3F was verified by WB analysis of mAbs reactivity with different domains of RgpA and Kgp.

In some embodiments, an AP of the present disclosure includes any one or more of the following sequences:

1.
(SEQ ID NO: 25)
PASYTYTVYRDGTKIKEGLTATTFEEDGVAAGNHEYCVEVKYTA
GVSPKVC;
2.
(SEQ ID NO: 26)
GSDYTYTVYRDGTKIKEGLTATTFEEDGVATGNHEYCVEVKYTA
GVSPKVC;
3.
(SEQ ID NO: 27)
PTDYTYTVYRDGTKIKEGLTETTFEEDGVATGNHEYCVEVKYTAG
VSPKKC;
4.
(SEQ ID NO: 28)
PTDYTYTVYRDGTKIKEGLTETTFEEDGVATGNHEYCVEVKYTAG
VSPKEC;
5.
(SEQ ID NO: 29)
PTDYTYTVYRDGTKIKEGLTETTFEEDGVATGNHEYCVEVKYTAG
VSPKVC;
6.
(SEQ ID NO: 30)
PASYTYTVYRDGTKIKEGLTETTYRDAGMSAQSHEYCVEVKYTA
GVSPKVC;
and/or
7.
(SEQ ID NO: 31)
APSYTYTIYRNNTQIASGVTETTYRDPDLATGFYTYGVKVVYPNG
ESAIET.

### Example 2

### Distribution of “XXX Epitope” Domains in Autopsy Tissues from AD Brains

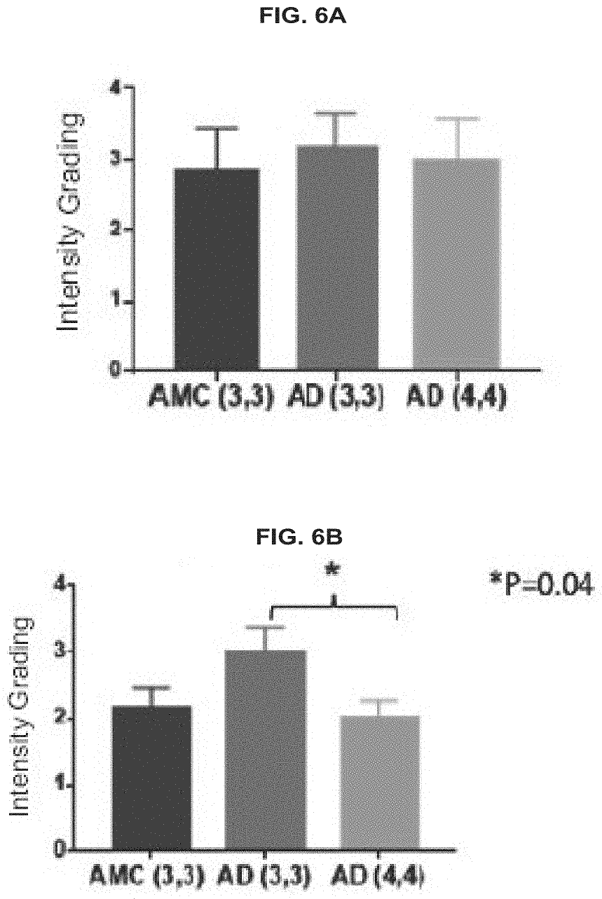

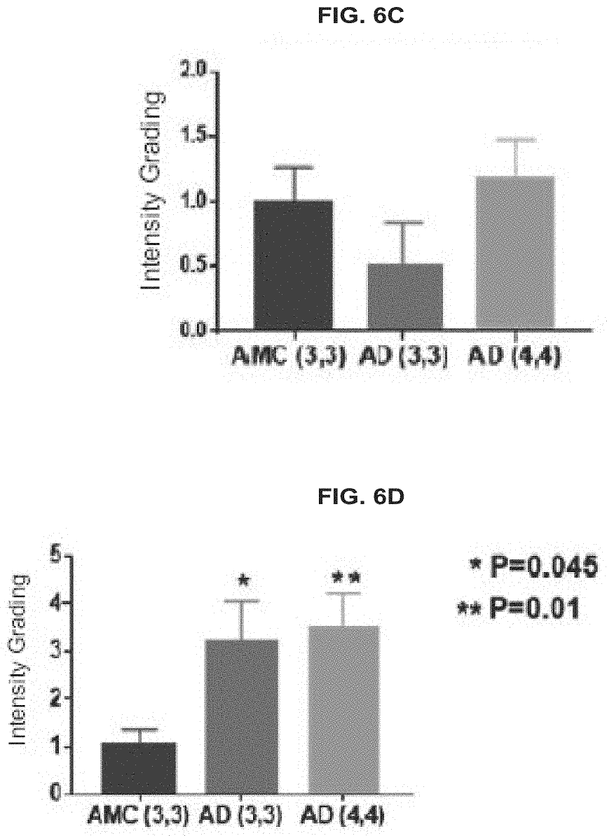

As disclosed herein, the inventors investigated the distribution of the XXX Epitope in AD Brains. Three subjects were examined: the control patient (AMC 3,3), patient #1 with Alzheimer's Disease (AD3,3), and patient #2 with Alzheimer's Disease (AD4,4). Autopsy tissues from each of the patients were collected, and stained using a novel antibody that binds to the XXX Epitope of gingipain. The staining was quantified and compared between the three patients to determine the quantity of gingipain in the front lobe, occipital lobe, cerebellum, and hippocampus (FIGS. 6A-6D). The densities of immunohistochemical intensity of P. gingipains were assessed relative to none (0) on a scale of 1 to 5 in 7μ sections of temporal lobe/hippocampal area from brains of the age matched control (“AMC”) who were clinically and neuropathologically evaluated by Braak and Braak, and by antibody staging of appropriate region analysis (see Table 1 below). Similar assessments were made of analogous areas from brains of patients that were evaluated and determined to be neuropathologically as having met the criteria for a diagnosis of Alzheimer's disease.

TABLE 1
Densitometric comparisons of P. gingipains in Alzheimer
and control brains, segregated by APOE genotypes 3,3 or 4,4

| | | |
|---|---|---|
| AMC 3,3- | AD 3,3- | Diagnosis |

| | | | | | |
|---|---|---|---|---|---|
| G03-26 | 1 | C03-54 | 1 | C0029 | 5 |
| G04-05 | 2 | C05-51 | 4 | C01-80 | 5 |
| G04-21 | 2 | C05-64 | 0 | C07-71 | 5 |
| G05-17 | 0 | C06-35 | 5 | C99-76 | 1 |
| G97-86 | 1 | G04-15 | 5 | G01-78 | 3 |
| G98-114 | 0 | G90-122 | 4 | G03-16 | 2 |

Surprisingly, no significant difference was detected from gingipain antibody staining in the frontal lobe region between control and AD patients. In contrast, AD patient had significantly higher gingipain antibody signal intensity in the hippocampus region. To verify this finding, forty-six brain tissue samples were collected from frontal and temporal biopsies of 23 brains specimens. Of these specimens, 16 were age matched controls and 7 were AD patients. The tissue samples were subjected to a PCR-based liquid hybridization assay (PCR-LH) to detect P. gingivalis DNA. All samples were negative for P. gingivalis DNA (see Table 2 below).

TABLE 2
PCR-LH screening for P. gingivalis DNA in brain tissue samples

| | | diag- | | P. gingivalis | IHC |
|---|---|---|---|---|---|
| sample_id | specimen_id | nosis | location | detection | result* |
| uams_S21-1 | uams_S21 | AD | frontal | Negative | |
| uams_S21-2 | uams_S21 | AD | temporal | Negative | |
| uams_S7-3 | uams_S7 | AD | frontal | Negative | |
| uams_S7-4 | uams_S7 | AD | temporal | Negative | |
| uams_S6-5 | uams_S6 | AD | frontal | Negative | |
| uams_S6-6 | uams_S6 | AD | temporal | Negative | |
| uams_S9-7 | uams_S9 | AD | frontal | Negative | |
| uams_S9-8 | uams_S9 | AD | temporal | Negative | |
| uams_S3-9 | uams_S3 | AD | frontal | Negative | |
| uams_S3-10 | uams_S3 | AD | temporal | Negative | |
| uams_S32-11 | uams_S32 | AD | frontal | Negative | |
| uams_S32-12 | uams_S32 | AD | temporal | Negative | |
| uams_S29-13 | uams_S29 | AD | frontal | Negative | |
| uams_S29-14 | uams_S29 | AD | temporal | Negative | |
| uams_S26-49 | uams_S26 | AMC | frontal | Negative | Positive |
| uams_S26-50 | uams_S26 | AMC | temporal | Negative | Positive |
| uams_S23-51 | uams_S23 | AMC | frontal | Negative | |
| uams_S23-52 | uams_S23 | AMC | temporal | Negative | |
| uams_S12-53 | uams_S12 | AMC | frontal | Negative | |
| uams_S12-54 | uams_S12 | AMC | temporal | Negative | |
| uams_S1-55 | uams_S1 | AMC | frontal | Negative | |
| uams_S1-56 | uams_S1 | AMC | temporal | Negative | |
| uams_S22-57 | uams_S22 | AMC | frontal | Negative | |
| uams_S22-58 | uams_S22 | AMC | temporal | Negative | |
| uams_S14-59 | uams_S14 | AMC | frontal | Negative | Positive |
| uams_S14-60 | uams_S14 | AMC | temporal | Negative | Positive |
| uams_S28-61 | uams_S28 | AMC | frontal | Negative | |
| uams_S28-62 | uams_S28 | AMC | temporal | Negative | |
| uams_S30-63 | uams_S30 | AMC | frontal | Negative | |
| uams_S30-64 | uams_S30 | AMC | temporal | Negative | |
| uams_S13-33 | uams_S13 | AMC | frontal | Negative | |
| uams_S13-34 | uams_S13 | AMC | temporal | Negative | |
| uams_S5-35 | uams_S5 | AMC | frontal | Negative | |
| uams_S5-36 | uams_S5 | AMC | temporal | Negative | |
| uams_S10-37 | uams_S10 | AMC | frontal | Negative | Positive |
| uams_S10-38 | uams_S10 | AMC | temporal | Negative | Positive |
| uams_S11-39 | uams_S11 | AMC | frontal | Negative | |
| uams_S11-40 | uams_S11 | AMC | temporal | Negative | |
| uams_S18-41 | uams_S18 | AMC | frontal | Negative | |
| uams_S18-42 | uams_S18 | AMC | temporal | Negative | |
| uams_S16-43 | uams_S16 | AMC | frontal | Negative | |
| uams_S16-44 | uams_S16 | AMC | temporal | Negative | |
| uams_S19-45 | uams_S19 | AMC | frontal | Negative | |
| uams_S19-46 | uams_S19 | AMC | temporal | Negative | |
| uams_S20-47 | uams_S20 | AMC | frontal | Negative | |
| uams_S20-48 | uams_S20 | AMC | temporal | Negative | |

### Example 3

### “XXX Epitope” in AD Tissue Detected Using KB-001

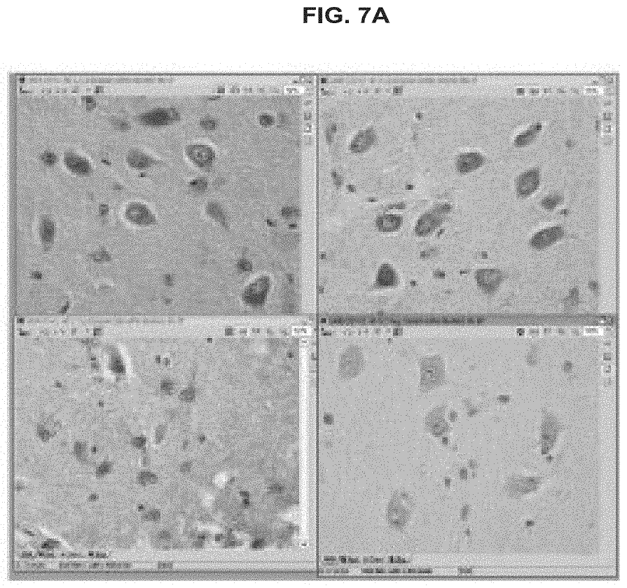

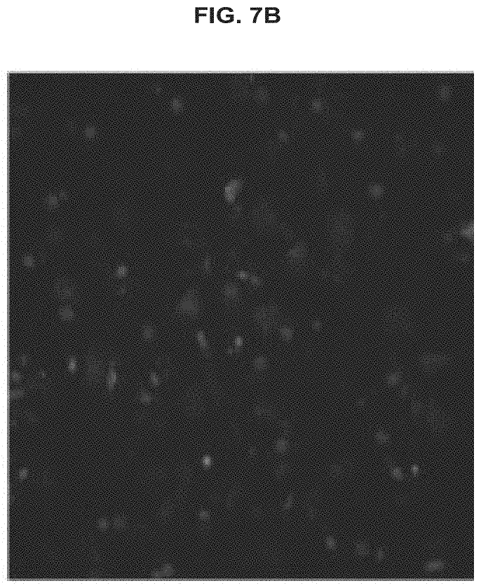

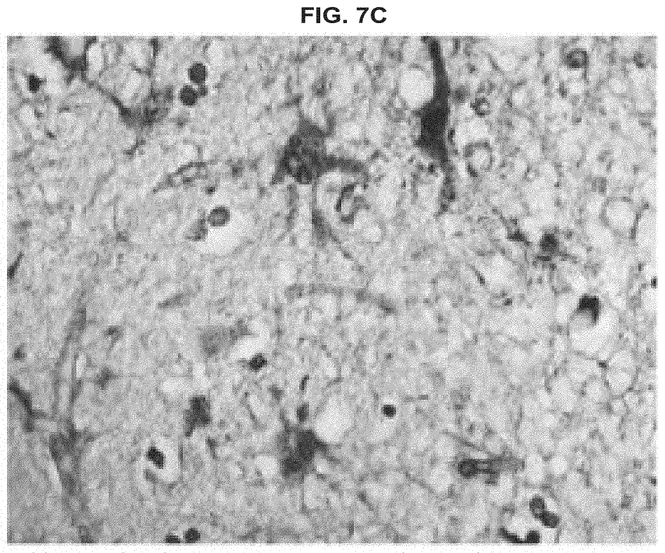

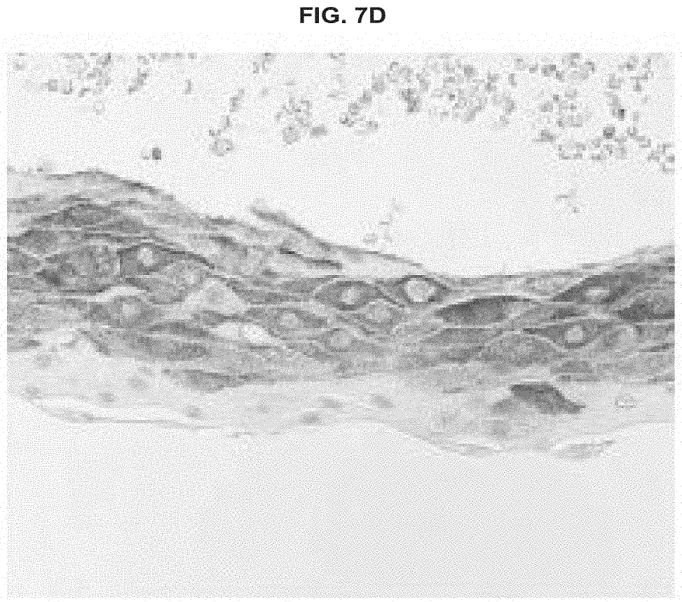

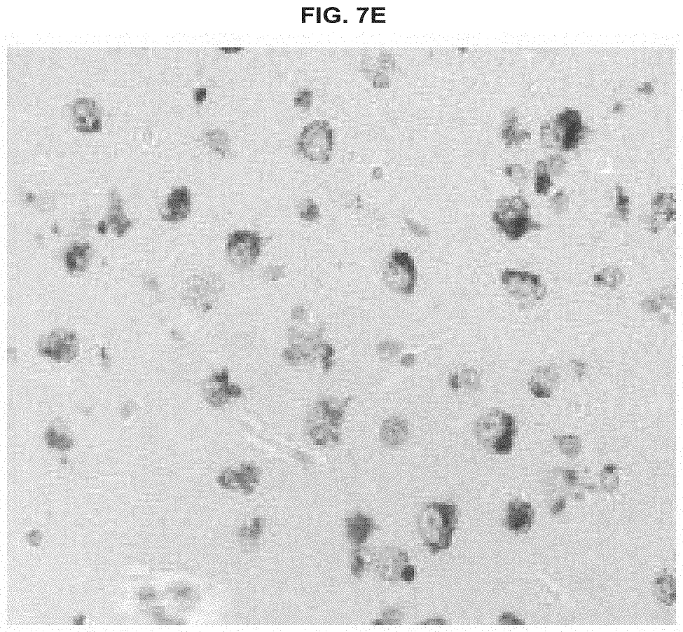

As disclosed herein, KB-001 was used to detect gingipain in AD tissues. Tissues were collected from AD patients, and gingipain was labeled for imaging using KB-001 in brain tissue, gingival tissue, the frontal lobe, and the choroid plexus (FIGS. 7A-7F).

Periodontal disease has been implicated as a risk factor for Alzheimer's disease (AD). Neuropathological characteristics of AD includes accumulation of amyloid-beta (Aβ), which may be related to an innate immune response to infection. To test the hypothesis that periodontal P. gingivalis infection can induce immune responses in the brain, a brain tissue section from a deceased AD patient was immunohistochemically assayed using KB001.

Immunohistochemistry was performed for the detection of Pg/gingipain on formalin fixed paraffin embedded human brain sections. Seven micron sections were de-paraffinized using xylenes followed by rehydration through a series of decreasing percentages of ethanol, ending in dH2O. Antigen retrival was performed using Tris EDTA buffer (pH8) in a 100 degree waterbath for 30 minutes followed by 30 minutes of benchtop cooling. Tissue permeablization was done in the PBS buffer rinses containing 0.1% of Tween 20. Endogenous peroxidase was quenched using a 3% solution of hydrogen peroxide in methanol. Blocking with 5% normal goat serum for 1 hr. was applied just prior to application of primary antibody. Pg/gingipain antibody is used at 1:100 diluted in 5% normal goat serum. Sections were incubated overnight at 4° C. Secondary antibody used was Goat anti Mouse ImmPRESS reagent (Vector MP-7452) diluted 1:1 in 5% normal goat serum for 30 minutes. Visualization was achieved using ImmPACT DAB (Vector SK-4105) for 3-5 minutes. Nuclear staining was with Mayer's Hematoxylin (Abcam ab220365). Negative control was brain tissue section reacted without the application of the Gingipain antibody. Positive control used was gingival tissue.

FIG. 7C shows a representative image of staining of the tissue section by KB001. The granular staining was observed in hippocampal neurons, microglia and astrocytes, as the antibody bound to gingipain or other P. gingivalis-derived targets in the cells. Thus, KB001 appeared to bind directly to the accumulated exo-toxins in the brain of the AD patient. The antibody labeled neurons, astrocytes and micro-glial cells. FIG. 7A shows further staining of brain tissue sections from an AD patient, using KB001. The staining indicates binding of KB001 to intra-cellular accumulated gingipains located in the brain. FIG. 7E shows IHC staining of the frontal lobe using KB001. These results indicate accumulation of P. gingivalis exo-toxins can occur in an AD patient's brain.

IHC of 18 hippocampal sections were evaluated and 10 of these were found to be positive (FIG. 7F). As a positive control, KB001 was used to stain gum tissue from a biopsy of a P. gingivalis colonized patient. The granules are the intra-cellular cytoplasmic localized gingipains as detected with KB001 (FIG. 7D).

### Example 4

### KB-001 Binding Analysis

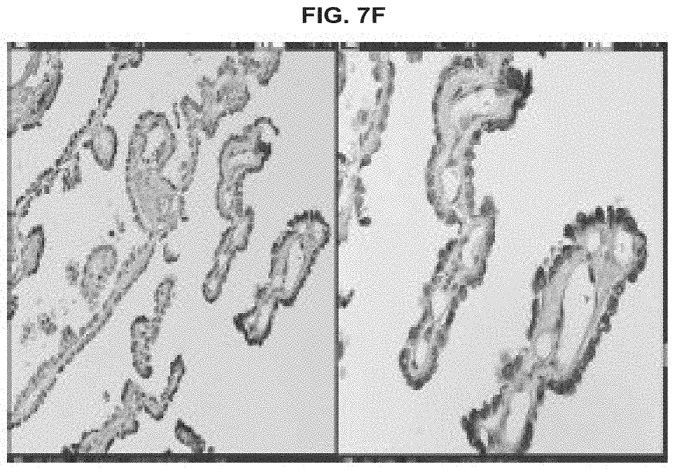

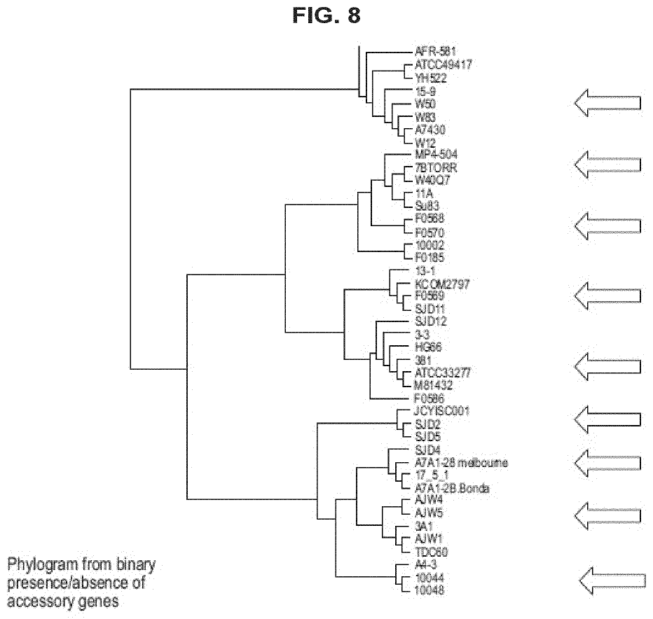

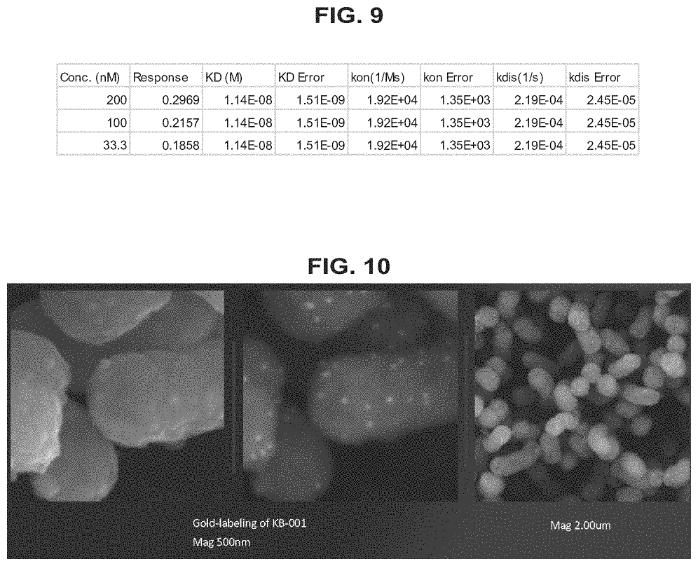

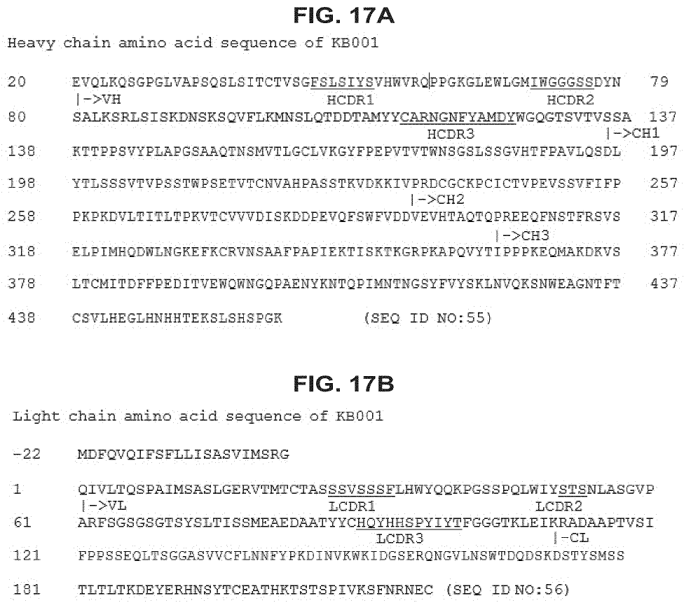

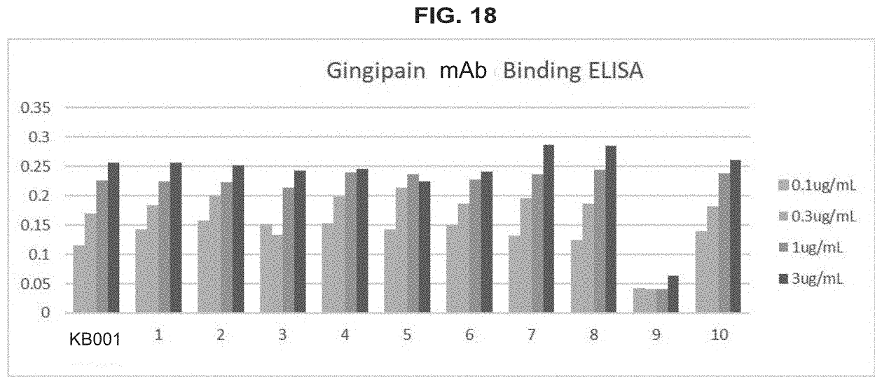

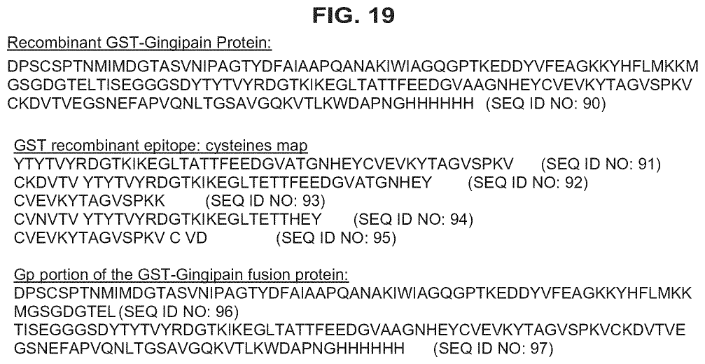

As disclosed herein, the binding profile of KB-001 (the amino acid sequence of which is shown in FIGS. 17A and 17B) was assessed. Clinical isolates as well as pathologically significant strains of P.g. were genetically characterized to identify the phylogenetic diversity. Immuno-electron microscopy of genetically diverse strains of P.g. was done by immunogold labeling to detect specificity of KB-001 against P.g (FIG. 10). Ten strains that represent the diversity of strains as determined by comparison of genome sequences were chosen for analysis (FIG. 8). The KB-001 antibody was found to bind all genetically diverse strains representing the entire P.g. family. In further analysis, KB-001 recognized all 22 laboratory strains and serotypes of P.g. tested as well as 105 human clinical isolates (data not shown). The binding affinity of KB-001 to whole bacterial cells of P.g. was then assessed (Table 4 and FIG. 9). KB-001 demonstrated high affinity for P.g. with a nanomolar binding affinity.

TABLE 4
Binding affinity of KB-001 to whole bacterial cells of P. gingivalis

| Conc. (nM) | Response | KD (M) | KD Error |
|---|---|---|---|
| 200 | 0.2969 | 1.14E−08 | 1.51E−09 |
| 100 | 0.2157 | 1 14E−08 | 1.51E−09 |
| 33.3 | 0.1858 | 1.14E−08 | 1.51E−09 |

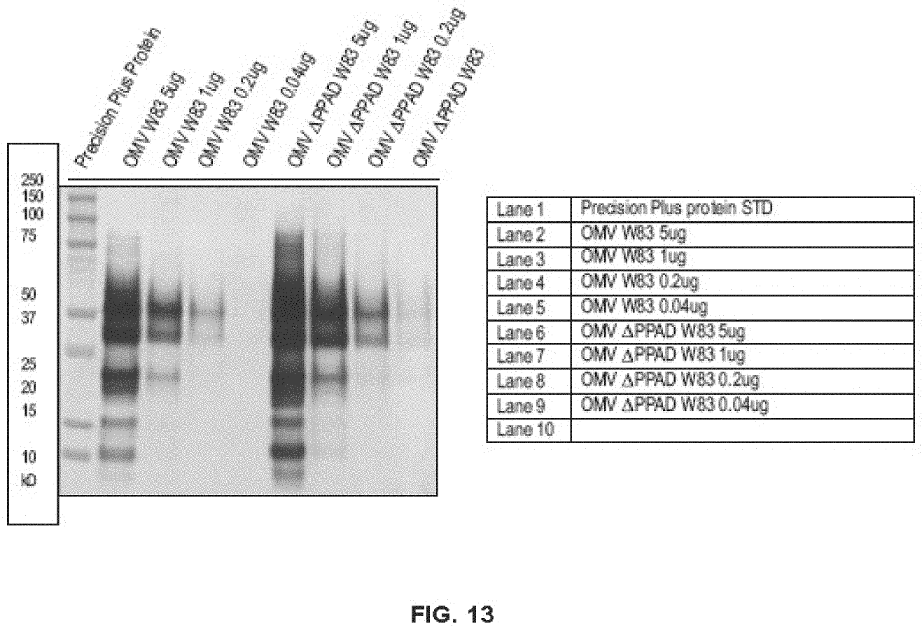

Through binding analysis, KB-001 was found to have a strong binding affinity to the outer membrane vesicles (OMV) of P.g. strain W83 (FIG. 13). FIG. 13 shows KB001 staining OMV from P. gingivalis strain 33277 and a Peptidylarginine deiminase PPAD C351A 33277 strain in a Western blot demonstrating broad binding activity against different pathogenic strains. To determine KB001 epitope, purified native gingipains were used, as well as purified His-tagged gingipains secreted by a genetically modified P. gingivalis strain. The presence of Arg in the epitope was confirmed by destruction of the epitope by incubation with RgpB which was prevented by Arg replacement with Lys. The results were then verified by expressing in E. coli K1K2K3 and R1R2R3 domains of Kgp and RgpA, respectively and proteolytic processing.

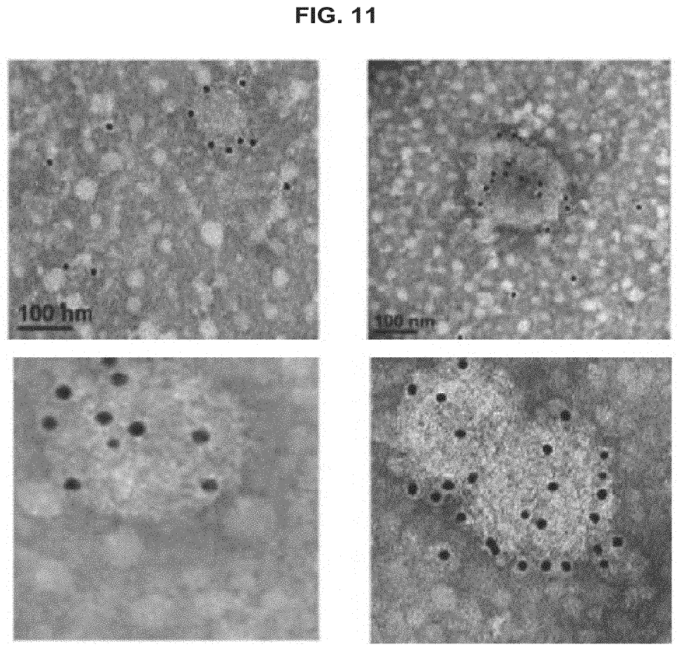

PPAD is a virulence factor unique to pathogenic Porphyromonas species, especially P. gingivalis. 100 ul Base samples (conc 500 ug/ml) and 100 μl of NuPAGE loading buffer (novex NP007) with 10% BME (Sigma M-7522) was mixed and heated at 100° C. for 10 min. 5× serial dilutions were made with cold loading buffer. Samples were electrophoresed by using 4-12% Bis-Tris SDS-PAGE (Invitrogen) at 160 v for 60 min. Subsequent analysis revealed that the major fraction of OMV particle sizes ranges from 80 to 150 mm, and KB-001 was seen to bind the inner as well as the outer surface of the OMV bleb (FIG. 11). These observations are attributed as the binding of KB-001 to a subunit toxin “XXX Epitope” complex within the secreted OMV. As disclosed herein, the “XXX Epitope” is therefore an invaluable target for vaccine therapy against both AD and P.g. infection.

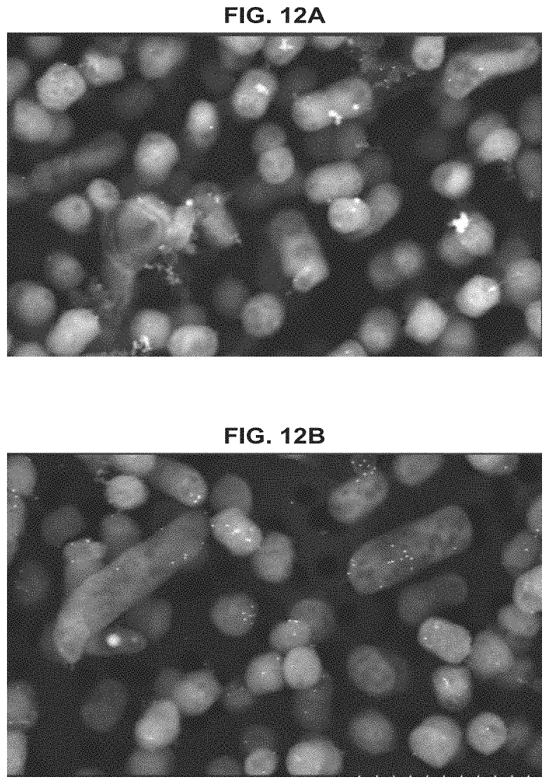

Next, the specificity of KB-001 to gingipain was screened. The binding of KB-001 to two strains: RgpA-/KgP-, and RgpB-/KgP- was monitored. Binding of the KB-001 antibody to the surface of both gingipain knock out strains in comparison to the W83 strain (FIGS. 12A and 12B) was significantly decreased (to the point of none). The W83 strain is a known gingipain-rich strain of P.g.

### Example 6

### KB-001 Activity

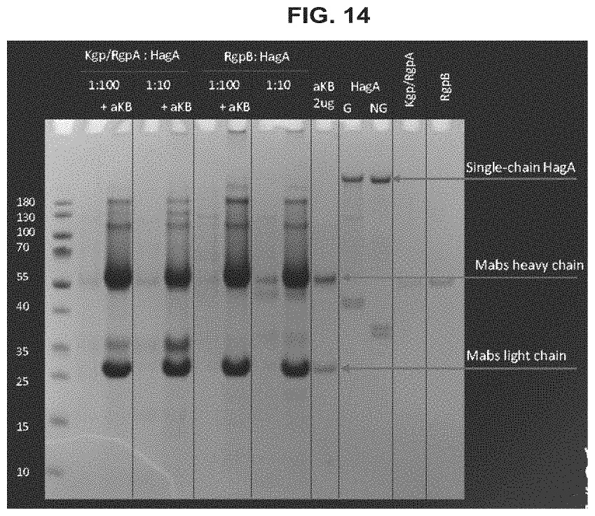

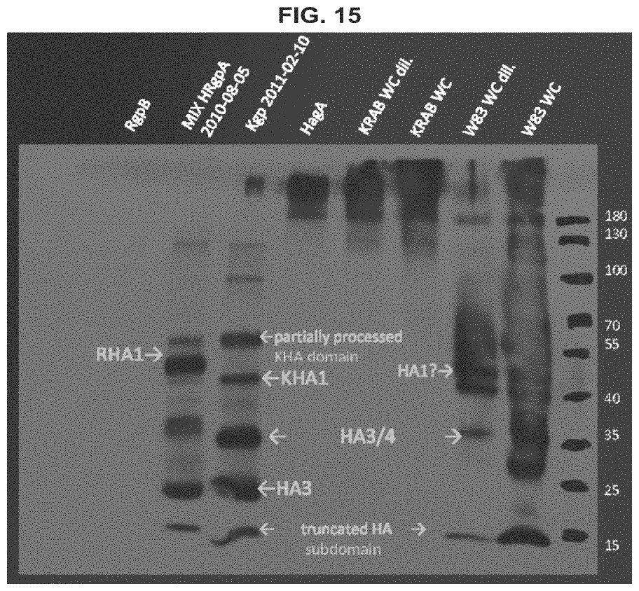

As disclosed herein, the activity of KB-001 binding in interfering with P.g. colonization, biofilm production, and virulence was assessed. It was found that KB-001 interferes with HagA processing by gingipains into the hemagglutinin/adhesion complex required for survival and adherence for colonization within the P.g. biofilm. By protein purification and Western blotting analysis, the “XXX Epitope” was found to be a single chain unique subunit toxin by gingipains. The domains of the “XXX Epitope” hold together through non-covalent interactions (FIG. 15). This is apparently the way mature “XXX Epitope” assembles on the Pg surface and in OMVs. Without boiling, this complex is stable in SDS-PAGE (FIG. 14). Upon boiling, individual HA domains can be seen on SAS-PAGE. While the present disclosure relates to generating a vaccine and/or immunogenic composition that targets P.g. and gingipain, it will be understood from this data that the “XXX Epitope” is of particular interest for targeting. It will also be understood to one skilled in the art that a superior therapy may comprise combining a vaccine and/or immunogenic composition with an effective inhibitor for P.g., gingipain, and/or the “XXX Epitope.”

### Example 7

### Immunogenic Polypeptide with YTYTVYRDGTKIK Epitope

This non-limiting example discloses an antigenic composition comprising an at least one isolated and purified peptide and/or protein, wherein the isolated and purified peptide and/or protein comprises an epitope that is bound by the antigen binding molecule KB001, and wherein the isolated and purified peptide and/or protein is capable of raising an immune response against P. gingivalis in a subject. In this example, the peptide and/or protein is not complexed with other Arg-specific and Lys-specific P. gingivalis proteinase and adhesin proteins. As disclosed herein, the composition has use in raising an immune response directed against P. gingivalis in a subject. In some alternatives, the composition has use in the prevention and/or as a therapeutic against a disease or disorder arising from the infection of P. gingivalis in a subject. In some alternatives, the subject is a human.

As disclosed herein, the composition is combined with a pharmaceutically acceptable carrier. Furthermore, the composition is formulated for oral administration to the subject. In some alternatives, the composition is formulation for subcutaneous or percutaneous administration. The peptide comprises the sequence YTYTVYRDGTKIK (SEQ ID NO: 6), which serves as a conformational epitope that binds to KB-001.

### Example 8

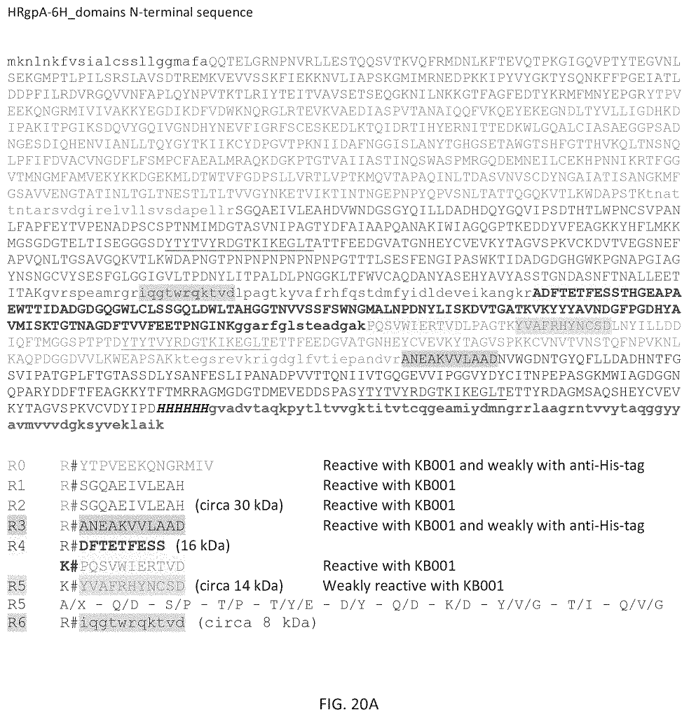

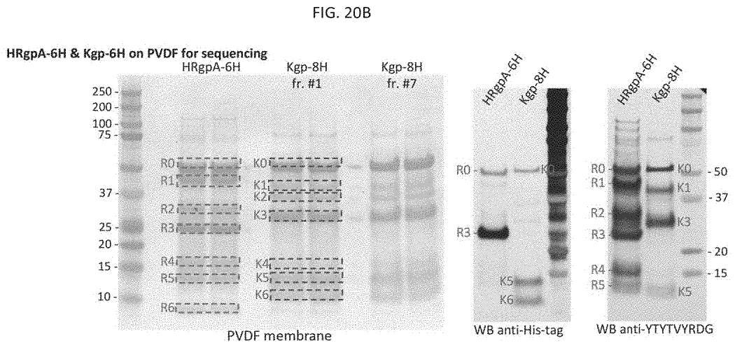

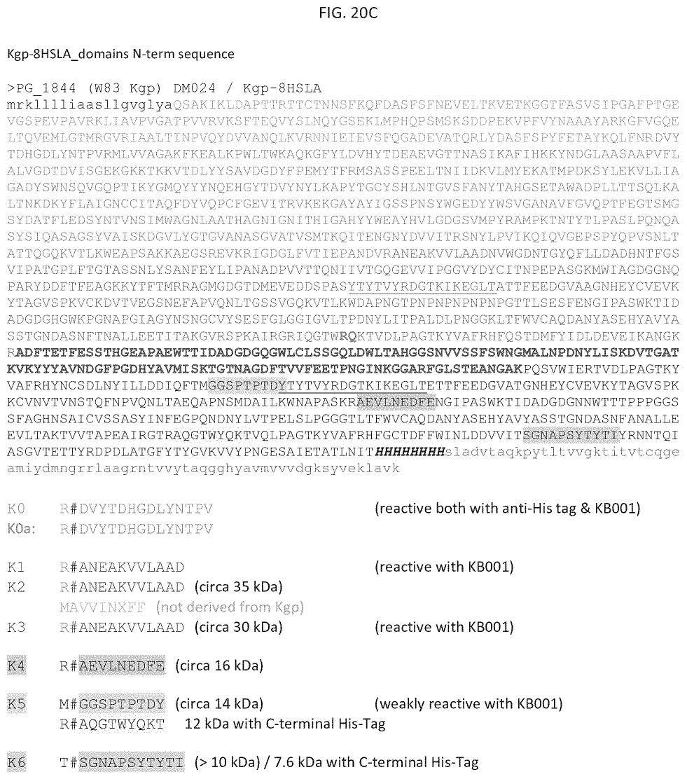

The present example outlines the process of determining the epitope for KB001, and thus a key component of the vaccine sequence. Purified native gingipains were used, as well as a series of purified domain-specific truncated His-tagged gingipains secreted by genetically modified P. gingivalis strain (FIGS. 20A and 20C for the sequence constructs and parts thereof). The presence of Arg in the epitope was confirmed by destruction of the epitope by incubation with RgpB which was prevented by Arg replacement with Lys. The results were verified by expressing in E. coli K1K2K3 and R1R2R3 domains of Kgp and RgpA, respectively and their proteolytic processing. (See FIG. 20B). The resulting sequence for which at least a part can be included in some of the vaccines provided herein is YTYTVYRDGTKIK (SEQ ID NO: 6).

### Example 9

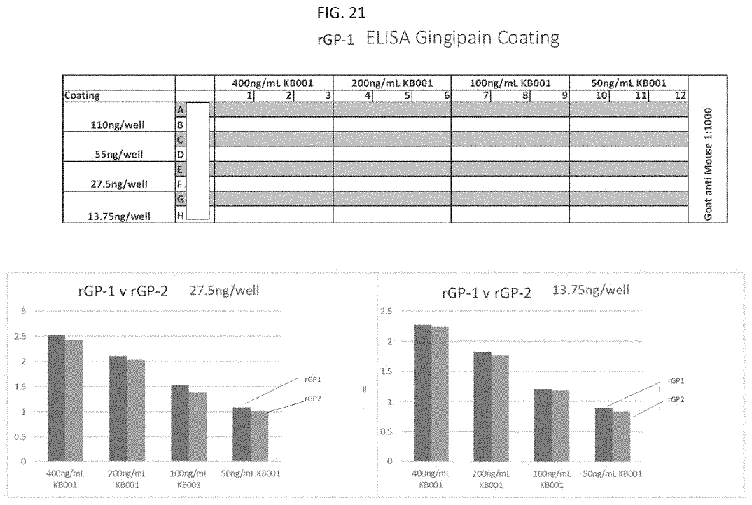

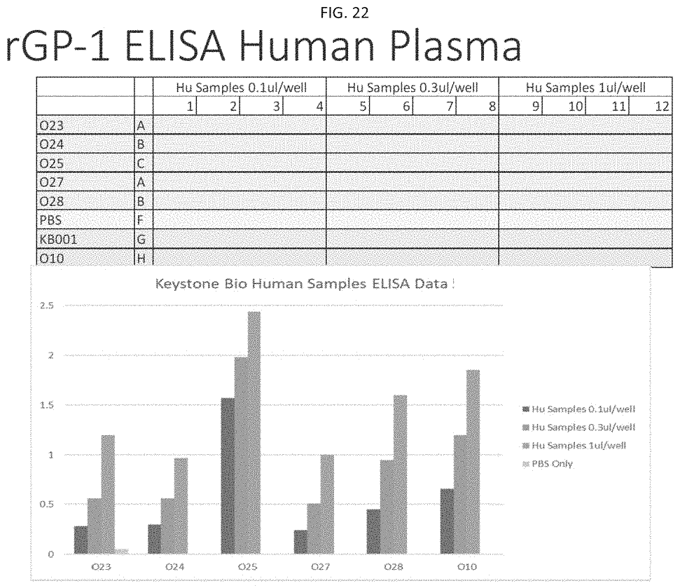

As shown in FIGS. 21 and 22. Two E. coli expressed recombinant proteins were engineered for detection of anti-gingipain antibodies. For rGP-1, the Gingipain (Gp) fragment produced with a GST fusion partner to improve solubility and purification capabilities. rGP-1 contains a single epitope recognized by KB001. A second recombinant Gp protein (rGP-2) was engineered to contain multiple KB001 epitopes. In the experiment, rGP-1 and rGP-2 proteins were assessed for use antigen in coating ELISA plates for the characterization of KB001 related antibodies. Two concentrations of each recombinant GP were used to coat wells which were probed using varying concentrations of KB001. Results from rGP-1 are shown by representative line and from rGP-2 by representative line. The data show that both recombinant proteins function well as plate-coating antigens for the detection of anti-gingipain antibodies. Also determined in this experiment is a concentration of 13.75 ng/well was sufficient for coating.

### Example 10

An ELISA approach was used for quantitating anti-gingipain antibody. The ELISA was based on quantitation of antibody binding to gingipain antigen that had been coated in the wells of 96-well plates. A recombinant GST-gingipain protein can be employed. This example demonstrates that some humans make an antibody response to this bacterial protein.

### Recombinant GST-Gingipain Protein:

Previously, the production of useful gingipain antigens for coating ELISA plates has been sporadic. Large amounts of gingipains are routinely produced from anaerobic cultures of PGW83, as demonstrated by WB and the MyBioSource ELISA analysis. However, some preparation from secreted material function well for coating plates and some did not. Because of the unpredictable nature of the purification from native sources, a GST-gingipain fusion protein was designed for expression using E. coli. The construct encodes GST (28 kDa) and a gingipain fragment (19 kDa) which contains a single KB001 epitope. The protein has a C terminal polyhistidine tract for purification and a TEV protease site between the fusion partners in the event that they need to be separated. The GST serves dual functions of increasing solubility and allowing a second method of affinity purification. The sequence of the gingipain fragment is shown below:

(SEQ ID NO: 24)
DPSCSPTNMIMDGTASVNIPAGTYDFAIAAPQANAKIWIAGQGPTKEDDY
VFEAGKKYHFLMKKMGSGDGTELTISEGGGSDYTYTVYRDGTKIKEGLTA
TTFEEDGVAAGNHEYCVEVKYTAGVSPKVCKDVTVEGSNEFAPVQNLTGS
AVGQKVTLKWDAPNGHHHHHH

The gingipain gene was synthesized and cloned into pGREX4T-1 vector in-frame with the GST gene and then transformed into BL21 DE3 pLysS cells. After performing a smallscale test, it was determined that for optimal expression of soluble protein 50 uM/mL of IPTG was necessary while shaking at 37 C for 20 hours. For production of a large amount, 10 mL of LB media containing Carbenicillin and Chloramphenicol was inoculated with bacteria and placed into a shaker at 37 C overnight. The following day this sample was added into a prewarmed flask containing 500 mL of LB media. This culture was shaken at 37 C for 2 hours. The OD was checked at regular intervals until it reached 0.45, at which time IPTG was added to a concentration of 50 uM/mL to induce expression from the T7 promoter. The flask was placed into the incubator shaker to grow for 20 hours. After this period, the cells were pelleted. The pellet was resuspended in 10 mL of 20 mM Tris Buffer pH 7.2 with 0.1% Tween and TritonX in a 50 mL tube. This sample was sonicated thoroughly then spun down and the supernatant was collected. This extraction step was repeated an additional two times for a pooled total of 30 mL. The 30 mL sample was diluted to 100 mL with 1×PBS buffer to reduce the amount of Tween and Triton and the sample was adjusted to 5 mM Imidizole to be loaded over a prepared 3 mL packed His Resin column adjusted to 5 mM Imidizole. After loading and washing, two elution fractions were taken at concentrations of 100 mM, 300 mM, 600 mM and 1M Imidazole. A WB gel with KB001 antibody at 1:10K was used to analyze 10 ul of 1/10 dilution of each sample, but the gel was blownout and needed to be re-run with less antigen. However, it was evident from the dark patch in the flow lane that all of the protein had failed to bind the first column (data not shown). A second His purification column was prepared to purify additional recombinant gingipain from the flow.

Samples from both columns were run on a western blot. The gel signal was still very strong, but individual lanes can be made out along with the control samples, and a sample of our PGW83 Lot 1. During this time, a chimeric KGP/RGP protein was run on the gel. As this gel is still very strong an additional one with even less material or reduced HRP label secondary was required. In the future, one can pool the elutions containing the target gingipain, then test these in an ELISA assay against the control samples of Lot 15 PGW83 and the rGP-2 sample. It is believed that these samples will prove strong enough and no further purification methods will be required to test the human samples.

Additional western blots with serial dilutions of the recombinant GST-Gp were able to resolve the bands in this protein. Most importantly, the recombinant GST-Gp performed very well as binding antigen in the anti-Gp ELISA (data not shown). The recombinant kGp/rGp of the rGP 2 sample also functioned well as a binding antigen. An initial analysis based on western blotting and ELISA activities compared to the rGP-2 sample antigen estimated that the concentration of the GST-Gp is approximately 0.55 mg/mL (as an estimate).

Given the results, it is believed that the production of this GST-Gingipain recombinant protein will solve the many problems surrounding reproducible purification of gingipain protein from bacterial cultures and, therefore, streamline anti-gingipain analysis by ELISA. It also demonstrates that some humans make antibodies that recognize this construct.

### Example 11

This example compares various aspects of rGP-1 and rGP-2. Pg Bacterial expressed recombinant HagA/gingipains protein: rGP-1 GST recombinant epitope: cysteines map was as follows:

(SEQ ID NO: 91)
YTYTVYRDGTKIKEGLTATTFEEDGVATGNHEYC
VEVKYTAGVSPKV
(SEQ ID NO: 92)
CKDVTVYTYTVYRDGTKIKEGLTETTFEEDGVATGNHEY
(SEQ ID NO: 93)
CVEVKYTAGVSPKK
(SEQ ID NO: 94)
CVNVTVYTYTVYRDGTKIKEGLTETTHEY
(SEQ ID NO: 95)
CVEVKYTAGVSPKVCVD

The recombinant kGp/rGp rGP-2 also functioned well as a binding antigen. Below is an amino acid seq of the Gp portion in the GST-gingipain fusion protein. Cysteines are underlined.

(SEQ ID NO: 96)
DPSCSPTNMIMDGTASVNIPAGTYDFAIAAPQANAKIWIAGQGPTK
EDDYVFEAGKKYHFLMKKMGSGDGTEL
(SEQ ID NO: 97)
TISEGGGSDYTYTVYRDGTKIKEGLTATTFEEDGVAAGNHEYCVEVKYTA
GVSPKVCKDVTVEGSNEFAPVQNLTGSAVGQKVTLKWDAPNGHHHHHH

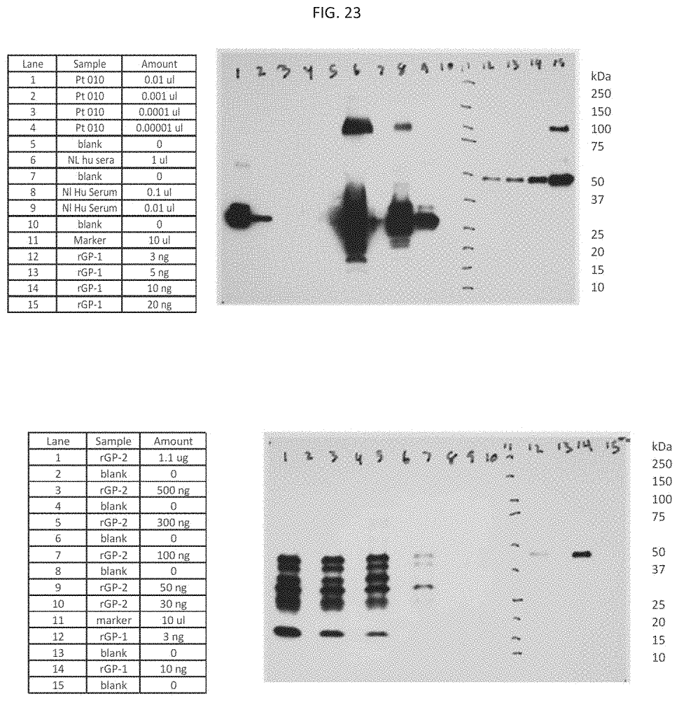

As shown in FIG. 23, the Western blot results showed that both rGP-1 (top panel) and rGP-2 (bottom panel) work for detecting gingipain. The sequence of rGP-2 is shown in FIG. 5A and 5B. Homology starts with rGP-2 using highlighted asterisks on the fifth line. rGP-1 design was to produce an antigen that would outperform native-produced gingipain from OMV for use in coating plates. rGP-1 version is the second row with rGP-2 on top. rGP-2 design appears to be useful in analyzing catalytic activity as well as having multiple eptiopes. FIG. 5B shows a direct alignment between rGP-1 and rGP-2. FIG. 54C shows a hydrophobicity plot of what could be the minimal epitope.

1-20. (canceled)

21. A method of treating a disease or condition associated with the presence of P. gingivalis in an oral tissue of a subject, comprising:
administering to the subject a peptide, wherein the peptide comprises YTYTVYRDGTKIK (SEQ ID NO: 6).

22. The method of claim 21, wherein the immunogenic composition is administered at least two times.

23-30. (canceled)

31. A method of treating or preventing a systemic disease or symptoms thereof, comprising:
identifying a subject in need of treating or preventing a systemic disease or symptoms thereof, wherein the systemic disease is one or more of type II diabetes, insulin resistance and metabolic syndrome; and
administering to the subject a therapeutically effective amount of a peptide of SEQ ID NO:6,
thereby treating or preventing the systemic disease or symptoms thereof.

32. A method of treating or preventing rheumatoid arthritis or symptoms thereof, comprising:
identifying a subject in need of treating rheumatoid arthritis or symptoms thereof; and
administering to the subject a therapeutically effective amount of a peptide of SEQ ID NO:6,
thereby treating or preventing the rheumatoid arthritis or symptoms thereof.

33-36. (canceled)

37. A method of treating or preventing cancer or symptoms thereof, comprising:
administering to a subject in need of treating or preventing cancer, or symptoms thereof, a therapeutically effective amount of at least one therapeutic agent for treating or preventing the cancer, or symptoms thereof; and
administering an effective amount of a peptide of SEQ ID NO:6, to thereby enhance the therapeutic effect of the at least one therapeutic agent.

38-39. (canceled)

40. A method of treating or preventing a gut microbiome-related disorder or symptoms thereof, comprising:
identifying a subject in need of treating a gut microbiome-related disorder or symptoms thereof; and
administering to the subject a therapeutically effective amount of a peptide of SEQ ID NO:6,
thereby treating or preventing the gut microbiome-related disorder or symptoms thereof.

41. (canceled)

42. A method of treating or preventing a cognitive disorder or symptoms thereof, comprising:
identifying a subject in need of treating a cognitive disorder or symptoms thereof; and
administering to the subject a therapeutically effective amount of a peptide of SEQ ID NO:6,
thereby treating or preventing the cognitive disorder or symptoms thereof.

43. The method of claim 42, wherein the cognitive disorder is Alzheimer's disease.

44. (canceled)

45. A method of treating or preventing an age-related or longevity-related disorder, or symptoms thereof, comprising:
identifying a subject in need of treating an age-related or longevity-related disorder; and
administering to the subject a therapeutically effective amount of a peptide of SEQ ID NO:6,
thereby treating or preventing the age-related or longevity-related disorder, or symptoms thereof.

46-51. (canceled)

52. A method of treating or preventing a large vessel stroke C-IMT or symptoms thereof, comprising:
identifying a subject in need of treating or preventing a large vessel stroke C-IMT or symptoms thereof; and
administering to the subject a therapeutically effective amount of a peptide of SEQ ID NO:6,
thereby treating or preventing the large vessel stroke C-IMT or symptoms thereof.

53-57. (canceled)

58. A method of treating or preventing a condition, disorder or disease associated with a P. gingivalis infection, or symptoms thereof, comprising:
identifying a subject in need of treating or preventing a condition, disorder or disease associated with a P. gingivalis infection, or symptoms thereof; and
administering to the subject a therapeutically effective amount of a peptide of SEQ ID NO:6,
thereby treating or preventing the condition, disorder or disease associated with a P. gingivalis infection, or symptoms thereof.

59-63. (canceled)

64. The method of any claim 31, wherein the administering comprises administering the immunogenic composition intravenously, subgingivally, intradermally, subcutaneously, intrathecally, or by nebulization.

65-66. (canceled)

67. A method of screening for a vaccine, the method comprising:
providing an antibody that binds to a Porphyromonas gingivalis protein PG0495, determining an epitope to which the antibody binds; and
providing a fragment of the Porphyromonas gingivalis protein PG0495 as a protein fragment.

68. The method of claim 67, wherein the epitope is a linear epitope.

69. The method of claim 67, wherein the epitope is a conformational epitope.

70. The method of claim 67, wherein the fragment is provided in a great enough concentration to be therapeutically effective.

71. A prokaryotic or eucaryotic cell, which comprises a recombinant polynucleotide consisting of nucleotides encoding the polypeptide of at least one of:
a polypeptide YTYTVYRDGTKIK (SEQ ID NO: 6),
a polypeptide having at least 80% identity to SEQ ID NO: 6,
an immunogenic fragment of 8, 9, 10, 12, 13, or more consecutive amino acids of SEQ ID NO: 6, and
a variant comprising a conservative substitution of at least one amino acid of SEQ ID NO: 6,
wherein the polynucleotide is operatively linked to at least one regulatory element.

72. The method of claim 21, further comprising at least one amino acid from HagA and/or gingipiain.

73. The method of claim 21, wherein the protein, epitope, polypeptide, amino acid sequence, or immunogenic fragment comprises at least one additional amino acid from HagA and/or gingipiain.

74. The method of claim 21, wherein the AP contains a set of one or more paired cysteines.

75. The method of claim 21, wherein the sequence of the peptide is at least 40, 50, 60, 70, 80, 90, 95, 96, 97, 98, 99, or 100% identical to the sequence in FIG. 4 or 5A, 5B, or 5C.

76. The method of claim 21, wherein the sequence of the peptide comprises at least YTYTVYRDGTKIK (SEQ ID NO: 6).

77. An isolated peptide or polypeptide comprising:
at least four or more amino acids from YTYTVYRDGTKIK (SEQ ID NO: 6).

78. The isolated peptide or polypeptide of claim 77, wherein the isolated peptide or polypeptide is present in a therapeutically effective amount in a vaccine.

79. The isolated peptide or polypeptide of claim 77, wherein at least 5, 6, 7, 8, 9, 10, 11, 12, or 13 of the amino acids are present in the polypeptide.

80. The isolated peptide or polypeptide of claim 77, wherein the sequence terminates at the n-terminal end, the c-terminal end, or both the n-terminal and the c-terminal end.

81. The isolated peptide or polypeptide of claim 77, wherein the sequence consists essentially of YTYTVYRDGTKIK (SEQ ID NO: 6).

82-84. (canceled)


SEQUENCE LISTING (128 sequences)

SEQ ID NO: 1 - protein, 25 aa, Artificial Sequence
  Synthetic Polypeptide
GVSPKVCKDVTVEGSNEFAPVQNLT

SEQ ID NO: 2 - protein, 14 aa, Artificial Sequence
  Synthetic Polypeptide
YCVEVKYTAGVSPK

SEQ ID NO: 3 - protein, 128 aa, Artificial Sequence
  Synthetic Polypeptide
AGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSNEFAPVQNLT

SEQ ID NO: 4 - protein, 5 aa, Artificial Sequence
  Synthetic Polypeptide
GVSPK

SEQ ID NO: 5 - protein, 1723 aa, Porphyromonas gingivalis
MRKLLLLIAASLLGVGLYAQSAKIKLDAPTTRTTCTNNSFKQFDASFSFNEVELTKVETKGGTFASVSIPGAFPTGEVGSPEVPAVRKLIAVPVGATPVVRVKSFTEQVYSLNQYGSEKLMPHQPSMSKSDDPEKVPFVYNAAAYARKGFVGQELTQVEMLGTMRGVRIAALTINPVQYDVVANQLKVRNNIEIEVSFQGADEVATQRLYDASFSPYFETAYKQLFNRDVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKTKKVTDLYYSAVDGDYFPEMYTFRMSASSPEELTNIIDKVLMYEKATMPDKSYLEKALLIAGADSYWNPKIGQQTIKYAVQYYYNQDHGYTDVYSYPKAPYTGCYSHLNTGVGFANYTAHGSETSWADPSLTATQVKALTNKDKYFLAIGNCCVTAQFDYPQPCFGEVMTRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVNMTKQITENGNYDVVITRSNYLPVIKQIQAGEPSPYQPVSNLTATTQGQKVTLKWDAPSAKKAEASREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIQEGLTATTFEEDGVAAGNHEYCVEVKYTAGVSPKVCKDVTVEGSNEFAPVQNLTGSAVGQKVTLKWDAPNGTPNPNPNPNPGTTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPEAIRGRIQGTWRQKTVDLPAGTKYVAFRHFQSTDMFYIDLDEVEIKANGKRADFTETFESSTHGEAPAEWTTIDADGDGQDWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKVCVNVTINPTQFNPVKNLKAQPDGGDVVLKWEAPSGKRGELLNEDFEGDAIPTGWTALDADGDGNNWDITLNEFTRGERHVLSPLRASNVAISYSSLLQGQEYLPLTPNNFLITPKVEGAKKITYKVGSPGLPQWSHDHYALCISKSGTAAADFEVIFEETMTYTQGGANLTREKDLPAGTKYVAFRHYNCTDVLGIMIDDVVITGEGEGPSYTYTVYRDGTKIQEGLTETTYRDAGMSAQSHEYCVEVKYAAGVSPKVCVDYIPDGVADVTAQKPYTLTVVGKTITVTCQGEAMIYDMNGRRLAAGRNTVVYTAQGGYYAVMVVVDGKSYVEKLAIK

SEQ ID NO: 6 - protein, 13 aa, Artificial Sequence
  Synthetic Polypeptide
YTYTVYRDGTKIK

SEQ ID NO: 7 - protein, 1706 aa, Artificial Sequence
  Synthetic Polypeptide
MKNLNKFVSIALCSSLLGGMAFAQQTELGRNPNVRLLESTQQSVTKVQFRMDNLKFTEVQTPKGMAQVPTYTEGVNLSEKGMPTLPILSRSLAVSDTREMKVEVVSSKFIEKKNVLIAPSKGMIMRNEDPKKIPYVYGKSYSQNKFFPGEIATLDDPFILRDVRGQVVNFAPLQYNPVTKTLRIYTEITVAVSETSEQGKNILNKKGTFAGFEDTYKRMFMNYEPGRYTPVEEKQNGRMIVIVAKKYEGDIKDFVDWKNQRGLRTEVKVAEDIASPVTANAIQQFVKQEYEKEGNDLTYVLLIGDHKDIPAKITPGIKSDQVYGQIVGNDHYNEVFIGRFSCESKEDLKTQIDRTIHYERNITTEDKWLGQALCIASAEGGPSADNGESDIQHENVIANLLTQYGYTKIIKCYDPGVTPKNIIDAFNGGISLANYTGHGSETAWGTSHFGTTHVKQLTNSNQLPFIFDVACVNGDFLFSMPCFAEALMRAQKDGKPTGTVAIIASTINQSWASPMRGQDEMNEILCEKHPNNIKRTFGGVTMNGMFAMVEKYKKDGEKMLDTWTVFGDPSLLVRTLVPTKMQVTAPAQINLTDASVNVSCDYNGAIATISANGKMFGSAVVENGTATINLTGLTNESTLTLTVVGYNKETVIKTINTNGEPNPYQPVSNLTATTQGQKVTLKWDAPSTKTNATTNTARSVDGIRELVLLSVSDAPELLRSGQAEIVLEAHDVWNDGSGYQILLDADHDQYGQVIPSDTHTLWPNCSVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSNEFAPVQNLTGSAVGQKVTLKWDAPNGTPNPNPNPNPNPNPGTTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPEAIRGRIQSTWRQKTVDLPAGTKYVAFRHFQSTDMFYIDLDEVEIKANGKRADFTETFESSTHGEATAEWTTIDADGDGQGWLCLSSGQLDWLTAHGGTNVVSSFSWNGMALNPDNYLISKDVTGATKVKYYYAVNDGFPGDHYAVMISKTGTNAGDFTVVFEETPNGINKGGARFGLSTEADGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVNSTQFNPVKNLKAQPDGGDVVLKWEAPSAKKTEGSREVKRIGDGLFVTIEPANDVRANEAKVVLAADNVWGDNTGYQFLLDADHNTFGSVIPATGPLFTGTASSDLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTETTYRDAGMSAQSHEYCVEVKYTAGVSPKVCVDYIPDGVADVTAQKPYTLTVVGKTITVTCQGEAMIYDMNGRRLAAGRNTVVYTAQGGYYAVMVVVDGKSYVKKLAIK

SEQ ID NO: 8 - protein, 51 aa, Artificial Sequence
  Synthetic Polypeptide
PASYTYTVYRDGTKIKEGLTATTFEEDGVAAGNHEYCVEVKYTAGVSPKVC

SEQ ID NO: 9 - protein, 51 aa, Artificial Sequence
  Synthetic Polypeptide
GSDYTYTVYRDGTKIKEGLTATTFEEDGVATGNHEYCVEVKYTAGVSPKVC

SEQ ID NO: 10 - protein, 51 aa, Artificial Sequence
  Synthetic Polypeptide
PTDYTYTVYRDGTKIKEGLTETTFEEDGVATGNHEYCVEVKYTAGVSPKKC

SEQ ID NO: 11 - protein, 51 aa, Artificial Sequence
  Synthetic Polypeptide
PTDYTYTVYRDGTKIKEGLTETTFEEDGVATGNHEYCVEVKYTAGVSPKEC

SEQ ID NO: 12 - protein, 51 aa, Artificial Sequence; identical to SEQ ID NO: 11
  Synthetic Polypeptide

SEQ ID NO: 13 - protein, 51 aa, Artificial Sequence; identical to SEQ ID NO: 11
  Synthetic Polypeptide

SEQ ID NO: 14 - protein, 51 aa, Artificial Sequence; identical to SEQ ID NO: 11
  Synthetic Polypeptide

SEQ ID NO: 15 - protein, 51 aa, Artificial Sequence; identical to SEQ ID NO: 11
  Synthetic Polypeptide

SEQ ID NO: 16 - protein, 51 aa, Artificial Sequence; identical to SEQ ID NO: 10
  Synthetic Polypeptide

SEQ ID NO: 17 - protein, 51 aa, Artificial Sequence
  Synthetic Polypeptide
PTDYTYTVYRDGTKIKEGLTETTFEEDGVATGNHEYCVEVKYTAGVSPKVC

SEQ ID NO: 18 - protein, 51 aa, Artificial Sequence; identical to SEQ ID NO: 11
  Synthetic Polypeptide

SEQ ID NO: 19 - protein, 51 aa, Artificial Sequence
  Synthetic Polypeptide
PASYTYTVYRDGTKIKEGLTETTYRDAGMSAQSHEYCVEVKYTAGVSPKVC

SEQ ID NO: 20 - protein, 51 aa, Artificial Sequence
  Synthetic Polypeptide
APSYTYTIYRNNTQIASGVTETTYRDPDLATGFYTYGVKVVYPNGESAIET

SEQ ID NO: 21 - protein, 736 aa, Artificial Sequence
  Synthetic Polypeptide
MKKNFSRIVSIVAFSSLLGGMAFAQPAERGRNPQVRLLSAEQSMSKVQFRMDNLQFTDVQTSKGVAQVPTFTEGVNISEKGTPILPILSRSLAVSETRAMKVEVVSSKFIEKKDVLIAPSKGVISRAENPDQIPYVYGQSYNEDKFFPGEIATLSDPFILRDVRGQVVNFAPLQYNPVTKTLRIYTEIVVAVSETAEAGQNTISLVKNSTFTGFEDIYKSVFMNYEATRYTPVEEKENGRMIVIVPKKYEEDIEDFVDWKNQRGLRTEVKVAEDIASPVTANAIQQFVKQEYEKEGNDLTYVLLVGDHKDIPAKITPGIKSDQVYGQIVGNDHYNEVFIGRFSCESKEDLKTQIDRTIHYERNITTEDKWLGQALCIASAEGGPSADNGESDIQHENVIADLLTQYGYTKIIKCYDPGVTPKNIIDAFNGGISLVNYTGHGSETAWGTSHFGTTHVKQLTNSNQLPFIFDVACVNGDFLYNVPCFAEALMRAQKDGKPTGTVAIIASTINQYWAPPMRGQDEMNEILCEKHPNNIKRTFGGVTMNGMFAMVEKYKKDGENMLDTWTVFGDPSLLVRTLVPTEMQVTAPANISASAQTFEVACDYNGAIATLSDDGDMVGTAIVKDGKAIIKLNESIADETNLTLTVVGYNKVTVIKDVKVEGTSIADVANDKPYTVAVSGKTITVESPAAGLTIFDMNGRRVATAKNRMVFEAQNGVYAVRIATEGKTYTEKVIVK

SEQ ID NO: 22 - protein, 1732 aa, Artificial Sequence
  Synthetic Polypeptide
MRKLLLLIAASLLGVGLYAQSAKIKLDAPTTRTTCTNNSFKQFDASFSFNEVELTKVETKGGTFASVSIPGAFPTGEVGSPEVPAVRKLIAVPVGATPVVRVKSFTEQVYSLNQYGSEKLMPHQPSMSKSDDPEKVPFVYNAAAYARKGFVGQELTQVEMLGTMRGVRIAALTINPVQYDVVANQLKVRNNIEIEVSFQGADEVATQRLYDASFSPYFETAYKQLFNRDVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKTKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYTAHGSETAWADPLLTTSQLKALTNKDKYFLAIGNCCITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVSMTKQITENGNYDVVITRSNYLPVIKQIQVGEPSPYQPVSNLTATTQGQKVTLKWEAPSAKKAEGSREVKRIGDGLFVTIEPANDVRANEAKVVLAADNVWGDNTGYQFLLDADHNTFGSVIPATGPLFTGTASSNLYSANFEYLVPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVDVTVNSTQFNPVQNLTAEQAPNSMDAILKWNAPASKRAEVLNEDFENGIPASWKTIDADGDGNNWTTTPPPGGSSFAGHNSAICVSSASHINFEGPQNPDNYLVTPELSLPGGGTLTFWVCAQDANYASEHYAVYASSTGNDASNFANALLEEVLTAKTVVTAPEAIRGTRAQGTWYQKTVQLPAGTKYVAFRHFGCTDFFWINLDDVVITSGNAPSYTYTIYRNNTQIASGVTETTYRDPDLATGFYTYGVKVVYPNGESAIETATLNITSLADVTAQKPYTLTVVGKTITVTCQGEAMIYDMNGRRLAAGRNTVVYTAQGGHYAVMVVVDGKSYVEKLAVK

SEQ ID NO: 23 - protein, 2105 aa, Artificial Sequence
  Synthetic Polypeptide
MARIILEAHDVWEDGTGYQMLWDADHNQYGASIPEESFWFANGTIPAGLYDPFEYKVPVNADASFSPTNFVLDGTASADIPAGTYDYVIINPNPGIIYIVGEGVSKGNDYVVEAGKTYHFTVQRQGPGDAASVVVTGEGGNEFAPVQNLQWSVSGQTVTLTWQAPASDKRTYVLNESFDTQTLPNGWTMIDADGDGHNWLSTINVYNTATHTGDGAMFSKSWTASSGAKIDLSPDNYLVTPKFTVPENGKLSYWVSSQEPWTNEHYGVFLSTTGNEAANFTIKLLEETLGSGKPAPMNLVKSEGVKAPAPYQERTIDLSAYAGQQVYLAFRHFGCTGIFRLYLDDVAVSGEGSSNDYTYTVYRDNVVIAQNLTATTFNQENVAPGQYNYCVEVKYTAGVSPKVCKDVTVEGSNEFAPVQNLTGSAVGQKVTLKWDAPNGTPNPNPGTTTLSESFENGIPASWKTIDADGDGNNWTTTPPPGGSSFAGHNSAICVSSASYINFEGPQNPDNYLVTPELSLPNGGTLTFWVCAQDANYASEHYAVYASSTGNDASNFANALLEEVLTAKTVVTAPEAIRGTRVQGTWYQKTVQLPAGTKYVAFRHFGCTDFFWINLDDVEIKANGKRADFTETFESSTHGEAPAEWTTIDADGDGQGWLCLSSGQLGWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKECVNVTVDPVQFNPVQNLTGSAVGQKVTLKWDAPNGTPNPNPGTTTLSESFENGIPASWKTIDADGDGNNWTTTPPPGGTSFAGHNSAICVSSASYINFEGPQNPDNYLVTPELSLPNGGTLTFWVCAQDANYASEHYAVYASSTGNDASNFANALLEEVLTAKTVVTAPEAIRGTRVQGTWYQKTVQLPAGTKYVAFRHFGCTDFFWINLDDVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKECVNVTVDPVQFNPVQNLTGSAVGQKVTLKWDAPNGTPNPNPGTTTLSESFENGIPASWKTIDADGDGNNWTTTPPPGGTSFAGHNSAICVSSASYINFEGPQNPDNYLVTPELSLPNGGTLTFWVCAQDANYASEHYAVYASSTGNDASNFANALLEEVLTAKTVVTAPEAIRGTRVQGTWYQKTVQLPAGTKYVAFRHFGCTDFFWINLDDVEIKANGKRADFTETFESSTHGEAPAEWTTIDADGDGQGWLCLSSGQLGWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKECVNVTINPTQFNPVQNLTAEQAPNSMDAILKWNAPASKRAEVLNEDFENGIPASWKTIDADGDGNNWTTTPPPGGSSFAGHNSAICVSSASYINFEGPQNPDNYLVTPELSLPGGGTLTFWVCAQDANYASEHYAVYASSTGNDASNFANALLEEVLTAKTVVTAPEAIRGTRVQGTWYQKTVQLPAGTKYVAFRHFGCTDFFWINLDDVVITSGNAPSYTYTIYRNNTQIASGVTETTYRDPDLATGFYTYGVKVVYPNGESAIETATLNITSLADVTAQKPYTLTVVGKTITVTCQGEAMIYDMNGRRLAAGRNTVVYTAQGGHYAVMVVVDGKSYVEKLAVK

SEQ ID NO: 24 - protein, 171 aa, Artificial Sequence
  Synthetic Polypeptide
DPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSNEFAPVQNLTGSAVGQKVTLKWDAPNGHHHHHH

SEQ ID NO: 25 - protein, 51 aa, Artificial Sequence; identical to SEQ ID NO: 8
  Synthetic Polypeptide

SEQ ID NO: 26 - protein, 51 aa, Artificial Sequence; identical to SEQ ID NO: 9
  Synthetic Polypeptide

SEQ ID NO: 27 - protein, 51 aa, Artificial Sequence; identical to SEQ ID NO: 10
  Synthetic Polypeptide

SEQ ID NO: 28 - protein, 51 aa, Artificial Sequence; identical to SEQ ID NO: 11
  Synthetic Polypeptide

SEQ ID NO: 29 - protein, 51 aa, Artificial Sequence; identical to SEQ ID NO: 17
  Synthetic Polypeptide

SEQ ID NO: 30 - protein, 51 aa, Artificial Sequence; identical to SEQ ID NO: 19
  Synthetic Polypeptide

SEQ ID NO: 31 - protein, 51 aa, Artificial Sequence; identical to SEQ ID NO: 20
  Synthetic Polypeptide

SEQ ID NO: 32 - protein, 11 aa, Artificial Sequence
  Synthetic Polypeptide
  VARIANT (6)...(6): Xaa can be any naturally occurring amino acid
DVYTDXGDLYN

SEQ ID NO: 33 - protein, 11 aa, Artificial Sequence
  Synthetic Polypeptide
  VARIANT (5)...(5): Xaa can be any naturally occurring amino acid
  VARIANT (8)...(8): Xaa can be any naturally occurring amino acid
PQSVXIEXTVD

SEQ ID NO: 34 - protein, 11 aa, Artificial Sequence
  Synthetic Polypeptide
ANEAKVVLAAD

SEQ ID NO: 35 - protein, 711 aa, Artificial Sequence
  Synthetic Polypeptide
ANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNFEAPVQNLTGSAVGQKVTLKWDAPNGTPNPNPNPNPGTTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASFNTNALLEETITAKGVRSPEAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEADGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTINPTQFNPVKNLKAQPDGGDVVLKWEAPSAKKAEGSREVKRIGDGLFVTIEPANDVR

SEQ ID NO: 36 - protein, 11 aa, Artificial Sequence
  Synthetic Polypeptide
  VARIANT (11)...(11): Xaa can be any naturally occurring amino acid
YTPVEEKQNGX

SEQ ID NO: 37 - protein, 537 aa, Artificial Sequence
  Synthetic Polypeptide
NTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSAVGQKVTLKWDAPNGTPNPNPNPNPGTTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASFNTNALLEETITAKGVRSPEAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEADGAK

SEQ ID NO: 38 - protein, 11 aa, Artificial Sequence
  Synthetic Polypeptide
  VARIANT (11)...(11): Xaa can be any naturally occurring amino acid
SGQAEIVLEAX

SEQ ID NO: 39 - protein, 417 aa, Artificial Sequence
  Synthetic Polypeptide
SGQAEIVLEAHDVWNDGSGYQILLDADHDQYGQVIPSDTHTLWPNCSVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVATGNHEYCVEVKYTAGVSPKVCKDVTVEGSNFEAPVQNLTGSAVGQKVTLKWDAPNGTPNPNPNPNPNPGTTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASFNTNALLEETITAKGVRSPEAIRGRIQGTWRQKTVDLPAGTKYVAFRHFQSTDMFYIDLDEVEIKANGKR

SEQ ID NO: 40 - protein, 418 aa, Artificial Sequence
  Synthetic Polypeptide
ANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNFEAPVQNLTGSAVGQKVTLKWDAPNGTPNPNPNPNPGTTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASFNTNALLEETITAKGVRSPEAIRGRIQGTWRQKTVDLPAGTKYVAFRHFQSTDMFYIDLDEVEIKANGKR

SEQ ID NO: 41 - protein, 373 aa, Artificial Sequence
  Synthetic Polypeptide
ANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSAVGQKVTLKWDAPNGTPNPNPNPNPGTTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASFNTNALLEETITAKGVRSPEAIRGR

SEQ ID NO: 42 - protein, 373 aa, Artificial Sequence
  Synthetic Polypeptide
ANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNFEAPVQNLTGSAVGQKVTLKWDAPNGTPNPNPNPNPGTTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASFNTNALLEETITAKGVRSPEAIRGR

SEQ ID NO: 43 - protein, 361 aa, Artificial Sequence
  Synthetic Polypeptide
PQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKECVNVTINPTQFNPVKNLKAQPDGGDVVLKWEAPSAKKTEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTETTYRDAGMSAQSHEYCVEVKYAAGVSPKVCVDYIPD

SEQ ID NO: 44 - protein, 361 aa, Artificial Sequence
  Synthetic Polypeptide
PQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTINPTQFNPVKNLKAQPDGGDVVLKWEAPSAKKAEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTETTYRDAGMSAQSHEYCVEVKYAAGVSPKVCVDYIPD

SEQ ID NO: 45 - protein, 11 aa, Artificial Sequence
  Synthetic Polypeptide
  VARIANT (7)...(7): Xaa can be any naturally occurring amino acid
  VARIANT (11)...(11): Xaa can be any naturally occurring amino acid
LPAPYQXNDIX

SEQ ID NO: 46 - protein, 203 aa, Artificial Sequence
  Synthetic Polypeptide
ANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTETTYRDAGMSAQSHEYCVEVKYAAGVSPKVCVDYIPD

SEQ ID NO: 47 - protein, 203 aa, Artificial Sequence; identical to SEQ ID NO: 46
  Synthetic Polypeptide

SEQ ID NO: 48 - protein, 373 aa, Artificial Sequence; identical to SEQ ID NO: 42
  Synthetic Polypeptide

SEQ ID NO: 49 - protein, 10 aa, Artificial Sequence
  Synthetic Polypeptide
ADFTETFESS

SEQ ID NO: 50 - protein, 135 aa, Artificial Sequence
  Synthetic Polypeptide
ADFTETFESSTHGEAPAEWTTIDADGDGQGWLCLSSGQLDWLTAHGGTNVVAFSSWNGMALNPDNYLISKDVTGATKVKYYYAVNDGFPGDHYAVMISKTGTNAGDFTVVFEETPNGINKGGARFGLSTEANGAK

SEQ ID NO: 51 - protein, 13 aa, Artificial Sequence
  Synthetic Polypeptide
IQGTWYQKTVDLP

SEQ ID NO: 52 - protein, 45 aa, Artificial Sequence
  Synthetic Polypeptide
IQGTWRQKTVDLPAGTKYVAFRHFQSTDMFYIDLDEVEIKANGKR

SEQ ID NO: 53 - protein, 1586 aa, Artificial Sequence
  Synthetic Polypeptide
MRKLLLLIAASLLGVGLYAQNAKIKLDAPTTRTTCTNNSFKQFDASFSFNEVELTKVETKGGTFASVSIPGAFPTGEVGSPEVPAVRKLIAVPVGATPVVRVKSFTEQVYSLNQYGSEKLMPHQPSMSKSDDPEKVPFAYNAAAYARKGFVGQELTQVEMLGTMRGVRIAALTINPVQYDVVANQLKVRNNIEIEVSFQGADEVATQRLYDASFSPYFETAYKQLFNRDVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKTKKVTDLYYSAVDGDYFPEMYTRFMSASSPEELTNIIDKVLMYEKATMPDKSYLEKALLIAGADSYWNPKIGQQTIKYAVQYYYNQDHGYTDVYSYPKAPYTGCYSHLNTGVGFANYTAHGSETSWADPSVTATQVKALTNKNKYFLAIGNCCVTAQFDYPQPCFGEVMTRVKEKGAYAYIGSSPNSYWGEDYYWSVGANAVFGVQPTFEGTSMGSYDATFLEDSYNTVNSIMWAGNLAATHAENIGNVTHIGAHYYWEAYHVLGDGSVMPYRAMPKTNTYTLPASLPQNQASYSIQASAGSYVAISKDGVLYGTGVANASGVATVNMTKQITENGNYDVVITRSNYLPVIKQIQAGEPSPYQPVSNLTATTQGQKVTLKWDAPSAKKAEGRSEVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSAVGQKVTLKWDAPNGTPNPNPNPNPGTTTLSESFENGIPASWKTIDADGDGHGWKPGNAPGIAGYNSNGCVYSESFGLGGIGLVTPDNYLITPALDLPNGGKLTFWVCAQDANYASEHYAVYASSTGNDASNFTNALLEETITAKGRVSPEAIRGRIQGTWRQKTVDLPAGTKYVAFRHFQSTDMFYIDLDEVEIKANGKRPQSVWIERTVDPAGTKYVAFRHYNCSDLNYILLDDIQFTMGGSPTPTDYTYTVYRDGTKIKEQLTETTFEEDGVATGNHEYCVEVKYTAGVSPKKCVNVTINPTQFNPVKNLKAQPDGGDVVLKWEAPSAKKAEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASTYTVYRDGTKIKEQLTETTYRDAGMSAGSHEYCVEVKYAAQVSPKVCVDYIPDGVADVTAQKPYTLTVVGKTITVTCQGEAMIYDMNGRRLAAGRNTVVYTAQGGYYAVMVVVDGKSYVEKLAVK

SEQ ID NO: 54 - protein, 1704 aa, Artificial Sequence
  Synthetic Polypeptide
MKNLNKFVSIALCSSLLGGMAFAQQTELGRNPNVRLLESTQQSVTKVQFRMDNLKFTEVQTPKGIGQVPTYTEGVNLSEKGMPTLPILSRSLAVSDTREMKVEVVSSKFIEKKNVLIAPSKGMIMRNEDPKKIPYVYGKSYSQNKFFPGEIATLDDPFILRDVRGQVVNFAPLQYNPVTKTLRIYTEITVAVSETSEQGKNILNKKGTFAGFEDTYKRMFMNYEPGRYTPVEEKQNGRMIVIVAKKYEGDIKDFVDWKNQRGLRTEVKVAEDIASPVTANAIQQFVKQEYEKEGNDLTYVLLVGDHKDIPAKITPGIKSDQVYGQIVGNDHYNEVFIGRFSCESKEDLKTQIDRTIHYERNITTEDKWLGQALCIASAEGGPSADNGESDIQHENVIANLLTQYGYTKIIKCYDPGVTPKNIIDAFNGGISLVNYTGHGSETAWGTSHFGTTHVKQLTNSNQLPFIFDVACVNGDFLFSMPCFAEALMRAQKDGKPTGTVAIIASTINQSWASPRMGQDEMNEILCEKHPNNIKRTFGGVTMNGMFAMVEKYKKDGEKMLDTWTVFGDPSLLVRTLVPTKMQVTAPAQINLTDASVNVSCDYNGAIATISANGKMFGSAVVENGTATINLTGLTNESTLTLTVVGYNKETVIKTINTNGEPNPYQPVSNLTATTQGQKVTLKWDAPSTKTNATTNTARSVDGIRELVLLSVSDAPELLRSGGAEIVLEAHDVWNDGSGYQILLDADHDQYQGVIPSDTHTLWPNCSVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEQLTATTFEEDGVATGNHEYCVEVKYTAQVSPKVCKDVTVEGSNEFAPVQNLTGSAVGQKVTLKWDAPNGTPNPNPNPNPNPGTTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPEAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEQLTETTFEEDGVATGNHEYCVEVKYTAQVSPKECVNVTINPTGFNPVKNLKAGPDGGDVVLKWEAPSAKKTEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEQLTETTYRDAGMSAGSHEYCVEVKYAAQVSPKVCVDYIPDGVADVTAQKPYTLTVVGKTITVTCQGEAMIYDMNGRRLAAGRNTVVYTAQGGYYAVMVVVDGKSYVEKLAVK

SEQ ID NO: 55 - protein, 441 aa, Artificial Sequence
  Synthetic Polypeptide
EVQLKQSGPGLVAPSQSLSITCTVSGFSLSIYSVHWVRQPPGKGLEWLGMIWGGGSSDYNSALKSRLSISKDNSKSQVFLKMNSLQTDDTAMYYCARNGNFYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 56 - protein, 239 aa, Artificial Sequence
  Synthetic Polypeptide
MDFQVQIFSFLLISASVIMSRGQIVLTQSPAIMSASLGERVTMTCTASSSVSSSFLHWYQQKPGSSPQLWIYSTSNLASGVPARFSGSGSGTSYSLTISSMEAEDAATYYCHQYHHSPYIYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 57 - protein, 78 aa, Artificial Sequence
  Synthetic Polypeptide
YTYTVYRDGTKIKEGLTATTFEEDGVAAGNHEYCVEVKYTAGVSPKVCKDVTVEGSNEFAPVQNLTGSAVGQKVTLKW

SEQ ID NO: 58 - protein, 77 aa, Artificial Sequence
  Synthetic Polypeptide
YTYTVYRDGTKIKEGLTETTFEEDGVATGNHEYCVEVKYTAGVSPKKCVNVTINPTQFNPVKNLKAQPDGGDVVLKW

SEQ ID NO: 59 - protein, 54 aa, Artificial Sequence
  Synthetic Polypeptide
YTYTVYRDGTKIKEGLTETTYRDAGMSAQSHEYCVEVKYAAGVSPKVCVDYIPD

SEQ ID NO: 60 - protein, 78 aa, Artificial Sequence
  Synthetic Polypeptide
YTYTVYRDGTKIKEGLTATTFEEDGVATGNHEYCVEVKYTAGVSPKVCKDVTVEGSNEFAPVQNLTGSAVGQKVTLKW

SEQ ID NO: 61 - protein, 77 aa, Artificial Sequence
  Synthetic Polypeptide
YTYTVYRDGTKIKEGLTETTFEEDGVATGNHEYCVEVKYTAGVSPKECVNVTINPTQFNPVKNLKAQPDGGDVVLKW

SEQ ID NO: 62 - protein, 78 aa, Artificial Sequence
  Synthetic Polypeptide
YTYTVYRDGTKIQEGLTATTFEEDGVAAGNHEYCVEVKYTAGVSPKVCKDVTVEGSNEFAPVQNLTGSAVGQKVTLKW

SEQ ID NO: 63 - protein, 77 aa, Artificial Sequence
  Synthetic Polypeptide
YTYTVYRDGTKIKEGLTETTFEEDGVATGNHEYCVEVKYTAGVSPKVCVNVTINPTQFNPVKNLKAQPDGGDVVLKW

SEQ ID NO: 64 - protein, 55 aa, Artificial Sequence
  Synthetic Polypeptide
YTYTVYRDGTKIQEGLTETTYRDAGMSAQSHEYCVEVKYAAGVSPKVCVDYIPDG

SEQ ID NO: 65 - protein, 53 aa, Artificial Sequence
  Synthetic Polypeptide
YTYTIYRNNTQIASGVTETTYRDPDLATGFYTYGVKVVYPNGESAIETATLNI

SEQ ID NO: 66 - protein, 55 aa, Artificial Sequence
  Synthetic Polypeptide
YTYTVYRDGTKIKEGLTETTYRDAGMSAQSHEYCVEVKYAAGVSPKVCVDYIPDG

SEQ ID NO: 67 - protein, 77 aa, Artificial Sequence
  Synthetic Polypeptide
YTYTVYRDGTKIKEGLTETTFEEDGVATGNHEYCVEVKYTAGVSPKKCVNVTVNSTQFNPVKNLKAQPDGGDVVLKW

SEQ ID NO: 68 - protein, 54 aa, Artificial Sequence
  Synthetic Polypeptide
YTYTVYRDGTKIKEGLTETTYRDAGMSAQSHEYCVEVKYTAGVSPKVCVDYIPD

SEQ ID NO: 69 - protein, 54 aa, Artificial Sequence; identical to SEQ ID NO: 59
  Synthetic Polypeptide

SEQ ID NO: 70 - protein, 54 aa, Artificial Sequence
  Synthetic Polypeptide
YTYTVYRDNVVIAQNLTATTFNQENVAPGQYNYCVEVKYTAGVSPKVCKDVTVE

SEQ ID NO: 71 - protein, 54 aa, Artificial Sequence
  Synthetic Polypeptide
YTYTVYRDGTKIKEGLTETTFEEDGVATGNHEYCVEVKYTAGVSPKECVNVTVD

SEQ ID NO: 72 - protein, 54 aa, Artificial Sequence
  Synthetic Polypeptide
YTYTVYRDGTKIKEGLTETTFEEDGVATGNHEYCVEVKYTAGVSPKECVNVTIN

SEQ ID NO: 73 - protein, 54 aa, Artificial Sequence
  Synthetic Polypeptide
YTYTIYRNNTQIASGVTETTYRDPDLATGFYTYGVKVVYPNGESAIETATLNIT

SEQ ID NO: 74 - protein, 2618 aa, Artificial Sequence
  Synthetic Polypeptide
MRKLNSLFSLAVLLSLLCWGQTAAAQGGPKTAPSVTHQAVQKGIRTSKVKDLRDPIPAGMARIILEAHDVWEDGTGYQMLWDADHNQYGASIPEESFWFANGTIPAGLYDPFEYKVPVNADASFSPTNFVLDGTASADIPAGTYDYVIINPNPGIIYIVGEGVSKGNDYVVEAGKTYHFTVQRQGPGDAASWVTGEGGNEFAPVQNLQWSVSGQTVTLTWQAPASDKRTYVLNESFDTQTLPNGWTMIDADGDGHNWLSTINVYNTATHTGDGAFMSKSWTASGGAKIDLSPDNYLVTPKVTVPENGKLSYWVSSQVPWTNEHYGVFLSTTGNEAANFTIKLLEETLGSDKPAPMNLVKSEGVKLPAPYQERTIDLSAYAGQQVYLAFRHFNSTGIFRLYLDDVAVSGEGSSNDYTYTVYRDNWIAQNLAATTFNQENVAPGQYNYCVEVKYTAGVSPKVCKDVTVEGSNEFAPVQNLTGSAVGQKVTLKWDAPNGTPNPNPGTTTLSEFSENGIPASWKTIDADGDGNNWTTTPPPGGTSFAGHNSAICASSASYINFEGPQNPDNYLVTPELSLPNGGTLTFWVCAQDANYASEHYAVYASSTGNDASNFANALLEEVLTAKTVVTAPEAIRGTRVQGTWYQKTVQLPAGTKYVFARHFGCTDFFWINLDDVEIKANGKRADFTETFESSTHGEAPAEWTTIDADGDGQGWLCLSSGQLGWLTAHGGTNVVASFSWNGMALNPDNYLISKDVTGATKVKYYYAVNDGFPGDHYAVMISKTGTNAGDFTVVFEETPNGINKGGARFGLSTEADGAKPQSVWIERTVDLPAGTKYVAFRHYNCSDLNYILLDDIQFTMGGSPTPTDYTYTVYRDGTKIKEGLTETFTEEDGVATGNHEYCVEVKYTAGVSPKECVNVTVDPVQFNPVQNLTGSAVGQKVTLKWDAPNGTPNPNPNPNPGTTTLSESFENGIPASWKTIDADGDGNNWTTTPPPGGTSFAGHNSAICASSASYINFEGPQNPDNYLVTPELSLPNGGTLTFWVCAQDANYASEHYAVYASSTGNDASNFANALLEEVLTAKTVVTAPEAIRGTRVQGTWYQKTVQLPAGTKYVFARHFGCTDFFWINLDDVEIKANGKRADFTETFESSTHGEAPAEWTTIDADGDGQGWLCLSSGQLGWLTAHGGTNVVAFSSWNGMALNPDNYLISKDVTGATKVKYYYAVNDGFPGDHYAVMISKTGTNAGDFTWEETPNGINKGGARFGLSTEADGAKPQSVWIERTVDLPAGTKYVAFRHYNCSDLNYILLDDIQFTMGGSPTPTDYTYTVYRDGTKIKEGLTETFTEEDGVATGNHEYCVEVKYTAGVSPKECVNVTVDPVFQNPVQNLTGSAVGQKVTLKWDAPNGTPNPNPNPNPGTTTLSESFENGIPASWKTIDADGDGNNWTTTPPPGGTSFAGHNSAICVSSASYINFEGPQNPDNYLVTPELSLPGGGTLTFWVCAQDANYASEHYAVYASSTGNDASNFANALLEEVLTAKTVVTAPEAIRGTRVQGTWYQKTVQLPAGTKYVAFRHFGCTDFFWINLDDVEIKANGKRADFTETFESSTHGEAPAEWTTIDADGDGQGWLCLSSGQLGWLTAHGGTNVVASFSWNGMALNPDNYLISKDVTGATKVKYYYAVNDGFPGDHYAVMISKTGTNAGDFTWFEETPNGINKGGARFGLSTEADGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKECVNVTVDPVFQNPVQNLTGSAVGQKVTLKWDAPNGTPNPNPNPNPGTTTLSESFENGIPASWKTIDADGDGNNWTTTPPPGGTSFAGHNSAICVSSASYINFEGPQNPDNYLVTPELSLPGGGTLTFWVCAQDANYASEHYAVYASSTGNDASNFANALLEEVLTAKTWTAPEAIRGTRVQGTWYQKTVQLPAGTKYVAFRHFGCTDFFWINLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTWFEETPNGINKGGARFGLSTEADGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKVCVNVITNPTQFNPVQNLTAEQAPNSMDAILKWNAPASKRAEVLNEDFENGIPSSWKTIDADGDGNNWTTTPPPGGSSFAGHNSAICVSSASYINFEGPQNPDNYLVTPELSLPGGGTLTFWVCAQDANYASEHYAVYASSTGNDASNFANALLEEVLTAKTVVTAPEAIRGTRVQGTWYQKTVQLPAGTKYVAFRHFGCTDFFWINLDDVVITSGNAPSYTYTIYRNNTQIASGVTETTYRDPDLATGFYTYGVKWPNGESAIETATLNITSLADVTAQKPYTLTWGKTITVTCQGEAMIYDMNGRRLAAGRNTVVYTAQGGHYAVMVVVDGKSYVEKLAVK

SEQ ID NO: 75 - protein, 54 aa, Artificial Sequence
  Synthetic Polypeptide
YTYTVYRDNVVIAQNLAATTFNQENVAPGQYNYCVEVKYTAGVSPKVCKDVTVE

SEQ ID NO: 76 - protein, 54 aa, Artificial Sequence
  Synthetic Polypeptide
YTYTVYRDGTKIKEGLTETTFEEDGVATGNHEYCVEVKYTAGVSPKVCVNVTIN

SEQ ID NO: 77 - protein, 55 aa, Artificial Sequence
  Synthetic Polypeptide
YTYTVYRDGTKIQEGLTATTFEEDGVAAGNHEYCVEVKYTAGVSPKVCKDVTVEG

SEQ ID NO: 78 - protein, 55 aa, Artificial Sequence
  Synthetic Polypeptide
YTYTVYRDGTKIKEGLTETTFEEDGVATGNHEYCVEVKYTAGVSPKVCVNVTINP

SEQ ID NO: 79 - protein, 55 aa, Artificial Sequence
  Synthetic Polypeptide
YTYTVYRDGTKIKEGLTATTFEEDGVAAGNHEYCVEVKYTAGVSPKVCKDVTVEG

SEQ ID NO: 80 - protein, 55 aa, Artificial Sequence
  Synthetic Polypeptide
YTYTVYRDGTKIKEGLTETTFEEDGVATGNHEYCVEVKYTAGVSPKKCVNVTINP

SEQ ID NO: 81 - protein, 55 aa, Artificial Sequence
  Synthetic Polypeptide
YTYTVYRDGTKIKEGLTATTFEEDGVATGNHEYCVEVKYTAGVSPKVCKDVTVEG

SEQ ID NO: 82 - protein, 55 aa, Artificial Sequence
  Synthetic Polypeptide
YTYTVYRDGTKIKEGLTETTFEEDGVATGNHEYCVEVKYTAGVSPKECVNVTINP

SEQ ID NO: 83 - protein, 55 aa, Artificial Sequence
  Synthetic Polypeptide
YTYTVYRDGTKIKEGLTETTFEEDGVATGNHEYCVEVKYTAGVSPKKCVNVTVNS

SEQ ID NO: 84 - protein, 53 aa, Artificial Sequence
  Synthetic Polypeptide
YTYTVYRDGTKIKEGLTATTFEEDGVATGNHEYCVEVKYTAGVSPKVCKDVTV

SEQ ID NO: 85 - DNA, 585 bp, Unknown
  Synthetic Polynucleotide
cgagctcggtacctcgcgaatgcatctagatgcggatccgcagagaatctgtactttcaaggagcagatccgagctgtagtccaacgaatatgattatggatggcaccgcatctgtcaacattccagccggaacctacgattttgctattgccgcaccacaagcaaatgcaaaaatttggatcgcaggacaaggaccaaccaaagaagatgattatgtgtttgaagcggggaagaaatatcactttctgatgaaaaaaatgggcagtggggatggaaccgaattgacgattagcgaagggggaggctcagattatacatacaccgtataccgggatggtactaaaattaaagaaggtttaacagcaacaacgtttgaagaggatggcgtagcagcgggtaatcacgaatattgtgtagaagtaaagtatactgccggagtgtcacctaaagtgtgtaaagatgtaacagttgaaggtagtaacgaatttgcgccggtacaaaatttaacgggtagtgcagtgggccagaaagtaactttgaaatgggatgcgccaaatggtcaccaccatcatcatcattaatag

SEQ ID NO: 86 - DNA, 720 bp, Artificial Sequence
  Synthetic Polynucleotide
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccccggaattcccgggtcgactcgagcggccgcatcgtgactga

SEQ ID NO: 87 - protein, 413 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDRARYLANASRCGSAENLYFQGADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSNEFAPVQNLTGSAVGQKVTLKWDAPNGHHHHHH

SEQ ID NO: 88 - protein, 1285 aa, Artificial Sequence
  Synthetic Polypeptide
YTPVEEKQNGRMIVIVAKKYEGDIKDFVDWKNQRGLRTEVKVAEDIASPVTANAIQQFVKQEYEKEGNDLTYVLLIGDHKDIPAKITPGIKSDQVYGQIVGNDHYNEVFIGRFSCESKEDLKTQIDRTIHYERNITTEDKWLGQALCIASAEGGPSADNGESDIQHENVIANLLTQYGYTKIIKCYDPGVTPKNIIDAFNGGISLANYTGHGSETAWGTSHFGTTHVKQLTNSNQLPFIFDVACVNGDFLFSMPCFAEALMRAQKDGKPTGTVAIIASTINQSWASPMRGQDEMNEILCEKHPNNIKRTFGGVTMNGMFAMVEKYKKDGEKMLDTWTVFGDPSLLVRTLVPTKMQVTAPAQINLTDASVNVSCDYNGAIATISANGKMFGSAVVENGTATINLTGLTNESTLTLTVVGYNKETVIKTINTNGEPNPYQPVSNLTATTQGQKVTLKWDAPSTKSGQAEIVLEAHDVWNDGSGYQILLDADHDQYGQVIPSDTHTLWPNCSVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVATGNHEYCVEVKYTAGVSPKVCKDVTVEGSNEFAPVQNLTGSAVGQKVTLKWDAPNGTPNPNPNPNPNPNPGTTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKADFTETFESSTHGEAPAEWTTIDADGDGQGWLCLSSGQLDWLTAHGGTNVVSSFSWNGMALNPDNYLISKDVTGATKVKYYYAVNDGFPGDHYAVMISKTGTNAGDFTVVFEETPNGINKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVNSTQFNPVKNLKAQPDGGDVVLKWEAPSAKANEAKVVLAADNVWGDNTGYQFLLDADHNTFGSVIPATGPLFTGTASSDLYSANFESLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTETTYRDAGMSAQSHEYCVEVKYTAGVSPKVCVDYIPHHHHHH

SEQ ID NO: 89 - protein, 226 aa, Artificial Sequence
  Synthetic Polypeptide
LWPNCSVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVATGNHEYCVEVKYTAGVSPKVCKDVTVEGSNEFAPVQNLTGSAVGQKVTLKWDAPNGTPNPNPNPNPNPNPGTTTSESFENGIPASWKTIDADGD

SEQ ID NO: 90 - protein, 171 aa, Artificial Sequence; identical to SEQ ID NO: 24
  Synthetic Polypeptide

SEQ ID NO: 91 - protein, 47 aa, Artificial Sequence
  Synthetic Polypeptide
YTYTVYRDGTKIKEGLTATTFEEDGVATGNHEYCVEVKYTAGVSPKV

SEQ ID NO: 92 - protein, 39 aa, Artificial Sequence
  Synthetic Polypeptide
CKDVTVYTYTVYRDGTKIKEGLTETTFEEDGVATGNHEY

SEQ ID NO: 93 - protein, 14 aa, Artificial Sequence
  Synthetic Polypeptide
CVEVKYTAGVSPKK

SEQ ID NO: 94 - protein, 29 aa, Artificial Sequence
  Synthetic Polypeptide
CVNVTVYTYTVYRDGTKIKEGLTETTHEY

SEQ ID NO: 95 - protein, 17 aa, Artificial Sequence
  Synthetic Polypeptide
CVEVKYTAGVSPKVCVD

SEQ ID NO: 96 - protein, 72 aa, Artificial Sequence
  Synthetic Polypeptide
DPSCSPTNMIMDGTASVNIPAGTYDFAIAAPQANAKIWIAGQGPTKEDDYVFEAGKKYHFLMKKMGSGDGTE

SEQ ID NO: 97 - protein, 98 aa, Artificial Sequence
  Synthetic Polypeptide
TISEGGGSDYTYTVYRDGTKIKEGLTATTFEEDGVAAGNHEYCVEVKYTAGVSPKVCKDVTVEGSNEFAPVQNLTGSAVGQKVTLKWDAPNGHHHHHH

SEQ ID NO: 98 - protein, 51 aa, Artificial Sequence; identical to SEQ ID NO: 8
  Synthetic Polypeptide

SEQ ID NO: 99 - protein, 51 aa, Artificial Sequence; identical to SEQ ID NO: 9
  Synthetic Polypeptide

SEQ ID NO: 100 - protein, 51 aa, Artificial Sequence; identical to SEQ ID NO: 10
  Synthetic Polypeptide

SEQ ID NO: 101 - protein, 51 aa, Artiicial Sequence; identical to SEQ ID NO: 11
  Synthetic Polypeptide

SEQ ID NO: 102 - protein, 51 aa, Artificial Sequence; identical to SEQ ID NO: 17
  Synthetic Polypeptide

SEQ ID NO: 103 - protein, 51 aa, Artificial Sequence; identical to SEQ ID NO: 19
  Synthetic Polypeptide

SEQ ID NO: 104 - protein, 51 aa, Artificial Sequence; identical to SEQ ID NO: 20
  Synthetic Polypeptide

SEQ ID NO: 105 - protein, 458 aa, Artificial Sequence
  Synthetic Polypeptide
RADFTETFESSTHGEAPAEWTTIDADGDGQGWLCLSSGQLGWLTAHGGTNVVASFSWNGMALNPDNYLISKDVTGATKVKYYYAVNDGFPFDHYAVMISMTGTNAGDFTVVFEETPNGINKGGARFGLSTEADGAKPQSVWIERTVFLPAGTKYVAFRHYNCSDLNYILLDDIWFTMGGSPTPTDYTYTVYRDGTKIKEGLTETTFEEDGVATGNHEYCVEVKYTAGVSPKECVNVTVDPVQFNPWQNLTGSAVGQKVTLKWDAPNGTPNPNPNPNPGTTTLSESFENGIPASWKTIDADGDGNNWTTTPPPGGTSFAGHNSAICASSASYINFEGPQNPDNYLVTPELSLPNGGTLTFWVCAQDANYASEHYAVYASSTGNDASNFANALLEEVLTAKTVVTAPEAIRGTRVQGTWYQKTVQLPAGTKYVAFRHFGCTDFFWINLDDVEIKANGKRA

SEQ ID NO: 106 - protein, 458 aa, Artificial Sequence; identical to SEQ ID NO: 105
  Synthetic Polypeptide

SEQ ID NO: 107 - protein, 456 aa, Artificial Sequence
  Synthetic Polypeptide
RADFTETFESSTHGEAPAEWTTIDADGDGQGWLCLSSGQLGWLTAHGGTNVVASFSWNGMALNPDNYLISKDVTGATKVKYYYAVNDGFPGDHYAVMISKTGTNAGDFTVVFEETPNGINKGGARFGLSTEADGAKPQSVWIERTVDLPAGTKYVAFRHYNCSDLNYILLDDIQFTMGGSPTPTDYTYTVYRDGTKKIKEGLTETTFEEDGVATGNHEYCVEVKYTAGVSPKECVNVTVDPVQFNPWQNLTGSAVGQKKVTLKWDAPNGTPNPNPNPNPGTTTLSASFENGIPASWKTIDADGDGNNQTTTPPPGGTSFAGHNSAICSSASYINFEGPQNPDNYLCTPELSPGGGTLTFWVCAQDANYASEHYAVYASSTGNDASNFANALEEVLTAKTVVTAPEAIRGTRVQGTWYQKTVQLPAGTKYVAFRHFGCTDFFWINLDEVEIKANKRA

SEQ ID NO: 108 - protein, 508 aa, Artificial Sequence
  Synthetic Polypeptide
RADFTETFESSTHGEAPAEWTTIDADGDGDQGWLCLSSGQLDWLTAHGGTNVVASFSWNGMALNPADNYLISKDVTGATKVKYYYAVNDGPFGDHYAVMISKTGTNAGDFTVVFEETPNGINKGGARFGLSTEADGAKPQSVWIERTVDLPAGTKYVAFRHYNCSDLNYILLDDIQFTMGGSPTPTDYTYTVYRDGTKIKEGLTETTFEEDGVATGNHEYGVEVKYTAGVSPKVCVNVTINPTQFNPWQNLTAEQAPNSMDAILKWNAPASKRAEVLNEDFENGIPSSWKTIDADGDGNNWTTTPPPGGSSFAGHNSAICVSSASYINFEGPQNPDNYLVTPELSLPGGGTLTFWVCAQDANYASEHYAVYASSTGNDASNFANALLEEVLTAKTVVTAPEAIRGTRVQGTWYQKTVQLPAGTKYVAFRHFGCTDFFWINLDDVVITSGNAPSYTYTIYRNNTQIASGVTETTYRDPDLATGFYTYGVKVVYPNGESAIETATLNITS

SEQ ID NO: 109 - protein, 452 aa, Artificial Sequence
  Synthetic Polypeptide
ADFTETFESSTHGEAPAEWTTIDADGDGQGWLCLSSGQLGWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKECVNVTVDPVQFNPVQNLTGSAVGQKVTLKWDAPNGTPNPNPGTTTLSESFENGIPASWKTIDADGDGNNWTTTPPPGGTSFAGHNSAICVSSASYINFEGPQNPDNYLVTPELSLPNGGTLTFWVCAQDANYASEHYAVYASSTGNDASNFANALLEEVLTAKTVVTAPEAIRGTRVQGTWYQKTVQLPAGTKYVAFRHFGCTDFFWINLDDVEIKANGKR

SEQ ID NO: 110 - protein, 452 aa, Artificial Sequence
  Synthetic Polypeptide
ADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKECVNVTVDPVQFNPVQNLTGSAVGQKVTLKWDAPNGTPNPNPGTTTLSESFENGIPASWKTIDADGDGNNWTTTPPPGGTSFAGHNSAICVSSASYINFEGPQNPDNYLVTPELSLPNGGTLTFWVCAQDANYASEHYAVYASSTGNDASNFANALLEEVLTAKTVVTAPEAIRGTRVQGTWYQKTVQLPAGTKYVAFRHFGCTDFFWINLDDVEIKANGKR

SEQ ID NO: 111 - protein, 504 aa, Artificial Sequence
  Synthetic Polypeptide
ADFTETFESSTHGEAPAEWTTIDADGDGQGWLCLSSGQLGWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKECVNVTINPTQFNPVQNLTAEQAPNSMDAILKWNAPASKRAEVLNEDFENGIPASWKTIDADGDGNNWTTTPPPGGSSFAGHNSAICVSSASYINFEGPQNPDNYLVTPELSLPGGGTLTFWVCAQDANYASEHYAVYASSTGNDASNFANALLEEVLTAKTVVTAPEAIRGTRVQGTWYQKTVQLPAGTKYVAFRHFGCTDFFWINLDDVVITSGNAPSYTYTIYRNNTQIASGVTETTYRDPDLATGFYTYGVKVVYPNGESAIETATLNIT

SEQ ID NO: 112 - protein, 421 aa, Artificial Sequence
  Synthetic Polypeptide
RANEAKVVLAAADNVWGDNTGYQFLLDADHNTFGSVIPATGPLFTGTASSNLYSANFEYLVPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVECKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGKR

SEQ ID NO: 113 - protein, 505 aa, Artificial Sequence
  Synthetic Polypeptide
ADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVDVTVNSTQFNPVQNLTAEQAPNSMDAILKWNAPASKRAEVLNEDFENGIPASWKTIDADGDGNNWTTTPPPGGSSFAGHNSAICVSSASHINFEGPQNPDNYLVTPELSLPGGGTLTFWVCAQDANYASEHYAVYASSTGNDASNFANALLEEVLTAKTVVTAPEAIRGTRAQGTWYQKTVQLPAGTKYVAFRHFGCTDFFWINLDDVVITSGNAPSYTYTIYRNNTQIASGVTETTYRDPDLATGFYTYGVKVVYPNGESAIETATLNITS

SEQ ID NO: 114 - protein, 421 aa, Artificial Sequence
  Synthetic Polypeptide
RSGQAEIVLEAHDVWNDGSGYQILLDADHDQYGQVIPSDTHTLWPNCSVPANLFAPFEYTVPENADPSCSPTNMIMDGTASVNIPAGTYDFAIAAPQANAKIWIAGQGPTKEDDYWFEAGKKYHFLMKKMGSGDGTELTISEGGGSDYTYTVYRDGTKIKEGLTATTFEEDGVATGNHEYCVEVKYTAGVSPKVCKDVTVEGSNEFAPVQNLTGSAVGQKVTLKWDAPNGTPNPNPNPNPNPNPGTTTLSESFENGIPASWKTIDADGDGHGWKPGNAPGIAGYNSNGCVYSESFGLLGGIGVLTPDNYLITPALDLPNGGKLTFWVCAQDANYASEHYAVYASSTGNDASNFTNALLEETITAKGVRSPEAMRGRIQGTWRQKTVDLPAGTKYVAFRHFQSTDMFYIDLDEVEIKANGKR

SEQ ID NO: 115 - protein, 496 aa, Artificial Sequence
  Synthetic Polypeptide
ADFTETFESSTHGEAPAEWTTIDADGDGQGWLCLSSGQLDWLTAHGGTNVVSSFSWNGMALNPDNYLISKDVTGATKVKYYYAVNDGFPGDHYAVMISKTGTNAGDFTVVFEETPNGINKGGARFGLSTEADGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVNSTQFNPVKNLKAQPDGGDVVLKWEAPSAKKTEGSREVKRIGDGLFVTIEPANDVRANEAKVVLAADNVWGDNTGYQFLLDADHNTFGSVIPATGPLFTGTASSDLYSANFESLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTETTYRDAGMSAQAHEYCVEWKYTAGVSPKVCVDYIPD

SEQ ID NO: 116 - protein, 925 aa, Artificial Sequence
  Synthetic Polypeptide
RANEAKVVLAADNVWGDNTGYQFLLDADHNTFGSVIPATGPLFTGTASSNLYSANFEYLVPANADPVVTTQNIIVGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIGGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVDVTVNSTQFNPVQNLTAEQAPNSMDAILKWNAPASKRAEVLNEDFENGIPASWKTIDADGDGNNWTTTPPPGGSSFAGHNSAICVSSASHINFEGPQNPDNYLVTPELSLPGGGTLTFWVCAQDANYASEHYAVYASSTGNDASNFANALLEEVLTAKTVVTAPEAIRGTRAQGTWYQKTVQLPAGTKYWAFRHFGCTDFFWINLDDVVITSGNAPSYTYTIYRNNTQIASGVTETTYRDPDLATGFYTYGVKVVYPNGESAIETATLNITS

SEQ ID NO: 117 - protein, 916 aa, Artificial Sequence
  Synthetic Polypeptide
RSGQAEIVLLEAHDVQNDGSGYQILLDADHDQYGQVIPSDTHTLWPNCSVPANLFAPFEYTVPENADPSCSPTNMIMDGTASVNIPAGTYDFAIAAPQANAKIWIAGQGPTKEDDYVFEAGKKYHFIMKKMGSDGTELTISEGGSDYTYTVYRDGTKIKEGLTATTFEEDGVATGNHEYCVEVKYTAGVSPKVCKDVTVEGNSNEFAPVQNLTGSAVGQKVTLKWDAPNGTPNPNPNPNPNPNPGTTTLSESFENGIPASWKTIDADGDGHGWKPGNAPGIAGYNSNGCVYSESPGLGGIGVLTPDNYLITPALDLPNGGKLTFWVCAQDANYASEHYAVYASSTGNDASNFTNALLEETITAKGVRSPEAMRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVSSFSWNGMALNPDNYLISKDVTGATKVKYYYAVNDGFPGDHYAVMISKTGTNAGDFTVVFEETPNGINKGGARFGLSTEADGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVNSTQFNPVKNLKAQPDGGDVVLKWEAPSAKKTEGSREVKRIGDGLFVTIEPANDVRANEAKVVLAADNVWGDNTGYQFLLDADHNTFGSVIPATGPLFTGTASSDLYSANFESLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTETTYRDAGMSAQSHEYCVEVKYTAGVSPKVCVDYIPD

SEQ ID NO: 118 - protein, 502 aa, Artificial Sequence
  Synthetic Polypeptide
ADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEADGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKVCVNVTINPTQFNPVQNLTAEQAPNSMDAILKWNAPASKRAEVLNEDFENGIPSSWKTIDADGDGNNWTTTPPPGGSSFAGHNSAICVSSASYINFEGPQNPDNYLVTPELSLPGGGTLTFWVCAQDANYASEHYAVYASSTGNDASNFANALLEEVLTAKTVVTAPEAIRGTRVQGTWYQKTVQLPAGTKYVAFRHFGCTDFFWINLDDWITSGNAPSYTYTIYRNNTQIASGVTETTYRDPDLATGFYTYGVKWYPNGESAIETATLNIT

SEQ ID NO: 119 - protein, 456 aa, Artificial Sequence
  Synthetic Polypeptide
ADFTETFESSTHGEAPAEWTTIDADGDGQGWLCLSSGQLGWLTAHGGTNVVASFSWNGMALNPDNYLISKDVTGATKVKYYYAVNDGFPGDHYAVMISKTGTNAGDFTVVFEETPNGINKGGARFGLSTEADGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKECVNVTVDPVQFNPVQNLTGSAVGQKVTLKWDAPNGTPNPNPNPNPGTTTLSESFENGIPASWKTIDADGDGNNWTTTPPPGGTSFAGHNSAICASSASYINFEGPQNPDNYLVTPELSLPNGGTLTFWVCAQDANYASEHYAVYASSTGNDASNFANALLEEVLTAKTVVTAPEAIRGTRVQGTWYQKTVQLPAGTKYVAFRHFGCTDFFWINLDDVEIKANGKR

SEQ ID NO: 120 - protein, 456 aa, Artificial Sequence
  Synthetic Polypeptide
ADFTETFESSTHGEAPAEWTTIDADGDGQGWLCLSSGQLGWLTAHGGTNVVASFSWNGMALNPDNYLISKDVTGATKVKYYYAVNDGFPGDHYAVMISKTGTNAGDFTVVFEETPNGINKGGARFGLSTEADGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKECVNVTVDPVQFNPVQNLTGSAVGQKVTLKWDAPNGTPNPNPNPNPGTTTLSESFENGIPASWKTIDADGDGNNWTTTPPPGGTSFAGHNSAICVSSASYINFEGPQNPDNYLVTPELSLPGGGTLTFWVCAQDANYASEHYAVYASSTGNDASNFANALLEEVLTAKTVVTAPEAIRGTRVQGTWYQKTVQLPAGTKYVAFRHFGCTDFFWINLDDVEIKANGKR

SEQ ID NO: 121 - protein, 456 aa, Artificial Sequence
  Synthetic Polypeptide
ADFTETFESSTHGEAPAEWTTIDADGDGQGWLCLSSGQLGWLTAHGGTNVVASFSWNGMALNPDNYLISKDVTGATKVKYYYAVNDGFPGDHYAVMISKTGTNAGDFTVVFEETPNGINKGGARFGLSTEADGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKECVNVTVDPVQFNPVQNLTGSAVGQKVTLKWDAPNGTPNPNPNPNPGTTTLSESFENGIPASWKTIDADGDGNNWTTTPPPGGTSFAGHNSAICVSSASYINFEGPQNPDNYLVTPELSLPGGGTLTFWVCAQDANYASEHYAVYASSTGNDASNFANALLEEVLTAKTVVTAPEAIRGTRVQGTWYQKTVQLPAGTKYVAFRHFGCTDFFWINLDEVEIKANGKR

SEQ ID NO: 122 - protein, 503 aa, Artificial Sequence
  Synthetic Polypeptide
ADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTWFEETPNGINKGGARFGLSTEADGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKVCVNVTINPTQFNPVQNLTAEQAPNSMDAILKWNAPASKRAEVLNEDFENGIPSSWKTIDADGDGNNWTTTPPPGGSSFAGHNSAICVSSASYINFEGPQNPDNYLVTPELSLPGGGTLTFWVCAQDANYASEHYAVYASSTGNDASNFANALLEEVLTAKTVVTAPEAIRGTRVQGTWYQKTVQLPAGTKYVAFRHFGCTDFFWINLDDVVITSGNAPSYTYTIYRNNTQIASGVTETTYRDPDLATGFYTYGVKVVYPNGESAIETATLNIT

SEQ ID NO: 123 - protein, 450 aa, Artificial Sequence
  Synthetic Polypeptide
ANEAKWLAADNVWGDNTGYQFLLDADHNTFGSVIPATGPLFTGTASSNLYSANFEYLVPANADPVVTTQNIIVTGQGEWIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTDVLYTYTVYRDGTKIKEGLTETTFEEDGVATGNHEYPAGTKYVAFRHFQSTDMFYIDLDEVEIKANGKR

SEQ ID NO: 124 - protein, 419 aa, Artificial Sequence
  Synthetic Polypeptide
SGQAEIVLEAHDVWNDGSGYQILLDADHDQYGQVIPSDTHTLWPNCSVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVATGNHEYCVEVKYTAGVSPKVCKDVTVEGSNEFAPVQNLTGSAVGQKVTLKWDAPNGTPNPNPNPNPNPNPGTTTLSESFENGIPASWKTIDADGDGHGWKPGNAPGIAGYNSNGCVYSESFGLGGIGVLTPDNYLITPALDLPNGGKLTFWVCAQADNYASEHYAVYASSTGNDASNFTNALLEETITAKGVRSPEAMRGRIQGTWRQKTVDLPAGTKYVAFRHFQSTDMFYIDLDEVEIKANGKR

SEQ ID NO: 125 - protein, 439 aa, Artificial Sequence
  Synthetic Polypeptide
ADFTETFESSTHGEAPAEWTTIDADGDGQGWLCLSSGQLGWLTAHGGTNWASFSWNGMALNPDNYLISKDVTGATKVKYYYAVNDGFPGDHYAVMISKTGTNAGDFTVVFEETPNGINKPQSVWIERTVDLPAGTKYVAFRHYNCSDLNYILLDDIQFTMGGSPTPTDYTYTVYRDGTKIKELGTETTFEEDGVATGNHEYCVEVKYTAGVSPKECVNVTVDPVQFNPVQNLTGSAVGQKVTLKWDAPNGTPNPNPNPNPGTTTLSESFENGIPASWKTIDADGDGNNWTTTPPPGGTSFAGHNSAICASSASYINFEGPQNPDNYLVTPELSLPNGGTLTFWVCAQDANYASEHYAVYASSTGNDASNFANALLEEVLTAKTWTAPEAIRGTRVQGTWYQKTVQLPAGTKYVAFRHFGCTDFFWINLDDVEIKANGKR

SEQ ID NO: 126 - protein, 439 aa, Artficial Sequence
  Synthetic Polypeptide
ADFTETFESSTHGEAPAEWTTIDADGDGQGWLCLSSGQLGWLTAHGGTNWASFSWNGMALNPDNYLISKDVTGATKVKYYYAVNDGFPGDHYAVMISKTGTNAGDFTVVFEETPNGINKPQSVWIERTVDLPAGTKYVAFRHYNCSDLNYILLDDIQFTMGGSPTPTDYTYTVYRDGTKIKELGTETTFEEDGVATGNHEYCVEVKYTAGVSPKECVNVTVDPVQFNPVQNLTGSAVGQKVTLKWDAPNGTPNPNPNPNPGTTTLSESFENGIPASWKTIDADGDGNNWTTTPPPGGTSFAGHNSAICVSSASYINFEGPQNPDNYLVTPELSLPGGGTLTFWVCAQDANYASEHYAVYASSTGNDASNFANALLEEVLTAKTWTAPEAIRGTRVQGTWYQKTVQLPAGTKYVAFRHFGCTDFFWINLDDVEIKANGKR

SEQ ID NO: 127 - protein, 439 aa, Artificial Sequence
  Synthetic Polypeptide
ADFTETFESSTHGEAPAEWTTIDADGDGQGWLCLSSGQLGWLTAHGGTNWASFSWNGMALNPDNYLISKDVTGATKVKYYYAVNDGFPGDHYAVMISKTGTNAGDFTVVFEETPNGINKPQSVWIERTVDLPAGTKYVAFRHYNCSDLNYILLDDIQFTMGGSPTPTDYTYTVYRDGTKIKELGTETTFEEDGVATGNHEYCVEVKYTAGVSPKECVNVTVDPVQFNPVQNLTGSAVGQKVTLKWDAPNGTPNPNPNPNPGTTTLSESFENGIPASWKTIDADGDGNNWTTTPPPGGTSFAGHNSAICVSSASYINFEGPQNPDNYLVTPELSLPGGGTLTFWVCAQDANYASEHYAVYASSTGNDASNFANALLEEVLTAKTWTAPEAIRGTRVQGTWYQKTVQLPAGTKYVAFRHFGCTDFFWINLDEVEIKANGKR

SEQ ID NO: 128 - protein, 447 aa, Artificial Sequence
  Synthetic Polypeptide
ADFTETFESSTHGEAPAEWTTIDADGDGQGWLCLSSGQLDWLTAHGGTNWASFSWNGMALNPDNYLISKDVTGATKVKYYYAVNDGFPGDHYAVMISKTGTNAGDFTVVFEETPNGINKPQSVWIERTVDLPAGTKYVAFRHYNCSDLNYILLDDIQFTMGGSPTPTDYTYTVYRDGTKIKELGTETTFEEDGVATGNHEYCVEVKYTAGVSPKVCVNVTINPTQFNPVQNLTAEQAPNSMDAILKWNAPASKFAGHNSAICVSSASYINFEGPQNPDNYLVTPELSLPGGGTLTFWVCAQDANYASEHYAVYASSTGNDASNFANALLEEVLTAKTWTAPEAIRGTRVQGTWYQKTVQLPAGTKYVAFRHFGCTDFFWINLDDWITSGNAPSYTYTIYRNNTQIASGVTETTYRDPDLATGFYTYGVKVVYPNGESAIETATLNIT